(19) United States
(12) Patent Application Publication
(10) Pub. No.: US 2024/0166740 A1
(43) Pub. Date: May 23, 2024

(54) ANTI-LAG3 ANTIBODIES AND USES THEREOF

(71) Applicant: Regeneron Pharmaceuticals, Inc., Tarrytown, NY (US)

(72) Inventors: Erica ULLMAN, Yorktown Heights, NY (US); Aynur HERMANN, Jersey City, NJ (US); Ella IOFFE, Bronx, NY (US); Elena BUROVA, Mount Kisco, NY (US); Gavin THURSTON, Briarcliff Manor, NY (US)

(21) Appl. No.: 18/317,241

(22) Filed: May 15, 2023

Related U.S. Application Data
(63) Continuation of application No. 16/433,263, filed on Jun. 6, 2019, now Pat. No. 11,692,032.
Application No. 16/433,263 is a continuation of application No. 15/289,032, filed on Oct. 7, 2016, now Pat. No. 10,358,495.
(60) Provisional application No. 62/239,524, filed on Oct. 9, 2015.
(60) Provisional application No. 62/257,791, filed on Nov. 20, 2015.
(60) Provisional application No. 62/315,119, filed on Mar. 30, 2016.
(60) Provisional application No. 62/359,921, filed on Jul. 8, 2016.
(60) Provisional application No. 62/365,006, filed on Jul. 21, 2016.

(51) Int. Cl. C07K 16/28; A61K 39/395; A61K 45/06

(52) U.S. Cl. CPC C07K 16/2803; A61K 39/3955; A61K 45/06; C07K 16/2818; A61K 2039/505

(57) ABSTRACT

The present invention provides antibodies that bind to the T cell co-inhibitor lymphocyte activation gene 3 (LAG3) protein, and methods of use. In various embodiments of the invention, the antibodies are fully human antibodies that specifically bind to LAG3. In some embodiments, the antibodies of the invention are useful for inhibiting or neutralizing LAG3 activity, thus providing a means of treating a disease or disorder such as cancer or viral infection.

### CROSS-REFERENCE TO RELATED APPLICATIONS

This application is a continuation of U.S. patent application Ser. No. 16/433,263, filed Jun. 6, 2019, which is a continuation of U.S. patent application Ser. No. 15/289,032, filed Oct. 7, 2016, now U.S. Pat. No. 10,358,495, which claims the benefit under 35 U.S.C. § 119(e) of U.S. Provisional Application Nos. 62/239,524, filed Oct. 9, 2015; 62/257,791, filed Nov. 20, 2015; 62/315,119, filed Mar. 30, 2016; 62/359,921, filed Jul. 8, 2016; and 62/365,006, filed Jul. 21, 2016, the disclosures of each herein incorporated by reference in their entireties.

### SEQUENCE LISTING

An official copy of the sequence listing is submitted concurrently with the specification electronically via Patent Center as an XML formatted sequence. The contents of the electronic sequence listing (10176US03 Sequence Listing ST26.xml; Size 708,608 bytes; and Date of Creation: May 15, 2023) is herein incorporated by reference in its entirety.

### FIELD OF THE INVENTION

The present invention is related to antibodies and antigen-binding fragments of antibodies that specifically bind to the immunomodulatory receptor lymphocyte activation gene-3 (LAG3), and therapeutic and diagnostic methods of using those antibodies.

### BACKGROUND OF THE INVENTION

T cell co-stimulatory and co-inhibitory molecules (collectively named co-signaling molecules) play a crucial role in regulating T cell activation, subset differentiation, effector function and survival (Chen et al 2013, Nature Rev. Immunol. 13: 227-242). Following recognition of cognate peptide-MHC complexes on antigen-presenting cells by the T cell receptor, co-signaling receptors co-localize with T cell receptors at the immune synapse, where they synergize with TCR signaling to promote or inhibit T cell activation and function (Flies et al 2011, Yale J. Biol. Med. 84: 409-421). The ultimate immune response is regulated by a balance between co-stimulatory and co-inhibitory signals (“immune checkpoints”) (Pardoll 2012, Nature Reviews Cancer 12: 252-264). Lymphocyte activation gene-3 (LAG3) functions as one such ‘immune checkpoint’ in mediating peripheral T cell tolerance.

LAG3 (also called CD223) is a 503 amino acid transmembrane protein receptor expressed on activated CD4 and CD8 T cells, γδ T cells, natural killer T cells, B-cells, natural killer cells, plasmacytoid dendritic cells and regulatory T cells. LAG3 is a member of the immunoglobulin (Ig) superfamily. The primary function of LAG3 is to attenuate the immune response. LAG3 binding to MHC class II molecules results in delivery of a negative signal to LAG3-expressing cells and down-regulates antigen-dependent CD4 and CD8 T cell responses. LAG3 negatively regulates the ability of T cells to proliferate, produce cytokines and lyse target cells, termed as ‘exhaustion’ of T cells. LAG3 is also reported to play a role in enhancing T regulatory (Treg) cell function (Pardoll 2012, Nature Reviews Cancer 12: 252-264).

Since LAG3 plays an important role in tumor immunity and infectious immunity, it is an ideal target for immunotherapy. Blocking LAG3 with antagonists, including monoclonal antibodies, has been studied in treatments of cancer and chronic viral infections (Turnis et al 2015, Eur. J. Immunol. 45: 1892-1905).

Monoclonal antibodies to LAG3 are known in the art and have been described, for example, in US Patent/Publication Nos. 5976877, 6143273, 6197524, 8551481, 20110070238, 20110150892, 20130095114, 20140093511, 20140127226, 20140286935, and in WO95/30750, WO97/03695, WO98/58059, WO2004/078928, WO2008/132601, WO2010/019570, WO2014/008218, EP0510079B1, EP0758383B1, EP0843557B1, EP0977856B1, EP1897548B2, EP2142210A1, and EP2320940B1.

When developing an immunotherapy for treating human beings, there is a need for antibodies exhibiting properties such as low immunogenicity, suitable binding kinetics parameters, cross-reactivity to the monkey target, suitable in vitro activity and/or suitable in vivo activity.

### BRIEF SUMMARY OF THE INVENTION

The present invention provides antibodies and antigen-binding fragments thereof that bind LAG3. The antibodies of the present invention are useful, inter alia, for targeting immune cells expressing LAG3, and for modulating LAG3 activity. In certain embodiments, the antibodies of the invention are useful for inhibiting or neutralizing LAG3 activity and/or for stimulating T cell activation, e.g., under circumstances where T cell-mediated killing is beneficial or desirable. In certain embodiments, the antibodies are useful for inhibiting regulatory T cell function and/or for reversing the anergic state of exhausted T cells. The anti-LAG3 antibodies of the invention, or antigen-binding portions thereof, may be included as part of a multi-specific antigen-binding molecule, for example, to modulate the immune response and/or to target the antibodies to a specific cell type, such as a tumor cell, or a virally infected cell. The antibodies are useful in treating a disease or disorder such as cancer and viral infection.

The antibodies of the invention can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., 2000, J. Immunol. 164:1925-1933). In certain embodiments, the antibodies may be bispecific.

In a first aspect, the present invention provides isolated recombinant monoclonal antibodies or antigen-binding fragments thereof that bind specifically to LAG3. In certain embodiments, the antibodies are fully human.

Exemplary anti-LAG3 antibodies of the present invention are listed in Tables 1-3 herein. Table 1 sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-LAG3 antibodies. Table 2 sets forth the nucleic acid sequence identifiers of the HCVRs, LCVRs, HCDR1, HCDR2 HCDR3, LCDR1, LCDR2 and LCDR3 of the exemplary anti-LAG3 antibodies. Table 3 sets forth the amino acid sequence identifiers of heavy chain and light chain sequences of exemplary anti-LAG3 antibodies.

The present invention provides antibodies, or antigen-binding fragments thereof, comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present invention provides antibodies, or antigen-binding fragments thereof, comprising an HCVR/LCVR amino acid sequence pair contained within any of the exemplary anti-LAG3 antibodies listed in Table 1. In certain embodiments, the HCVR/LCVR amino acid sequence pair is selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/522, 458/522, 466/522, 474/522, 482/522, 490/522, 498/530, 506/530, 514/530, 538/546, and 554/562. In certain embodiments, the HCVR/LCVR amino acid sequence pair is selected from one of SEQ ID NOs: 386/394 (e.g., H4sH15479P), 418/426 (e.g., H4sH15482P) or 538/546 (e.g., H4sH14813N). In certain embodiments, the present invention provides anti-LAG3 antibodies or antigen-binding fragments thereof comprising a HCVR and a LCVR, said HCVR comprising an amino acid sequence listed in Table 1 having no more than five amino acid substitutions, and said LCVR comprising an amino acid sequence listed in Table 1 having no more than five amino acid substitutions. For example, the present invention provides anti-LAG3 antibodies or antigen-binding fragments thereof comprising a HCVR and a LCVR, said HCVR comprising an amino acid sequence of SEQ ID NO: 418 having no more than five amino acid substitutions, and said LCVR comprising an amino acid sequence of SEQ ID NO: 426 having no more than five amino acid substitutions. In another exemplary embodiment, the present invention provides anti-LAG3 antibodies or antigen-binding fragments thereof comprising a HCVR and a LCVR, said HCVR comprising an amino acid sequence of SEQ ID NO: 418 having at least one amino acid substitution, and said LCVR comprising an amino acid sequence of SEQ ID NO: 426 having one amino acid substitution.

The present invention also provides antibodies, or antigen-binding fragments thereof, comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present invention provides antibodies, or antigen-binding fragments thereof, comprising an HCDR3/LCDR3 amino acid sequence pair contained within any of the exemplary anti-LAG3 antibodies listed in Table 1. In certain embodiments, the HCDR3/LCDR3 amino acid sequence pair is selected from the group consisting of SEQ ID NOs: 392/400 (e.g., H4sH15479P), 424/432 (e.g., H4sH15482P) and 544/552 (e.g., H4sH14813N).

The present invention also provides antibodies, or antigen-binding fragments thereof, comprising a HCVR and a LCVR, said HCVR comprising HCDR1 comprising an amino acid sequence differing from an amino acid sequence listed in Table 1 by 1 amino acid, HCDR2 comprising an amino acid sequence differing from an amino acid sequence listed in Table 1 by 1 amino acid, and HCDR3 comprising an amino acid sequence differing from an amino acid sequence listed in Table 1 by 1 amino acid. In certain embodiments, the present invention provides antibodies, or antigen-binding fragments thereof, comprising a HCVR and a LCVR, said LCVR comprising LCDR1 comprising an amino acid sequence differing from an amino acid sequence listed in Table 1 by 1 amino acid, LCDR2 comprising an amino acid sequence differing from an amino acid sequence listed in Table 1 by 1 amino acid, and LCDR3 comprising an amino acid sequence differing from an amino acid sequence listed in Table 1 by 1 amino acid. For example, the present invention provides anti-LAG3 antibodies, or antigen-binding fragments thereof, comprising a HCVR and a LCVR, said HCVR comprising HCDR1 comprising an amino acid sequence of SEQ ID NO: 420 or an amino acid sequence differing from SEQ ID NO: 420 by 1 amino acid, HCDR2 comprising an amino acid sequence of SEQ ID NO: 422 or an amino acid sequence differing from SEQ ID NO: 422 by 1 amino acid, and HCDR3 comprising an amino acid sequence of SEQ ID NO: 424 or an amino acid sequence differing from SEQ ID NO: 424 by 1 amino acid. In another exemplary embodiment, the present invention provides antibodies, or antigen-binding fragments thereof, comprising a HCVR and a LCVR, said LCVR comprising LCDR1 comprising an amino acid sequence of SEQ ID NO: 428 or an amino acid sequence differing from SEQ ID NO: 428 by 1 amino acid, LCDR2 comprising an amino acid sequence of SEQ ID NO: 430 or an amino acid sequence differing from SEQ ID NO: 430 by 1 amino acid, and LCDR3 comprising an amino acid sequence of SEQ ID NO: 432 or an amino acid sequence differing from SEQ ID NO: 432 by 1 amino acid.

The present invention provides antibodies, or antigen-binding fragments thereof, comprising a heavy chain comprising an amino acid sequence selected from any of the HC amino acid sequences listed in Table 3, or a substantially similar sequence thereof having at least 90%, at least 95%, at least 98% or at least 99% sequence identity thereto.

The present invention also provides antibodies, or antigen-binding fragments thereof, comprising a light chain comprising an amino acid sequence selected from any of the LC amino acid sequences listed in Table 3, or a substantially similar sequence thereof having at least 90%, at least 95%, at least 98% or at least 99% sequence identity thereto.

The present invention also provides antibodies, or antigen-binding fragments thereof, comprising a HC and a LC amino acid sequence pair (HC/LC) comprising any of the HC amino acid sequences listed in Table 3 paired with any of the LC amino acid sequences listed in Table 3.

According to certain embodiments, the present invention provides antibodies, or antigen-binding fragments thereof, comprising an HC/LC amino acid sequence pair contained within any of the exemplary anti-LAG3 antibodies listed in Table 3. In certain embodiments, the HC/LC amino acid sequence pair is selected from the group consisting of SEQ ID NOs: 577/578, 579/578, and 580/581.

The present invention also provides antibodies, or antigen-binding fragments thereof, comprising a set of six CDRs (i.e., HCDR1-HCDR2-HCDR3-LCDR1-LCDR2-LCDR3) contained within any of the exemplary anti-LAG3 antibodies listed in Table 1. In certain embodiments, the HCDR1-HCDR2-HCDR3-LCDR1-LCDR2-LCDR3 amino acid sequence set is selected from the group consisting of SEQ ID NOs: 388-390-392-396-398-400 (e.g., H4sH15479P), 420-422-424-428-430-432 (e.g., H4sH15482P) and 540-542-544-548-550-552 (e.g., H4sH14813N).

In a related embodiment, the present invention provides antibodies, or antigen-binding fragments thereof, comprising a set of six CDRs (i.e., HCDR1-HCDR2-HCDR3-LCDR1-LCDR2-LCDR3) contained within an HCVR/LCVR amino acid sequence pair as defined by any of the exemplary anti-LAG3 antibodies listed in Table 1. For example, the present invention includes antibodies, or antigen-binding fragments thereof, comprising the HCDR1-HCDR2-HCDR3-LCDR1-LCDR2-LCDR3 amino acid sequences set contained within an HCVR/LCVR amino acid sequence pair selected from the group consisting of SEQ ID NOs: 386/394 (e.g., H4sH15479P), 418/426 (e.g., H4sH15482P) and 538/546 (e.g., H4sH14813N). Methods and techniques for identifying CDRs within HCVR and LCVR amino acid sequences are well known in the art and can be used to identify CDRs within the specified HCVR and/or LCVR amino acid sequences disclosed herein.

Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et aL., J. MoL. Biol. 273:927-948 (1997); and Martin et aL., Proc. Natl. Acad. Sci. USA 86:9268-9272 (1989). Public databases are also available for identifying CDR sequences within an antibody.

The present invention includes anti-LAG3 antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

The present invention includes anti-LAG3 antibodies comprising a Fc domain, wherein the Fc domain comprises IgG1 or IgG4 isotype as described elsewhere herein. In certain embodiments, the Fc domain comprises an amino acid sequence selected from the group consisting of SEQ ID NOs: 569, 570, 571, 572 and 573.

The present invention also provides for antibodies and antigen-binding fragments thereof that compete for specific binding to LAG3 with an antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

The present invention also provides isolated antibodies and antigen-binding fragments thereof that block LAG3 binding to MHC class II. In some embodiments, the antibody or antigen-binding fragment thereof that blocks LAG3 binding may bind to the same epitope on LAG3 as MHC class II or may bind to a different epitope on LAG3 as MHC class II.

The present invention also provides antibodies and antigen-binding fragments thereof that bind specifically to LAG3 from human or other species. In certain embodiments, the antibodies may bind to human LAG3 and/or to monkey LAG3.

The present invention also provides antibodies and antigen-binding fragments thereof that cross-compete for binding to LAG3 with a reference antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

The present invention also provides antibodies and antigen-binding fragments thereof that bind to the same epitope as a reference antibody or antigen-binding fragment thereof comprising the CDRs of a HCVR and the CDRs of a LCVR, wherein the HCVR and LCVR each has an amino acid sequence selected from the HCVR and LCVR sequences listed in Table 1. In certain embodiments, the present invention provides antibodies and antigen-binding fragments thereof that bind to the same epitope as a reference antibody or antigen-binding fragment thereof comprising the CDRs of a HCVR and the CDRs of a LCVR, wherein the HCVR/LCVR amino acid sequence pair has SEQ ID NOs: 418/426.

The present invention also includes anti-LAG3 antibodies that interact with one or more amino acids contained within the extracellular domain of human LAG3 (SEQ ID NO: 588). In certain embodiments, the present invention provides anti-LAG3 antibodies and antigen-binding fragments thereof that interact with an amino acid sequence selected from the group consisting of (a) amino acids 28 to 69 of SEQ ID NO: 588; (b) amino acids 28 to 71 of SEQ ID NO: 588; (c) amino acids 31 to 52 of SEQ ID NO: 588; and (d) amino acids 32 to 69 of SEQ ID NO: 588. In certain embodiments, the present invention provides anti-LAG3 antibodies and antigen-binding fragments thereof that interact with one or more amino acids contained within SEQ ID NO: 589, for example, the present invention provides anti-LAG3 antibodies and antigen-binding fragments thereof that interact with at least 5 amino acids, at least 10 amino acids, or at least 20 amino acids contained within SEQ ID NO: 589. In certain embodiments, the present invention provides anti-LAG3 antibodies and antigen-binding fragments thereof that interact with the amino acid sequence of SEQ ID NO: 589 (corresponding to amino acids 28 to 71 of SEQ ID NO: 588).

In one embodiment, the invention provides a recombinant human monoclonal antibody or antigen-binding fragment that has one or more of the following characteristics: (a) binds specifically to human LAG3 and/or cynomolgus LAG3; (b) blocks the binding of LAG3 to MHC class II; (c) blocks LAG3-induced T cell down regulation and rescues T cell signaling; and (d) suppresses tumor growth and increases survival in a subject with cancer.

In some embodiments, the antibody or antigen binding fragment thereof may bind specifically to LAG3 in an agonist manner, i.e., it may enhance or stimulate LAG3 binding and/or activity; in other embodiments, the antibody may bind specifically to LAG3 in an antagonist manner, i.e., it may block LAG3 from binding to its ligand.

In certain embodiments, the antibodies or antigen-binding fragments of the present invention are bispecific comprising a first binding specificity to LAG3 and a second binding specificity for a second target epitope. The second target epitope may be another epitope on LAG3 or on a different protein. In certain embodiments, the second target epitope may be on a different cell including a different T cell, a B-cell, a tumor cell or a virally infected cell.

In a second aspect, the present invention provides nucleic acid molecules encoding anti-LAG3 antibodies or portions thereof. For example, the present invention provides nucleic acid molecules encoding any of the HCVR amino acid sequences listed in Table 1; in certain embodiments the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto.

The present invention also provides nucleic acid molecules encoding any of the LCVR amino acid sequences listed in Table 1; in certain embodiments the nucleic acid molecule comprises a polynucleotide sequence selected from any of the LCVR nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR1 amino acid sequences listed in Table 1; in certain embodiments the nucleic acid molecule comprises a polynucleotide sequence selected from any of the HCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR2 amino acid sequences listed in Table 1; in certain embodiments the nucleic acid molecule comprises a polynucleotide sequence selected from any of the HCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR3 amino acid sequences listed in Table 1; in certain embodiments the nucleic acid molecule comprises a polynucleotide sequence selected from any of the HCDR3 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR1 amino acid sequences listed in Table 1; in certain embodiments the nucleic acid molecule comprises a polynucleotide sequence selected from any of the LCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR2 amino acid sequences listed in Table 1; in certain embodiments the nucleic acid molecule comprises a polynucleotide sequence selected from any of the LCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR3 amino acid sequences listed in Table 1; in certain embodiments the nucleic acid molecule comprises a polynucleotide sequence selected from any of the LCDR3 nucleic acid sequences listed in Table 2, or a substantially similar sequence thereof having at least 90%, at least 95%, at least 98% or at least 99% sequence identity thereto.

The present invention also provides nucleic acid molecules encoding an HCVR, wherein the HCVR comprises a set of three CDRs (i.e., HCDR1-HCDR2-HCDR3), wherein the HCDR1-HCDR2-HCDR3 amino acid sequence set is as defined by any of the exemplary anti-LAG3 antibodies listed in Table 1.

The present invention also provides nucleic acid molecules encoding an LCVR, wherein the LCVR comprises a set of three CDRs (i.e., LCDR1-LCDR2-LCDR3), wherein the LCDR1-LCDR2-LCDR3 amino acid sequence set is as defined by any of the exemplary anti-LAG3 antibodies listed in Table 1.

The present invention also provides nucleic acid molecules encoding both an HCVR and an LCVR, wherein the HCVR comprises an amino acid sequence of any of the HCVR amino acid sequences listed in Table 1, and wherein the LCVR comprises an amino acid sequence of any of the LCVR amino acid sequences listed in Table 1. In certain embodiments, the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto, and a polynucleotide sequence selected from any of the LCVR nucleic acid sequences listed in Table 2, or a substantially similar sequence thereof having at least 90%, at least 95%, at least 98% or at least 99% sequence identity thereto. In certain embodiments according to this aspect of the invention, the nucleic acid molecule encodes an HCVR and LCVR, wherein the HCVR and LCVR are both derived from the same anti-LAG3 antibody listed in Table 1.

The present invention provides nucleic acid molecules encoding any of the heavy chain amino acid sequences listed in Table 3. The present invention also provides nucleic acid molecules encoding any of the light chain amino acid sequences listed in Table 3.

The present invention also provides nucleic acid molecules encoding both heavy chain (HC) and a light chain (LC), wherein the HC comprises an amino acid sequence of any of the HC amino acid sequences listed in Table 3, and wherein the LC comprises an amino acid sequence of any of the LC amino acid sequences listed in Table 3.

In a related aspect, the present invention provides recombinant expression vectors capable of expressing a polypeptide comprising a heavy or light chain variable region of an anti-LAG3 antibody. For example, the present invention includes recombinant expression vectors comprising any of the nucleic acid molecules mentioned above, i.e., nucleic acid molecules encoding any of the HCVR, LCVR, and/or CDR sequences as set forth in Table 1. The present invention also provides recombinant expression vectors capable of expressing a polypeptide comprising a heavy or light chain of an anti-LAG3 antibody. For example, the present invention includes recombinant expression vectors comprising any of the nucleic acid molecules mentioned above, i.e., nucleic acid molecules encoding any of the heavy chain or light chain sequences as set forth in Table 3. Also included within the scope of the present invention are host cells into which such vectors have been introduced, as well as methods of producing the antibodies or portions thereof by culturing the host cells under conditions permitting production of the antibodies or antibody fragments, and recovering the antibodies and antibody fragments so produced.

In a third aspect, the present invention provides a pharmaceutical composition comprising a recombinant human antibody or fragment thereof which specifically binds LAG3 and a pharmaceutically acceptable carrier. In a related aspect, the invention features a composition which is a combination of an anti-LAG3 antibody and a second therapeutic agent. In one embodiment, the second therapeutic agent is any agent that is advantageously combined with an anti-LAG3 antibody. Exemplary agents that may be advantageously combined with an anti-LAG3 antibody include, without limitation, other agents that bind and/or modulate LAG3 signaling (including other antibodies or antigen-binding fragments thereof, etc.) and/or agents which do not directly bind LAG3 but nonetheless modulate immune cell activation. Additional combination therapies and co-formulations involving the anti-LAG3 antibodies of the present invention are disclosed elsewhere herein.

In a fourth aspect, the invention provides methods to modulate the immune response in a subject, the method comprising administering a therapeutically effective amount of an anti-LAG3 antibody or antigen-binding fragment thereof of the invention to the subject in need thereof. In certain embodiments, the invention provides methods to enhance the immune response in a subject, the methods comprising administering to the subject an effective amount of an antibody or fragment thereof of the invention that binds LAG3. In one embodiment, the invention provides a method to stimulate or enhance T cell activation in a subject. In certain embodiments, the invention provides methods to rescue T cell activity comprising contacting the T cell with an effective amount of an antibody of the invention such that T cell activity is rescued. In one embodiment, the invention provides methods to inhibit a T regulatory (Treg) cell in a subject, the methods comprising administering a therapeutically effective amount of an antibody or antigen-binding fragment thereof of the invention to the subject in need thereof. In certain embodiments, the subject in need thereof may suffer from a disease or disorder such as cancer or viral infection. In certain embodiments, the present invention provides methods to rescue LAG3-mediated inhibition of T cell activity comprising contacting the T cell with an effective amount of an antibody of the present invention.

In a fifth aspect, the invention provides therapeutic methods for treating a disease or disorder such as cancer or viral infection in a subject using an anti-LAG3 antibody or antigen-binding portion of an antibody of the invention, wherein the therapeutic methods comprise administering a therapeutically effective amount of a pharmaceutical composition comprising an antibody or fragment of an antibody of the invention to the subject in need thereof. The disorder treated is any disease or condition which is improved, ameliorated, inhibited or prevented by stimulation or inhibition of LAG3 activity or signaling. In certain embodiments, the antibody or antigen-binding fragment thereof the invention is administered in combination with a second therapeutic agent to the subject in need thereof. The second therapeutic agent may be selected from the group consisting of an antibody to another T cell co-inhibitor, an antibody to a tumor cell antigen, an antibody to a T cell receptor, an antibody to an epitope on a virally infected cell, a cytotoxic agent, an anti-cancer drug, an anti-viral drug, an anti-inflammatory drug (e.g., corticosteroids), chemotherapeutic agent, radiation therapy, an immunosuppressant and any other drug or therapy known in the art. In certain embodiments, the second therapeutic agent may be an agent that helps to counteract or reduce any possible side effect(s) associated with an antibody or antigen-binding fragment thereof of the invention, if such side effect(s) should occur.

In certain embodiments, the present invention provides methods for suppressing tumor growth. For example, the present invention provides to suppress tumor growth due to a primary tumor or a metastatic tumor in a subject. In certain embodiments, the present invention provides methods to enhance survival (e.g., progression-free survival or overall survival) of a subject with cancer. Examples of cancer include, but are not limited to, primary and/or recurrent cancer, including blood cancer (e.g., a hematologic malignancy such as lymphoma, myeloma or leukemia), brain cancer (e.g., glioblastoma multiforme), lung cancer (e.g., non-small cell lung cancer), squamous cell carcinoma of head and neck, hepatic cell carcinoma, renal cell carcinoma, melanoma, mesothelioma, ovarian cancer, bladder cancer, breast cancer, bone cancer, colorectal cancer, prostate cancer, and colon cancer. In certain embodiments, the present invention provides methods for inhibiting or suppressing growth of established tumors. The methods comprise administering to a subject in need thereof a pharmaceutical composition comprising a therapeutically effective amount of an anti-LAG3 antibody of the present invention. In certain embodiments, the antibody is administered in combination with a second therapeutic agent selected from the group consisting of a programmed death-1 (PD-1) inhibitor (e.g., an anti-PD-1 antibody such as nivolumab or REGN2810), a programmed death-ligand 1 (PD-L1) inhibitor (e.g., an anti-PD-L1 antibody), a vascular endothelial growth factor (VEGF) antagonist (e.g., aflibercept, bevacizumab), an angiopoietin-2 (Ang2) inhibitor (e.g., an anti-Ang2 antibody such as nesvacumab), a cytotoxic T-lymphocyte antigen 4 (CTLA-4) inhibitor (e.g., ipilimumab), CD20×CD3 bispecific antibody, a cytotoxin, a chemotherapeutic agent, and radiation therapy. Additional examples of additional therapies/therapeutic agents that can be used in combination with an anti-LAG3 antibody of the invention for use in treating cancer are described elsewhere herein.

The antibody or fragment thereof may be administered subcutaneously, intravenously, intradermally, intraperitoneally, orally, intramuscularly, or intracranially. The antibody or fragment thereof may be administered at a dose of about 0.1 mg/kg of body weight to about 100 mg/kg of body weight of the subject.

The present invention also includes use of an anti-LAG3 antibody or antigen-binding fragment thereof of the invention in the manufacture of a medicament for the treatment of a disease or disorder that would benefit from the blockade of LAG3 binding and/or signaling such as cancer.

Other embodiments will become apparent from a review of the ensuing detailed description.

### BRIEF DESCRIPTION OF THE FIGURES

FIG. 1 is a schematic of the luciferase-based LAG3 bioassay described in Example 8 herein. Panel (A): “Bispec” mode: Inactive Jurkat derived T cells are activated by T-cell receptor (TCR) clustering through the CD3×CD20 bispecific antibody. Panel (B): “Peptide” mode: In this mode, inactive Jurkat derived T cells are activated by specific MHC/peptide complex (Ob2F3 TCR heterodimer with the MHCII protein, HLA-DR2AB, in complex with the MBP85-99 peptide). LAG3 binding to DR2 attenuates response in activated Jurkat derived T cells. Blocking LAG3 antibody rescues the response in activated Jurkat derived T cells.

FIGS. 2A-2B summarizes the results of in vivo efficacy of anti-mouse LAG3 antibody alone and in combination with anti-mouse PD-1 antibody against established Colon 26 tumors (described in Example 10). FIG. 2A. Average tumor volumes (mm³±SEM) in each treatment group were measured at multiple time points after tumor implantation. Treatment was started on day 14 when average tumor volume reached 50 mm³. Treatment days are indicated by arrows. All antibodies were administered intraperitoneally (i.p.) at 10 mg/kg. FIG. 2B. Individual tumor volumes within each treatment group were measured at day 35 post-implantation. Statistical significance was determined by one-way ANOVA with Dunnett's multiple comparison post-test (*p<0.05). FIGS. 2A-2B. Treatment groups: isotype controls rat IgG2a+ mouse IgG1 control antibody: (●); anti-mouse PD-1 antibody+ mouse IgG1 control (▴); anti-mouse LAG3 antibody+ rat IgG2a control (▪); anti-mouse PD-1 antibody+ anti-mouse LAG3 antibody (▾).

FIGS. 3A-3B summarizes the results of in vivo efficacy of anti-mouse LAG3 antibody alone and in combination with anti-mouse PD-1 antibody against established MC38 tumors (described in Example 11). FIG. 3A. Average tumor volumes (mm³±SEM) in each treatment group were measured at multiple time points after tumor implantation. Treatment was started on day 8, when average tumor volume reached 45 mm³. Treatment days are indicated by arrows.

FIG. 3B. Individual tumor volumes in each treatment group were measured on day 23 post-implantation. Statistical significance was determined by one-way ANOVA with Dunnett's multiple comparison post-test (****p<0.0001). FIGS. 3A-3B. Treatment groups: isotype controls rat IgG2a + mouse IgG1 (●), anti-mouse PD-1 antibody+ mlgG1 control (▴), anti-mouse LAG3 antibody+ rat IgG2a control (▪), and anti-mouse PD-1+ anti-mouse LAG3 antibody (▾).

FIGS. 4A-4B shows the expression levels of murine IFNγ and CD8b genes (normalized to murine cyclophilin B expression), as examined by Taqman analysis, in draining lymph nodes (DLN) (A) and spleen (B) collected at the end of the experiment (described in Example 11) from tumor-bearing mice. *P<0.05, **P<0.01, ***P<0.001.

FIGS. 5A-5B summarizes the in vivo efficacy of anti-human LAG3 antibody (“mAb1”) and anti-human PD-1 antibody (REGN2810) against MC38 tumors (described in Example 12). FIG. 5A. Average tumor volumes (mm³±SEM) in each treatment group at multiple time-points post-tumor implantation. Treatment days are indicated by arrows. FIG. 5B. Individual tumor volumes in each group were monitored for 24 days. The number of tumor-free mice in each group at day 24 is shown. FIGS. 5A-5B. Treatment groups: mAb1 (anti-hLAG3) at 25 mg/kg (▪); REGN2810 (anti-hPD-1) at 10 mg/kg (▴), and human isotype control antibody at 25 mg/kg (●).

FIGS. 6A-6C summarizes the in vivo efficacy of anti-human LAG3 antibody (“mAb1”) alone and in combination with anti-human PD-1 antibody (REGN2810) against MC38 tumors (described in Example 13). FIG. 6A. Average tumor volumes (mm³±SEM) in each treatment group at multiple time points post tumor implantation. Treatment days are indicated by arrows. FIG. 6B. Individual tumor volumes in each treatment group were measured on day 22 post-implantation. Statistical significance was determined by one-way ANOVA with Tukey's multiple comparison post-test (*p<0.05; **p<0.01) FIGS. 6A-6B. Treatment groups: mAb1 (anti-hLAG3) at 25 mg/kg (♦); REGN2810 (anti-hPD-1) at 10 mg/kg (▪); combination of mAb1 (anti-hLAG3) at 25 mg/kg and REGN2810 (anti-hPD-1) at 10 mg/kg (▴); human isotype control at 25 mg/kg (●). FIG. 6C. Percent tumor-free survival in treated mice. Statistical significance was determined by log-rank (Mantel-Cox) test (****p<0.0001). Treatment groups: mAb1 (anti-hLAG3) at 25 mg/kg (♦); REGN2810 (anti-hPD-1) at 10 mg/kg (▾); combination of mAb1 (anti-hLAG3) at 25 mg/kg and REGN2810 (anti-hPD-1) at 10 mg/kg (▴); human isotype control at 25 mg/kg (●).

FIGS. 7A-7C summarizes the in vivo efficacy of anti-human LAG3 antibody (“mAb1”) alone and in combination with anti-human PD-1 antibody (REGN2810) against MC38 tumors in a first experiment (described in Example 14). FIG. 7A. Average tumor volumes (mm³±SEM) in each treatment group at multiple time points post tumor implantation. Treatment days are indicated by arrows. FIG. 7B. Individual tumor volumes in each treatment group were measured on day 22 post-implantation. Statistical significance was determined by one-way ANOVA with Tukey's multiple comparison post-test (*p<0.05) FIG. 7C. Percent tumor-free survival in treated mice. Statistical significance was determined by log-rank (Mantel-Cox) test (*p<0.0197). Treatment groups: mAb1 (anti-hLAG3) at 25 mg/kg (♦); REGN2810 (anti-hPD-1) at 10 mg/kg ( [symbol] );REGN2810 (anti-hPD-1) at 1 mg/kg (▴); combination of mAb1 (anti-hLAG3) at 25 mg/kg and REGN2810 (anti-hPD-1) at 10 mg/kg (●); combination of mAb1 (anti-hLAG3) at 25 mg/kg and REGN2810 (anti-hPD-1) at 1 mg/kg (▾); and human isotype control at 25 mg/kg (●).

FIGS. 8A-8C summarizes the in vivo efficacy of anti-human LAG3 antibody (“mAb1”) alone and in combination with anti-human PD-1 antibody (REGN2810) against MC38 tumors in a second experiment (described in Example 14). FIG. 8A. Average tumor volumes (mm³±SEM) in each treatment group at multiple time points post tumor implantation. Treatment days are indicated by arrows. Statistical significance was determined by two-way ANOVA with Dunnett's multiple comparison test (*p<0.05). FIG. 8B. Individual tumor volumes in each treatment group as measured on day 23 post-implantation. Statistical significance was determined by one-way ANOVA with Dunnett's multiple comparison test (**p<0.01). FIG. 8C. Percent tumor-free survival in treated mice. Statistical significance was determined by log-rank (Mantel-Cox) test, with Bonferroni adjustment for multiple comparisons (***p<0.001, **p<0.01). Treatment groups: mAb1 (anti-hLAG3) at 25 mg/kg (♦); mAb1 (anti-hLAG3) at 5 mg/kg ( [symbol] ); REGN2810 (anti-hPD-1) at 10 mg/kg (▴); combination of mAb1 (anti-hLAG3) at 25 mg/kg and REGN2810 (anti-hPD-1) at 10 mg/kg (▪); combination of mAb1 (anti-hLAG3) at 5 mg/kg and REGN2810 (anti-hPD-1) at 10 mg/kg (▾); and human isotype control at 25 mg/kg (●).

FIG. 9 shows average tumor volumes (mm³±SEM) in each treatment group at multiple time points post tumor implantation in an experiment to assess efficacy of anti-human LAG3 antibody (“mAb1”) alone and in combination with anti-human PD-1 antibody (REGN2810) against established MC38 tumors (described in Example 15 herein). Treatment days are indicated by arrows.

FIG. 10 shows individual tumor volumes in each treatment group as measured on day 22 post-implantation in the experiment described in Example 15. Statistical significance was determined by one-way ANOVA with Dunnett's multiple comparison test.

FIG. 11 shows percent tumor-free survival in treated mice in the experiment described in Example 15 herein. Statistical significance was determined by log-rank (Mantel-Cox) test, with Bonferroni adjustment for multiple comparisons (**p<0.01).

FIG. 12 shows a graph of mean (+SD) functional mAb1 concentrations in serum vs. time following a single intravenous infusion of mAb1 in female cynomolgus monkey (described in Example 16).

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are now described. All publications mentioned herein are incorporated herein by reference in their entirety.

The term “LAG3” refers to the lymphocyte activation gene-3 protein, an immune checkpoint receptor or T cell co-inhibitor, also known as CD223. The amino acid sequence of full-length LAG3 is provided in GenBank as accession number NP_002277.4 and is also referred to herein as SEQ ID NO: 582. The term “LAG3” also includes protein variants of LAG3 having the amino acid sequence of SEQ ID NOs: 574, 575 or 576. The term “LAG3” includes recombinant LAG3 or a fragment thereof. The term also encompasses LAG3 or a fragment thereof coupled to, for example, histidine tag, mouse or human Fc, or a signal sequence such as ROR1. For example, the term includes sequences exemplified by SEQ ID NO: 575, comprising a mouse Fc (mlgG2a) at the C-terminal, coupled to amino acid residues 29-450 of full-length LAG3. Protein variants as exemplified by SEQ ID NO: 574 comprise a histidine tag at the C-terminal, coupled to amino acid residues 29-450 of full length LAG3. Unless specified as being from a non-human species, the term “LAG3” means human LAG3.

LAG3 is a member of the immunoglobulin (Ig) superfamily. LAG3 is a 503-amino acid type-1 transmembrane protein with four extracellular Ig-like domains D1 to D4 and is expressed on activated T cells, natural killer cells, B cells, plasmacytoid dendritic cells, and regulatory T cells. The LAG3 receptor binds to MHC class II molecules present on antigen presenting cells (APCs).

As used herein, the term “T cell co-inhibitor” refers to a ligand and/or receptor which modulates the immune response via T cell activation or suppression. The term “T cell co-inhibitor”, also known as T cell co-signaling molecule, includes, but is not limited to, programmed death-1 (PD-1), cytotoxic T-lymphocyte antigen-4 (CTLA-4), B and T lymphocyte attenuator (BTLA), CD-28, 2B4, LY108, T cell immunoglobulin and mucin 3(TIM3), T cell immunoreceptor with immunoglobulin and ITIM (TIGIT; also known as VSIG9), leucocyte associated immunoglobulin-like receptor 1 (LAIR1; also known as CD305), inducible T cell costimulator (ICOS; also known as CD278), V-domain Ig suppressor of T cell activation (VISTA) and CD160.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et aL. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et aL. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human anti-LAG3 monoclonal antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L}domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes fully human anti-LAG3 monoclonal antibodies comprising variants of any of the HCVR, LCVR, and/or CDR amino acid sequences disclosed herein having one or more conservative substitutions. For example, the present invention includes anti-LAG3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “recombinant”, as used herein, refers to antibodies or antigen-binding fragments thereof of the invention created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

The term “multi-specific antigen-binding molecules”, as used herein refers to bispecific, tri-specific or multi-specific antigen-binding molecules, and antigen-binding fragments thereof. Multi-specific antigen-binding molecules may be specific for different epitopes of one target polypeptide or may contain antigen-binding domains specific for epitopes of more than one target polypeptide. A multi-specific antigen-binding molecule can be a single multifunctional polypeptide, or it can be a multimeric complex of two or more polypeptides that are covalently or non-covalently associated with one another. The term “multi-specific antigen-binding molecules” includes antibodies of the present invention that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present invention, the term “multi-specific antigen-binding molecules” also includes bi-specific, tri-specific or multi-specific antibodies or antigen-binding fragments thereof. In certain embodiments, an antibody of the present invention is functionally linked to another antibody or antigen-binding fragment thereof to produce a bispecific antibody with a second binding specificity. Bispecific and multi-specific antibodies of the present invention are described elsewhere herein.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to LAG3. Moreover, multi-specific antibodies that bind to one domain in LAG3 and one or more additional antigens or a bi-specific that binds to two different regions of LAG3 are nonetheless considered antibodies that “specifically bind”, as used herein.

The term “high affinity” antibody refers to those mAbs having a binding affinity to LAG3, expressed as K_{D}, of at least 10⁻⁸ M; preferably 10⁻⁹ M; more preferably 10⁻¹⁰M, even more preferably 10⁻¹¹ M, even more preferably 10⁻¹ M, as measured by surface plasmon resonance, e.g., BIACORE™ or solution-affinity ELISA.

By the term “slow off rate”, “Koff” or “kd” is meant an antibody that dissociates from LAG3, with a rate constant of 1×10⁻³ s⁻¹ or less, preferably 1×10⁻⁴ s⁻¹ or less, as determined by surface plasmon resonance, e.g., BIACORE™.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to LAG3.

In specific embodiments, antibody or antibody fragments of the invention may be conjugated to a moiety such a ligand or a therapeutic moiety (“immunoconjugate”), such as a cytotoxin, a second anti-LAG3 antibody, an antibody to a tumor-specific antigen, an anti-cancer drug, or any other therapeutic moiety useful for treating a disease or condition including cancer or viral infection including chronic viral infection.

An “isolated antibody”, as used herein, is intended to refer to an antibody that is substantially free of other antibodies (Abs) having different antigenic specificities (e.g., an isolated antibody that specifically binds LAG3, or a fragment thereof, is substantially free of Abs that specifically bind antigens other than LAG3.

A “blocking antibody” or a “neutralizing antibody”, as used herein (or an “antibody that neutralizes LAG3 activity” or “antagonist antibody”), is intended to refer to an antibody whose binding to LAG3 results in inhibition of at least one biological activity of LAG3. For example, an antibody of the invention may prevent or block LAG3 binding to MHC class II.

An “activating antibody” or an “enhancing antibody”, as used herein (or an “agonist antibody”), is intended to refer to an antibody whose binding to LAG3 results in increasing or stimulating at least one biological activity of LAG3. For example, an antibody of the invention may increase LAG3 binding to MHC class II.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D} ”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids.

In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “cross-competes”, as used herein, means an antibody or antigen-binding fragment thereof binds to an antigen and inhibits or blocks the binding of another antibody or antigen-binding fragment thereof. The term also includes competition between two antibodies in both orientations, i.e., a first antibody that binds and blocks binding of second antibody and vice-versa. In certain embodiments, the first antibody and second antibody may bind to the same epitope. Alternatively, the first and second antibodies may bind to different, but overlapping epitopes such that binding of one inhibits or blocks the binding of the second antibody, e.g., via steric hindrance. Cross-competition between antibodies may be measured by methods known in the art, for example, by a real-time, label-free bio-layer interferometry assay. Cross-competition between two antibodies may be expressed as the binding of the second antibody that is less than the background signal due to self-self binding (wherein first and second antibodies is the same antibody). Cross-competition between 2 antibodies may be expressed, for example, as % binding of the second antibody that is less than the baseline self-self background binding (wherein first and second antibodies is the same antibody).

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

Sequence identity can be calculated using an algorithm, for example, the Needleman Wunsch algorithm (Needleman and Wunsch 1970, J. Mol. Biol. 48: 443-453) for global alignment, or the Smith Waterman algorithm (Smith and Waterman 1981, J. Mol. Biol. 147: 195-197) for local alignment. Another preferred algorithm is described by Dufresne et al in Nature Biotechnology in 2002 (vol. 20, pp. 1269-71) and is used in the software GenePAST (GQ Life Sciences, Inc. Boston, MA).

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et aL. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul etaL. (1990) J. Mol. Biol. 215: 403-410 and (1997) Nucleic Acids Res. 25:3389-3402, each of which is herein incorporated by reference.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

As used herein, the term “subject” refers to an animal, preferably a mammal, in need of amelioration, prevention and/or treatment of a disease or disorder such as viral infection, or cancer.

The term includes human subjects who have or are at risk of having cancer, metastatic cancer or viral infection.

As used herein, “anti-cancer drug” means any agent useful to treat or ameliorate or inhibit cancer including, but not limited to, cytotoxins and agents such as antimetabolites, alkylating agents, anthracyclines, antibiotics, antimitotic agents, procarbazine, hydroxyurea, asparaginase, corticosteroids, cyclophosphamide, mytotane (0,P′-(DDD)), biologics (e.g., antibodies and interferons) and radioactive agents. As used herein, “a cytotoxin or cytotoxic agent”, also refers to a chemotherapeutic agent and means any agent that is detrimental to cells. Examples include Taxol® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

As used herein, the term “anti-viral drug” refers to any drug or therapy used to treat, prevent, or ameliorate a viral infection in a host subject. The term “anti-viral drug” includes, but is not limited to zidovudine, lamivudine, abacavir, ribavirin, lopinavir, efavirenz, cobicistat, tenofovir, rilpivirine, analgesics and corticosteroids. In the context of the present invention, the viral infections include long-term or chronic infections caused by viruses including, but not limited to, human immunodeficiency virus (HIV), hepatitis B virus (HBV), hepatitis C virus (HCV), human papilloma virus (HPV), lymphocytic choriomeningitis virus (LCMV), and simian immunodeficiency virus (SIV).

As used herein, the term “to enhance immune response”, refers to an increase in activity of an immune cell such as T cell or NK cell against a tumor cell or a virally infected cell. In the context of the present invention, the term includes blocking of LAG3-mediated inhibition of T cell activity, or rescue or reversal of exhausted state of T cells. It also includes inhibition of regulatory T cell activity. The enhanced immune response, as used in the context of the present invention, results in increased killing of tumor cells and/or inhibition of tumor growth.

The antibodies and antigen-binding fragments of the present invention specifically bind to LAG3 and enhance T cell activation. The anti-LAG3 antibodies may bind to LAG3 with high affinity or with low affinity. In certain embodiments, the antibodies of the present invention may be blocking antibodies wherein the antibodies may bind to LAG3 and inhibit LAG3 signaling. In some embodiments, the antibodies of the invention block the binding of LAG3 to MHC class II and/or stimulate or enhance T cell activation. In some embodiments, the antibodies bind to LAG3 and reverse the anergic state of exhausted T cells. In certain embodiments, the antibodies bind to LAG3 and inhibit regulatory T cell activity. In some embodiments, the antibodies may be useful for stimulating or enhancing the immune response and/or for treating a subject suffering from cancer, or a viral infection. The antibodies when administered to a subject in need thereof may reduce chronic infection by a virus such as HIV, LCMV or HBV in the subject. They may be used to inhibit the growth of tumor cells in a subject. They may be used alone or as adjunct therapy with other therapeutic moieties or modalities known in the art for treating cancer, or viral infection.

In certain embodiments, the anti-LAG3 antibodies may be multi-specific antigen-binding molecules, wherein they comprise a first binding specificity to LAG3 and a second binding specificity to an antigen selected from the group consisting of another T cell co-inhibitor, and a different epitope of LAG3.

An immunogen comprising any one of the following can be used to generate antibodies to LAG3. In certain embodiments, the antibodies of the invention are obtained from mice immunized with a full length, native LAG3 (See NCBI accession number NP_002277.4) (SEQ ID NO: 582), or with a recombinant LAG3 peptide. Alternatively, LAG3 or a fragment thereof may be produced using standard biochemical techniques and modified (SEQ ID NOs: 574-576) and used as immunogen.

In certain embodiments, the immunogen is one or more extracellular domains of LAG3. In one embodiment of the invention, the immunogen is a fragment of LAG3 that ranges from about amino acid residues 29-450 of SEQ ID NO: 582.

In some embodiments, the immunogen may be a recombinant LAG3 peptide expressed in E. colior in any other eukaryotic or mammalian cells such as Chinese hamster ovary (CHO) cells.

In certain embodiments, antibodies that bind specifically to LAG3 may be prepared using fragments of the above-noted regions, or peptides that extend beyond the designated regions by about 5 to about 20 amino acid residues from either, or both, the N or C terminal ends of the regions described herein. In certain embodiments, any combination of the above-noted regions or fragments thereof may be used in the preparation of LAG3 specific antibodies.

The peptides may be modified to include addition or substitution of certain residues for tagging or for purposes of conjugation to carrier molecules, such as, KLH. For example, a cysteine may be added at either the N terminal or C terminal end of a peptide, or a linker sequence may be added to prepare the peptide for conjugation to, for example, KLH for immunization.

Certain anti-LAG3 antibodies of the present invention are able to bind to and neutralize the activity of LAG3, as determined by in vitro or in vivo assays. The ability of the antibodies of the invention to bind to and neutralize the activity of LAG3 may be measured using any standard method known to those skilled in the art, including binding assays, or activity assays, as described herein.

Non-limiting, exemplary in vitro assays for measuring binding activity are illustrated in Examples herein. In Example 3, the binding affinities and kinetic constants of human anti-LAG3 antibodies for human LAG3 were determined by surface plasmon resonance and the measurements were conducted on a Biacore 4000 or T200 instrument. In Example 4, blocking assays were used to determine cross-competition between anti-LAG3 antibodies. Examples 5 and 6 describe the binding of the antibodies to cells overexpressing LAG3. In Example 7, binding assays were used to determine the ability of the anti-LAG3 antibodies to block MHC class II-binding ability of LAG3 in vitro. In Example 8, a luciferase assay was used to determine the ability of anti-LAG3 antibodies to antagonize LAG3 signaling in T cells. In Example 9, a fluorescence assay was used to determine the ability of anti-LAG3 antibodies to bind to activated monkey CD4+ and CD8+ cells.

In certain embodiments, the antibodies of the present invention are able to enhance or stimulate T cell activity in vitro, in a subject with cancer or in a subject infected with a virus such as LCMV. In certain embodiments, the antibodies of the present invention are used in combination with a second therapeutic agent, such as an antibody to a second T cell co-inhibitor, to enhance the immune response and inhibit tumor growth in a subject.

The antibodies specific for LAG3 may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface. In one embodiment, the label may be a radionuclide, a fluorescent dye or a MRI-detectable label. In certain embodiments, such labeled antibodies may be used in diagnostic assays including imaging assays.

### Exemplary Embodiments of the Invention

In certain embodiments, the present invention provides an isolated antibody or antigen-binding fragment thereof that binds specifically to human lymphocyte activation gene 3 (LAG3) protein, wherein the antibody or antigen-binding fragment thereof has a property selected from the group consisting of: (a) binds monomeric human LAG3 with a binding dissociation equilibrium constant (K_{D}) of less than about 10 nM as measured in a surface plasmon resonance assay at 25° C.; (b) binds monomeric human LAG3 with a K_{D} less than about 8 nM as measured in a surface plasmon resonance assay at 37° C.; (c) binds dimeric human LAG3 with a K_{D} less than about 1.1 nM as measured in a surface plasmon resonance assay at 25° C.; (d) binds dimeric human LAG3 with a K_{D} less than about 1 nM as measured in a surface plasmon resonance assay at 37° C.; (e) binds to a hLAG3-expressing cell with an EC₅₀ less than about 8 nM as measured in a flow cytometry assay; (f) binds to a mfLAG3-expressing cell with a EC₅₀ less than about 2.3 nM as measured in a flow cytometry assay; (g) blocks binding of hLAG3 to human MHC class II with IC₅₀ less than about 32 nM as determined by a cell adherence assay; (h) blocks binding of hLAG3 to mouse MHC class with IC₅₀ less than about 30 nM as determined by a cell adherence assay; (i) blocks binding of hLAG3 to MHC class II by more than 90% as determined by a cell adherence assay; (j) rescues LAG3-mediated inhibition of T cell activity with EC₅₀ less than about 9 nM as determined in a luciferase reporter assay; and (k) binds to activated CD4+ and CD8+ T cells with EC₅₀ less than about 1.2 nM, as determined in a fluorescence assay.

In certain embodiments, the isolated antibody or antigen-binding fragment thereof of the invention comprises three heavy chain complementarity determining regions (CDRs) (HCDR1, HCDR2 and HCDR3) contained within any one of the heavy chain variable region (HCVR) sequences listed in Table 1; and three light chain CDRs (LCDR1, LCDR2 and LCDR3) contained within any one of the light chain variable region (LCVR) sequences listed in Table 1. In certain embodiments, the isolated antibody or antigen-binding fragment thereof comprises a HCVR having an amino acid sequence selected from the group consisting of HCVR sequences listed in Table 1.

In certain embodiments, the isolated antibody or antigen-binding fragment thereof comprises a LCVR having an amino acid sequence selected from the group consisting of LCVR sequences listed in Table 1.

In certain embodiments, the isolated antibody or antigen-binding fragment thereof comprises: (a) a HCDR1 domain having an amino acid sequence selected from the group consisting of SEQ ID NOs: 4, 20, 36, 52, 68, 84, 100, 116, 132, 148, 164, 180, 196, 212, 228, 244, 260, 276, 292, 308, 324, 340, 356, 372, 388, 404, 420, 436, 452, 460, 468, 476, 484, 492, 500, 508, 516, 540, and 556; (b) a HCDR2 domain having an amino acid sequence selected from the group consisting of SEQ ID NOs: 6, 22, 38, 54, 70, 86, 102, 118, 134, 150, 166, 182, 198, 214, 230, 246, 262, 278, 294, 310, 326, 342, 358, 374, 390, 406, 422, 438, 454, 462, 470, 478, 486, 494, 502, 510, 518, 542, and 558; (c) a HCDR3 domain having an amino acid sequence selected from the group consisting of SEQ ID NOs: 8, 24, 40, 56, 72, 88, 104, 120, 136, 152, 168, 184, 200, 216, 232, 248, 264, 280, 296, 312, 328, 344, 360, 376, 392, 408, 424, 440, 456, 464, 472, 480, 488, 496, 504, 512, 520, 544, and 560; (d) a LCDR1 domain having an amino acid sequence selected from the group consisting of SEQ ID NOs: 12, 28, 44, 60, 76, 92, 108, 124, 140, 156, 172, 188, 204, 220, 236, 252, 268, 284, 300, 316, 332, 348, 364, 380, 396, 412, 428, 444, 524, 532, 548, and 564; (e) a LCDR2 domain having an amino acid sequence selected from the group consisting of SEQ ID NOs: 14, 30, 46, 62, 78, 94, 110, 126, 142, 158, 174, 190, 206, 222, 238, 254, 270, 286, 302, 318, 334, 350, 366, 382, 398, 414, 430, 446, 526, 534, 550, and 566; and (f) a LCDR3 domain having an amino acid sequence selected from the group consisting of SEQ ID NOs: 16, 32, 48, 64, 80, 96, 112, 128, 144, 160, 176, 192, 208, 224, 240, 256, 272, 288, 304, 320, 336, 352, 368, 384, 400, 416, 432, 448, 528, 536, 552, and 568.

In certain embodiments, the isolated antibody or antigen-binding fragment thereof of the present invention comprises a HCVR/LCVR amino acid sequence pair selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/522, 458/522, 466/522, 474/522, 482/522, 490/522, 498/530, 506/530, 514/530, 538/546, and 554/562.

In certain embodiments, the isolated antibody or antigen-binding fragment of claim 7, wherein the antibody or antigen-binding fragment thereof comprises an HCVR/LCVR amino acid sequence pair selected from the group consisting of SEQ ID NOs: 386/394, 418/426, and 538/546.

In certain embodiments, the present invention provides an isolated antibody or antigen-binding fragment thereof that blocks LAG3 binding to MHC class II comprising three CDRs of a HCVR, wherein the HCVR has an amino acid sequence selected from the group consisting of SEQ ID NOs: 2, 18, 34, 50, 66, 82, 98, 114, 130, 146, 162, 178, 194, 210, 226, 242, 258, 274, 290, 306, 322, 338, 354, 370, 386, 402, 418, 434, 450, 458, 466, 474, 482, 490, 498, 506, 514, 538, and 554; and three CDRs of a LCVR, wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 522, 530, 546, and 562.

In certain embodiments, the isolated antibody or antigen-binding fragment comprises a HCVR/LCVR amino acid sequence pair selected from the group consisting of SEQ ID NOs: 386/394, 418/426, and 538/546.

In certain embodiments, the present invention provides an antibody or antigen-binding fragment thereof that competes for binding to an antibody or antigen-binding fragment thereof comprising a HCVR/LCVR amino acid sequence pair selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/522, 458/522, 466/522, 474/522, 482/522, 490/522, 498/530, 506/530, 514/530, 538/546, and 554/562.

In certain embodiments, the present invention provides an antibody or antigen-binding fragment thereof that binds to the same epitope as an antibody or antigen-binding fragment thereof comprising a HCVR/LCVR amino acid sequence pair selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/522, 458/522, 466/522, 474/522, 482/522, 490/522, 498/530, 506/530, 514/530, 538/546, and 554/562.

In certain embodiments, the present invention provides an antibody or antigen-binding fragment hereof that is a human, humanized or a chimeric antibody. Said antibody or antigen-binding fragment thereof can for instance be an IgG1 or an IgG4 antibody, such as e.g., a human IgG1 or an IgG4 antibody. The constant regions of those antibodies might correspond to wild-type constant regions, or to constant regions into which mutations have been introduced.

In certain embodiments, the present invention provides an isolated antibody comprising a heavy chain and a light chain, wherein the heavy chain comprises an amino acid sequence selected from the group consisting of SEQ ID NOs: 577, 579, and 580. In certain embodiments, the present invention provides an isolated antibody comprising a heavy chain and a light chain, wherein the light chain comprises an amino acid sequence selected from the group consisting of SEQ ID NOs: 578, and 581. In certain embodiments, the present invention provides an isolated antibody or antigen-binding fragment thereof comprising a heavy chain/light chain amino acid sequence pair selected from the group consisting of SEQ ID NOs: 577/578, 579/578, and 580/581.

In one aspect, the present invention provides a multi-specific antigen-binding molecule comprising a first antigen-binding specificity that binds specifically to LAG3 and a second antigen-binding specificity that specifically binds to a second target epitope.

In one aspect, the present invention provides a pharmaceutical composition comprising an anti-LAG3 antibody or antigen-binding fragment thereof of any of the above embodiments and a pharmaceutically acceptable carrier or diluent.

In one aspect, the present invention provides isolated polynucleotide molecules and vectors comprising polynucleotide sequences of the antibodies or antigen-binding fragment thereof disclosed herein. In certain embodiments, comprising the present invention provides an isolated polynucleotide molecule and/or a vector comprising a polynucleotide sequence that encodes a HCVR of an antibody as set forth herein. In certain embodiments, the present invention provides an isolated polynucleotide molecule and/or a vector comprising a polynucleotide sequence that encodes a LCVR of an antibody as set forth herein.

In one aspect, the present invention provides methods of enhancing an immune response in a subject, the method comprising administering a pharmaceutical composition comprising an isolated anti-LAG3 antibody or antigen-binding fragment thereof as disclosed herein. In certain embodiments, the present invention provides methods of inhibiting a T-regulatory (Treg) cell in a subject comprising administering a pharmaceutical composition comprising an isolated anti-LAG3 antibody or antigen-binding fragment thereof as disclosed herein. In certain embodiments, the present invention provides methods of enhancing T cell activation in a subject, the method comprising administering a pharmaceutical composition comprising an isolated anti-LAG3 antibody or antigen-binding fragment thereof as disclosed herein. In certain embodiments, the subject has a disease or disorder selected from the group consisting of blood cancer, brain cancer, renal cell carcinoma (e.g., clear cell renal carcinoma), ovarian cancer, bladder cancer, prostate cancer, breast cancer (e.g., triple negative breast cancer), hepatic cell carcinoma, bone cancer, colon cancer, non-small-cell lung cancer, squamous cell carcinoma of head and neck, colorectal cancer, mesothelioma, lymphoma (e.g., B cell lymphoma, diffuse large B cell lymphoma) and melanoma. In certain embodiments, the subject has a chronic viral infection caused by a virus selected from the group consisting of human immunodeficiency virus (HIV), hepatitis C virus (HCV), hepatitis B virus (HBV), human papilloma virus (HPV), lymphocytic choriomeningitis virus (LCMV) and simian immunodeficiency virus (SIV). In certain embodiments, the anti-LAG3 antibody is administered to the subject in combination with a second therapeutic agent selected from the group consisting of a PD-1 inhibitor, a CTLA inhibitor, an antibody to a tumor specific antigen, an antibody to a virally-infected-cell antigen, a PD-L1 inhibitor, a CD20 inhibitor, a bispecific antibody against CD20 and CD3, a dietary supplement such as an antioxidant, a VEGF antagonist, a chemotherapeutic agent, a cytotoxic agent, radiation, a NSAID, a corticosteroid, and any other therapy useful for ameliorating at least one symptom associated with the disease or disorder.

In one aspect, the present invention provides methods of inhibiting growth of a tumor or a tumor cell in a subject comprising administering to the subject in need thereof a therapeutically effective amount of an anti-LAG3 antibody or antigen-binding fragment thereof as disclosed herein. In certain embodiments, the tumor is primary or recurrent. In certain embodiments, the tumor is an established tumor. In certain embodiments, the subject has metastatic disease and/or has been treated with prior therapy. In certain embodiments, the tumor is present in a subject with a disease or disorder selected from the group consisting of blood cancer, brain cancer, renal cell cancer, ovarian cancer, bladder cancer, prostate cancer, breast cancer, hepatic cell carcinoma, bone cancer, colon cancer, non-small-cell lung cancer, squamous cell carcinoma of head and neck, colorectal cancer, mesothelioma, lymphoma, and melanoma. In certain embodiments, the anti-LAG3 antibody or antigen-binding fragment thereof is administered as one or more doses wherein each dose is administered 1 to 4 weeks after the immediately preceding dose. In certain embodiments, the anti-LAG3 antibody or antigen-binding fragment thereof is administered at a dose of about 0.1 mg/kg of body weight to about 100 mg/kg of body weight of the subject. In certain embodiments, the anti-LAG3 antibody is administered to the subject in combination with a second therapeutic agent selected from the group consisting of a PD-1 inhibitor, a CTLA inhibitor, an antibody to a tumor specific antigen, a PD-L1 inhibitor, a CD20 inhibitor, a bispecific antibody against CD20 and CD3, a dietary supplement such as an antioxidant, a VEGF antagonist, a chemotherapeutic agent, a cytotoxic agent, radiation, a NSAID, a corticosteroid, and any other therapy useful for ameliorating at least one symptom associated with the disease or disorder. In one embodiment, the second therapeutic agent is a PD-1 inhibitor wherein the PD-1 inhibitor is an antibody or antigen-binding fragment thereof that specifically binds to PD-1. In one embodiment, the PD-1 inhibitor is REGN2810. In certain embodiments, the anti-LAG3 antibody or antigen-binding fragment thereof is administered subcutaneously, intravenously, intradermally, intraperitoneally, orally, intramuscularly or intracranially.

In one aspect, the present invention provides methods of rescuing LAG3-mediated inhibition of T cell activity comprising contacting the T cell with an anti-LAG3 antibody or antigen-binding fragment thereof as disclosed herein. In on embodiment, the T cell is contacted by an anti-LAG3 antibody of the present invention in combination with an anti-PD-1 antibody (e.g., REGN2810).

### Antigen-Binding Fragments of Antibodies

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to LAG3. An antibody fragment may include a Fab fragment, a F(ab′)₂ fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. In such embodiments, the term “antigen-binding fragment” includes, e.g., a MHC class II molecule which binds specifically to LAG3. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (iX) V_{L}-C_{H}2; (X) V_{L}-C_{H}3; (Xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

### Preparation of Human Antibodies

Methods for generating human antibodies in transgenic mice are known in the art. Any such known methods can be used in the context of the present invention to make human antibodies that specifically bind to LAG3.

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to LAG3 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. As in the experimental section below, the antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

### Bioequivalents

The anti-LAG3 antibodies and antibody fragments of the present invention encompass proteins having amino acid sequences that vary from those of the described antibodies, but that retain the ability to bind LAG3. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present invention encompass sequences that comprise one or more additions, deletions, or substitutions of nucleotides when compared to the disclosed sequence, but that encode an antibody or antibody fragment that is essentially bioequivalent to an antibody or antibody fragment of the invention.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Anti-LAG3 Antibodies Comprising Fc Variants

According to certain embodiments of the present invention, anti-LAG3 antibodies are provided comprising an Fc domain comprising one or more mutations which enhance or diminish antibody binding to the FcRn receptor, e.g., at acidic pH as compared to neutral pH. For example, the present invention includes anti-LAG3 antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 2500 and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-LAG3 antibodies comprising an Fc domain comprising one or more pairs or groups of mutations selected from the group consisting of: 2500 and 248L (e.g., T250Q and M248L); 252Y, 254T and 256E (e.g., M252Y, S254T and T256E); 428L and 434S (e.g., M428L and N434S); 2571 and 3111 (e.g., P2571 and Q3111); 2571 and 434H (e.g., P2571 and N434H); 376V and 434H (e.g., D376V and N434H); 307A, 380A and 434A (e.g., T307A, E380A and N434A); and 433K and 434F (e.g., H433K and N434F). In one embodiment, the present invention includes anti-LAG3 antibodies comprising an Fc domain comprising a S1 08P mutation in the hinge region of IgG4 to promote dimer stabilization. All possible combinations of the foregoing Fc domain mutations, and other mutations within the antibody variable domains disclosed herein, are contemplated within the scope of the present invention.

The present invention also includes anti-LAG3 antibodies comprising a chimeric heavy chain constant (C_{H}) region, wherein the chimeric C_{H} region comprises segments derived from the C_{H} regions of more than one immunoglobulin isotype. For example, the antibodies of the invention may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the invention comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., US Patent Publication No. 20140243504, the disclosure of which is hereby incorporated by reference in its entirety). In certain embodiments, the Fc region comprises a sequence selected from the group consisting of SEQ ID NOs: 569, 570, 571, 572 and 573.

### Biological Characteristics of the Antibodies

In general, the antibodies of the present invention function by binding to LAG3. The present invention includes anti-LAG3 antibodies and antigen-binding fragments thereof that bind soluble monomeric or dimeric LAG3 molecules with high affinity. For example, the present invention includes antibodies and antigen-binding fragments of antibodies that bind monomeric LAG3 (e.g., at 25° C. or at 37° C.) with a K_{D} of less than about 10 nM as measured by surface plasmon resonance, e.g., using the assay format as defined in Example 3 herein. In certain embodiments, the antibodies or antigen-binding fragments thereof bind monomeric LAG3 with a K_{D} of less than about 5 nM, less than about 2 nM, less than about 1 nM, less than about 0.5 nM less than about 0.1 nM, less than about 0.05 nM or less than about 0.04 nM, as measured by surface plasmon resonance, e.g., using the assay format as defined in Example 3 herein, or a substantially similar assay.

The present invention also includes antibodies and antigen-binding fragments thereof that bind dimeric LAG3 (e.g., at 25° C. or at 37° C.) with a K_{D} of less than about 1.1 nM as measured by surface plasmon resonance, e.g., using the assay format as defined in Example 3 herein. In certain embodiments, the antibodies or antigen-binding fragments thereof bind dimeric LAG3 with a K_{D} of less than about 0.5 nM, less than about 0.25 nM, less than about 0.1 nM, less than about 0.05 nM, or less than about 0.01 M, as measured by surface plasmon resonance, e.g., using the assay format as defined in Example 3 herein, or a substantially similar assay.

The present invention also includes antibodies and antigen-binding fragments thereof that bind LAG3 with a dissociative half-life (t/2) of greater than about 1.6 minutes as measured by surface plasmon resonance at 25° C. or 37° C., e.g., using an assay format as defined in Example 3 herein, or a substantially similar assay. In certain embodiments, the antibodies or antigen-binding fragments of the present invention bind LAG3 with a t/2 of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1100 minutes, as measured by surface plasmon resonance at 25° C. or 37° C., e.g., using an assay format as defined in Example 3 herein (e.g., mAb-capture or antigen-capture format), or a substantially similar assay.

The present invention also includes antibodies or antigen-binding fragments thereof that bind to a human LAG3-expressing cell with an EC₅₀ less than about 8 nM as measured by a flow cytometry assay as defined in Example 5 herein, or a substantially similar assay. In certain embodiments, the antibodies or antigen-binding fragments thereof bind to a hLAG3-expressing cell with an EC₅₀ less than about 5 nM, less than about 2 nM, less than about 1 nM, or less than about 0.5 nM, as measured by a flow cytometry assay, e.g., using the assay format in Example 5 herein, or a substantially similar assay.

The present invention also includes antibodies or antigen-binding fragments thereof that bind to a cynomolgus monkey LAG3-expressing cell with an EC₅₀ less than about 2.5 nM as measured by a flow cytometry assay as defined in Example 5 herein, or a substantially similar assay. In certain embodiments, the antibodies or antigen-binding fragments thereof bind to a mfLAG3-expressing cell with an EC₅₀ less than about 2 nM, or less than about 1 nM, as measured by a flow cytometry assay, e.g., using the assay format as defined in Example 5 herein, or a substantially similar assay.

The present invention also includes antibodies or antigen-binding fragments thereof that block LAG3 binding to MHC class II (human HLA-DR2) with an IC₅₀ of less than about 32 nM as determined using a cell adherence assay, e.g., as shown in Example 7, or a substantially similar assay. In certain embodiments, the antibodies or antigen-binding fragments thereof block LAG3 binding to human MHC class II with an IC₅₀ less than about 25 nM, less than about 20 nM, less than about 10 nM, or less than about 5 nM, as measured by a cell adherence assay, e.g., using the assay format as defined in Example 7 herein, or a substantially similar assay.

The present invention also includes antibodies or antigen-binding fragments thereof that block LAG3 binding to MHC class II (mouse HLA-DR2) with an IC₅₀ of less than about 30 nM as determined using a cell adherence assay, e.g., as shown in Example 7, or a substantially similar assay. In certain embodiments, the antibodies or antigen-binding fragments thereof block mouse LAG3 binding to human MHC class II with an IC₅₀ less than about 25 nM, less than about 20 nM, less than about 10 nM, or less than about 5 nM, as measured by a cell adherence assay, e.g., using the assay format as defined in Example 7 herein, or a substantially similar assay.

The present invention also includes antibodies or antigen-binding fragment thereof that block binding of LAG3 to human or mouse MHC class II by more than 90% as measured by a cell adherence assay as defined in Example 7 herein, or a substantially similar assay.

The present invention also includes antibodies or antigen-binding fragments thereof that block LAG-induced T cell down-regulation with an EC₅₀ less than 9 nM as measured by a T cell/APC luciferase reporter assay as defined in Example 8 herein, or a substantially similar assay. In certain embodiments, the antibodies or antigen-binding fragments thereof block LAG3-induced T cell down-regulation with an EC₅₀ less than about 5 nM, less than about 1 nM, less than about 0.5 nM, or less than about 0.1 nM, as measured by a T cell/APC luciferase reporter assay, e.g., using the assay format as defined in Example 8 herein, or a substantially similar assay.

The present invention also includes antibodies or antigen-binding fragments thereof that bind to cynomolgus activated CD4+ and CD8+ T cells with an EC₅₀ less than about 1.2 nM as measured by a fluorescence assay as defined in Example 9 herein, or a substantially similar assay.

In certain embodiments, the antibodies or antigen-binding fragments thereof bind to cynomolgus activated CD4+ and CD8+ T cells with an EC₅₀ less than about 1.1 nM, less than about 1 nM, less than about 0.5 nM, less than about 0.2 nM, or less than about 0.1 nM, as measured by a fluorescence assay, e.g., using the assay format as defined in Example 9 herein, or a substantially similar assay.

In certain embodiments, the antibodies of the present invention may function by blocking or inhibiting the MHC class II-binding activity associated with LAG3 by binding to any other region or fragment of the full length protein, the amino acid sequence of which is shown in SEQ ID NO: 582.

In certain embodiments, the antibodies of the present invention are useful in inhibiting the growth of a tumor or delaying the progression of cancer when administered prophylactically to a subject in need thereof and may increase survival of the subject. For example, the administration of an antibody of the present invention may lead to shrinking of a primary tumor and may prevent metastasis or development of secondary tumors. In certain embodiments, the antibodies of the present invention are useful in inhibiting the growth of a tumor when administered therapeutically to a subject in need thereof and may increase survival of the subject. For example, the administration of a therapeutically effective amount of an antibody of the invention to a subject may lead to shrinking and disappearance of an established tumor in the subject.

In one embodiment, the invention provides an isolated recombinant monoclonal antibody or antigen-binding fragment thereof that binds to LAG3, wherein the antibody or fragment thereof exhibits one or more of the following characteristics: (i) comprises a HCVR having an amino acid sequence selected from the group consisting of SEQ ID NO: 2, 18, 34, 50, 66, 82, 98, 114, 130, 146, 162, 178, 194, 210, 226, 242, 258, 274, 290, 306, 322, 338, 354, 370, 386, 402, 418, 434, 450, 458, 466, 474, 482, 490, 498, 506, 514, 538, and 554, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362, 378, 394, 410, 426, 442, 522, 530, 546, and 562, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 200, 216, 232, 248, 264, 280, 296, 312, 328, 344, 360, 376, 392, 408, 424, 440, 456, 464, 472, 480, 488, 496, 504, 512, 520, 544, and 560, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 112, 128, 144, 160, 176, 192, 208, 224, 240, 256, 272, 288, 304, 320, 336, 352, 368, 384, 400, 416, 432, 448, 528, 536, 552, and 568, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 116, 132, 148, 164, 180, 196, 212, 228, 244, 260, 276, 292, 308, 324, 340, 356, 372, 388, 404, 420, 436, 452, 460, 468, 476, 484, 492, 500, 508, 516, 540, and 556, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 118,134, 150, 166, 182, 198,214,230,246,262,278,294, 310, 326, 342, 358, 374, 390, 406, 422, 438, 454, 462, 470, 478, 486, 494, 502, 510, 518, 542, and 558, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 188, 204, 220, 236, 252, 268, 284, 300, 316, 332, 348, 364, 380, 396, 412, 428, 444, 524, 532, 548, and 564, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 78, 94, 110, 126, 142, 158, 174, 190, 206, 222, 238, 254, 270, 286, 302, 318, 334, 350, 366, 382, 398, 414, 430, 446, 526, 534, 550, and 566, or a substantially similar sequence thereof having at least 90%, at least 95%, at least 98% or at least 99% sequence identity; (v) binds monomeric human LAG3 with a binding dissociation equilibrium constant (K_{D}) of less than about 10 nM as measured in a surface plasmon resonance assay at 25° C.; (vi) binds monomeric human LAG3 with a K_{D} less than about 8 nM as measured in a surface plasmon resonance assay at 37° C.; (vii) binds dimeric human LAG3 with a K_{D} less than about 1.1 nM as measured in a surface plasmon resonance assay at 25° C.; (viii) binds dimeric human LAG3 with a K_{D} less than about 1 nM as measured in a surface plasmon resonance assay at 37° C.; (ix) binds to a hLAG3-expressing cell with an EC₅₀ less than about 8 nM as measured in a flow cytometry assay; (x) binds to a mfLAG3-expressing cell with a EC₅₀ less than about 2.3 nM as measured in a flow cytometry assay; (xi) blocks binding of hLAG3 to human MHC class II with IC₅₀ less than about 32 nM as determined by a cell adherence assay; (xii) blocks binding of hLAG3 to mouse MHC class with IC₅₀ less than about 30 nM as determined by a cell adherence assay; (xiii) blocks binding of hLAG3 to MHC class II by more than 90% as determined by a cell adherence assay; (xiv) rescues LAG3-mediated inhibition of T cell activity with EC₅₀ less than about 9 nM as determined in a luciferase reporter assay; (xv) binds to activated CD4+ and CD8+ T cells with EC₅₀ less than about 1.2 nM, as determined in a fluorescence assay; (xvi) suppresses tumor growth and increases survival in a subject with cancer, and (xvii) is fully human.

The antibodies of the present invention may possess one or more of the aforementioned biological characteristics, or any combinations thereof. Other biological characteristics of the antibodies of the present invention will be evident to a person of ordinary skill in the art from a review of the present disclosure including the working Examples herein.

### Species Selectivity and Species Cross-Reactivity

According to certain embodiments of the invention, the anti-LAG3 antibodies bind to human LAG3 but not to LAG3 from other species. Alternatively, the anti-LAG3 antibodies of the invention, in certain embodiments, bind to human LAG3 and to LAG3 from one or more non-human species. For example, the anti-LAG3 antibodies of the invention may bind to human LAG3 and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee LAG3. In certain embodiments, the anti-LAG3 antibodies of the invention may bind to human and cynomolgus LAG3 with the same affinities or with different affinities, but do not bind to rat and mouse LAG3.

### Epitope Mapping and Related Technologies

The present invention includes anti-LAG3 antibodies which interact with one or more amino acids found within one or more domains of the LAG3 molecule including, e.g., extracellular D1 to D4 domains, a transmembrane domain, and an intracellular domain. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within any of the aforementioned domains of the LAG3 molecule (e.g. a linear epitope in a domain). Alternatively, the epitope may consist of a plurality of non-contiguous amino acids (or amino acid sequences) located within either or both of the aforementioned domains of the LAG3 molecule (e.g. a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, NY). Other methods include alanine scanning mutational analysis, peptide blot analysis (Reineke (2004) Methods Mol. Biol. 248: 443-63), peptide cleavage analysis crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the invention into groups of antibodies binding different epitopes.

In certain embodiments, the anti-LAG3 antibodies or antigen-binding fragments thereof bind an epitope within any one or more of the regions exemplified in LAG3, either in natural form, as exemplified in SEQ ID NO: 582, or recombinantly produced, as exemplified in SEQ ID NOS: 574-576, or to a fragment thereof. In some embodiments, the antibodies of the invention bind to an extracellular region comprising one or more amino acids selected from the group consisting of amino acid residues 29-450 of LAG3. In some embodiments, the antibodies of the invention bind to an extracellular region comprising one or more amino acids selected from the group consisting of amino acid residues 1-533 of cynomolgus LAG3, as exemplified by SEQ ID NO: 576.

In certain embodiments, the present invention includes anti-LAG3 antibodies and antigen-binding fragments thereof that interact with one or more epitopes found within the extracellular region of LAG3 (SEQ ID NO: 588). The epitope(s) may consist of one or more contiguous sequences of 3 or more (e.g., 3, 4, 5, 6, 7, 8, 9, 10, 11, 12, 13, 14, 15, 16, 17, 18, 19, 20 or more) amino acids located within the extracellular region of LAG3. Alternatively, the epitope may consist of a plurality of non-contiguous amino acids (or amino acid sequences) located within the extracellular region of LAG3. As shown in Example 18 herein, the epitope of LAG3 with which the exemplary antibody of the invention H4sH15482P interacts is defined by the amino acid sequence LRRAGVTWQHQPDSGPPAAAPGHPLAPGPHPAAPSSWGPRPRRY (SEQ ID NO: 589), which corresponds to amino acids 28 to 71 of SEQ ID NO: 588. Accordingly, the present invention includes anti-LAG3 antibodies that interact with one or more amino acids contained within the region consisting of amino acids 28 to 71 of SEQ ID NO: 588 (i.e., the sequence LRRAGVTWQHQPDSGPPAAAPGHPLAPGPHPAAPSSWGPRPRRY [SEQ ID NO: 589]).

The present invention includes anti-LAG3 antibodies that bind to the same epitope, or a portion of the epitope, as any of the specific exemplary antibodies described herein in Table 1, or an antibody having the CDR sequences of any of the exemplary antibodies described in Table 1. Likewise, the present invention also includes anti-LAG3 antibodies that compete for binding to LAG3 or a LAG3 fragment with any of the specific exemplary antibodies described herein in Table 1, or an antibody having the CDR sequences of any of the exemplary antibodies described in Table 1. For example, the present invention includes anti-LAG3 antibodies that cross-compete for binding to LAG3 with one or more antibodies as defined in Example 4 herein (e.g., H4sH15482P, H4sH15479P, H4sH14813N, H4H14813N, H4H15479P, H4H15482P, H4H15483P, H4sH15498P, H4H15498P, H4H17828P2, H4H17819P, and H4H17823P).

One can easily determine whether an antibody binds to the same epitope as, or competes for binding with, a reference anti-LAG3 antibody by using routine methods known in the art. For example, to determine if a test antibody binds to the same epitope as a reference anti-LAG3 antibody of the invention, the reference antibody is allowed to bind to a LAG3 protein or peptide under saturating conditions. Next, the ability of a test antibody to bind to the LAG3 molecule is assessed. If the test antibody is able to bind to LAG3 following saturation binding with the reference anti-LAG3 antibody, it can be concluded that the test antibody binds to a different epitope than the reference anti-LAG3 antibody. On the other hand, if the test antibody is not able to bind to the LAG3 protein following saturation binding with the reference anti-LAG3 antibody, then the test antibody may bind to the same epitope as the epitope bound by the reference anti-LAG3 antibody of the invention.

To determine if an antibody competes for binding with a reference anti-LAG3 antibody, the above-described binding methodology is performed in two orientations: In a first orientation, the reference antibody is allowed to bind to a LAG3 protein under saturating conditions followed by assessment of binding of the test antibody to the LAG3 molecule. In a second orientation, the test antibody is allowed to bind to a LAG3 molecule under saturating conditions followed by assessment of binding of the reference antibody to the LAG3 molecule. If, in both orientations, only the first (saturating) antibody is capable of binding to the LAG3 molecule, then it is concluded that the test antibody and the reference antibody compete for binding to LAG3. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Immunoconjugates

The invention encompasses a human anti-LAG3 monoclonal antibody conjugated to a therapeutic moiety (“immunoconjugate”), such as a cytotoxin or a chemotherapeutic agent to treat cancer. As used herein, the term “immunoconjugate” refers to an antibody which is chemically or biologically linked to a cytotoxin, a radioactive agent, a cytokine, an interferon, a target or reporter moiety, an enzyme, a toxin, a peptide or protein or a therapeutic agent. The antibody may be linked to the cytotoxin, radioactive agent, cytokine, interferon, target or reporter moiety, enzyme, toxin, peptide or therapeutic agent at any location along the molecule so long as it is able to bind its target. Examples of immunoconjugates include antibody drug conjugates and antibody-toxin fusion proteins. In one embodiment, the agent may be a second different antibody to LAG3. In certain embodiments, the antibody may be conjugated to an agent specific for a tumor cell or a virally infected cell. The type of therapeutic moiety that may be conjugated to the anti-LAG3 antibody and will take into account the condition to be treated and the desired therapeutic effect to be achieved. Examples of suitable agents for forming immunoconjugates are known in the art; see for example, WO 05/103081.

### Multi-specific Antibodies

The antibodies of the present invention may be mono-specific, bi-specific, or multi-specific. Multi-specific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244.

In one aspect, the present invention includes multi-specific antigen-binding molecules or antigen-binding fragments thereof wherein one specificity of an immunoglobulin is specific for the extracellular domain of LAG3, or a fragment thereof, and the other specificity of the immunoglobulin is specific for binding outside the extracellular domain of LAG3, or a second therapeutic target, or is conjugated to a therapeutic moiety.

Any of the multi-specific antigen-binding molecules of the invention, or variants thereof, may be constructed using standard molecular biological techniques (e.g., recombinant DNA and protein expression technology), as will be known to a person of ordinary skill in the art.

In some embodiments, LAG3-specific antibodies are generated in a bi-specific format (a “bi-specific”) in which variable regions binding to distinct domains of LAG3 are linked together to confer dual-domain specificity within a single binding molecule. Appropriately designed bi-specifics may enhance overall LAG3 inhibitory efficacy through increasing both specificity and binding avidity. Variable regions with specificity for individual domains, (e.g., segments of the N-terminal domain), or that can bind to different regions within one domain, are paired on a structural scaffold that allows each region to bind simultaneously to the separate epitopes, or to different regions within one domain. In one example for a bi-specific, heavy chain variable regions (V_{H}) from a binder with specificity for one domain are recombined with light chain variable regions (V_{L}) from a series of binders with specificity for a second domain to identify non-cognate V_{L} partners that can be paired with an original V_{H} without disrupting the original specificity for that V_{H}. In this way, a single V_{L} segment (e.g., V_{L}1) can be combined with two different V_{H} domains (e.g., V_{H}1 and V_{H}2) to generate a bi-specific comprised of two binding “arms” (V_{H}1-V_{L}1 and V_{H}2-V_{L}1). Use of a single V_{L}segment reduces the complexity of the system and thereby simplifies and increases efficiency in cloning, expression, and purification processes used to generate the bi-specific (See, for example, U.S. Ser. No. 13/022,759 and US2010/0331527).

Alternatively, antibodies that bind more than one domains and a second target, such as, but not limited to, for example, a second different anti-LAG3 antibody, may be prepared in a bi-specific format using techniques described herein, or other techniques known to those skilled in the art. Antibody variable regions binding to distinct regions may be linked together with variable regions that bind to relevant sites on, for example, the extracellular domain of LAG3, to confer dual-antigen specificity within a single binding molecule. Appropriately designed bi-specifics of this nature serve a dual function. Variable regions with specificity for the extracellular domain are combined with a variable region with specificity for outside the extracellular domain and are paired on a structural scaffold that allows each variable region to bind to the separate antigens.

An exemplary bi-specific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bi-specific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies. Variations on the bi-specific antibody format described above are contemplated within the scope of the present invention.

Other exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/lgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

### Therapeutic Administration and Formulations

The invention provides therapeutic compositions comprising the anti-LAG3 antibodies or antigen-binding fragments thereof of the present invention. Therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody may vary depending upon the age and the size of a subject to be administered, target disease, conditions, route of administration, and the like. When an antibody of the present invention is used for treating a disease or disorder in an adult patient, or for preventing such a disease, it is advantageous to administer the antibody of the present invention normally at a single dose of about 0.1 to about 60 mg/kg body weight, more preferably about 5 to about 60, about 20 to about 50, about 10 to about 50, about 1 to about 10, or about 0.8 to about 11 mg/kg body weight. Depending on the severity of the condition, the frequency and the duration of the treatment can be adjusted. In certain embodiments, the antibody or antigen-binding fragment thereof of the invention can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer (1990) Science 249:1527-1533).

The use of nanoparticles to deliver the antibodies of the present invention is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389, 24 pages, doi: 10.1155/2009/439389), incorporated herein by reference. Nanoparticles may be developed and conjugated to antibodies contained in pharmaceutical compositions to target tumor cells or autoimmune tissue cells or virally infected cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ 1, || and Ill (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™ OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.) and the HUMIRA™ Pen (Abbott Labs, Abbott Park, IL), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, it is preferred that the antibody is contained in about 5 to about 100 mg and in about 10 to about 250 mg for the other dosage forms.

### Therapeutic Uses of the Antibodies

The antibodies of the invention are useful, inter alia, for the treatment, prevention and/or amelioration of any disease or disorder associated with or mediated by LAG3 expression, signaling or activity, or treatable by blocking the interaction between LAG3 and the LAG3 ligand MHC class II or otherwise inhibiting LAG3 activity and/or signaling. For example, the present invention provides methods for treating cancer (tumor growth inhibition) and/or viral infections by administering an anti-LAG3 antibody (or pharmaceutical composition comprising an anti-LAG3 antibody) as described herein to a patient in need of such treatment, and anti-LAG3 antibodies (or pharmaceutical composition comprising an anti-LAG3 antibody) for use in the treatment of cancer (tumor growth inhibition) and/or viral infections. The antibodies of the present invention are useful for the treatment, prevention, and/or amelioration of disease or disorder or condition such as cancer or a viral infection and/or for ameliorating at least one symptom associated with such disease, disorder or condition. In the context of the methods of treatment described herein, the anti-LAG3 antibody may be administered as a monotherapy (i.e., as the only therapeutic agent) or in combination with one or more additional therapeutic agents (examples of which are described elsewhere herein).

In some embodiments of the invention, the antibodies described herein are useful for treating subjects suffering from primary or recurrent cancer, including, but not limited to, blood cancer, brain cancer (e.g., glioblastoma multiforme), renal cell carcinoma (e.g., clear cell renal cancer), ovarian cancer, bladder cancer, prostate cancer, breast cancer (e.g., triple negative breast cancer), hepatic cell carcinoma, bone cancer, colon cancer, non-small-cell lung cancer, squamous cell carcinoma of head and neck, colorectal cancer, mesothelioma, and melanoma.

As used herein, the term “blood cancer” includes a hematologic malignancy that affects blood, bone marrow, lymph or lymphatic system. As such, the term includes malignancies of cells from the lymphoid and myeloid cell lineages. The myeloid cell line normally produces granulocytes, erythrocytes, thrombocytes, macrophages, and mast cells; the lymphoid cell line produces B, T, NK and plasma cells. The term, therefore, includes malignancies of the above-mentioned cells, viz. lymphomas, myelomas, lymphoid leukemias and myelogenous leukemias. Examples include, but are not limited to, acute lymphoblastic leukemia, acute myelogenous leukemia, chronic lymphocytic leukemia, chronic myelogenous leukemia, acute monocytic leukemia, Hodgkin's lymphomas, non-Hodgkin's lymphomas (e.g., B cell lymphoma, diffuse large B cell lymphoma), and myeloma (including multiple myeloma).

The antibodies may be used to treat early stage or late-stage symptoms of cancer. In one embodiment, an antibody or fragment thereof of the invention may be used to treat advanced or metastatic cancer. The antibodies are useful in reducing or inhibiting or shrinking tumor growth of both solid tumors and blood cancers. In certain embodiments, treatment with an antibody or antigen-binding fragment thereof of the invention leads to more than 40% regression, more than 50% regression, more than 60% regression, more than 70% regression, more than 80% regression or more than 90% regression of a tumor in a subject. In certain embodiments, the antibodies may be used to prevent relapse of a tumor. In certain embodiments, the antibodies are useful in extending progression-free survival or overall survival in a subject with cancer. In some embodiments, the antibodies are useful in reducing toxicity due to chemotherapy or radiotherapy while maintaining long-term survival in a patient suffering from cancer.

In certain embodiments, the subject is a patient suffering from cancer, and who:
has not previously received therapy with anti-PD-1/PD-L1 but are appropriate candidates to receive anti-PD-1-based therapy; and/or
has previously received anti-PD-1/PD-L1 based therapy and had a confirmed objective response (CR or PR) or SD for at least 3 months on anti-PD-1/PD-L1 therapy but subsequently progressed on that therapy or had SD or a PR as best response with subsequent stable response for 6 months; and/or
is not candidate for standard therapy, or for whom no available therapy is expected to convey clinical benefit and are appropriate for mAb1 monotherapy; and/or
is suffering from anti-PD-1/PD-L1 naïve stage IIIB or IV NSCLC either without prior therapy for metastatic disease or with disease progression/recurrence after one platinum-containing regimen; and/or
is suffering from anti-PD-1/PD-L1 experienced stage IIIB or IV NSCLC with no more than 2 prior therapies for metastatic disease; and/or
is suffering from anti-PD-1/PD-L1 naïve advanced or metastatic ccRCC with a clear cell component who had received 1 to 2 previous regimens of anti-angiogenic therapy; and/or
is suffering from anti-PD-1/PD-L1 experienced* advanced or metastatic ccRCC with a clear cell component who had received 1 to 2 previous regimens of anti-angiogenic therapy; and/or
is suffering from anti-PD-1/PD-L1 naïve metastatic TNBC (estrogen, progesterone, and human epidermal growth factor receptor 2 negative) who have received 5 or fewer prior lines of therapy; and/or
is suffering from anti-PD-1/PD-L1 naïve advanced or metastatic melanoma who have received no more than 2 previous regimens for metastatic disease; and/or
is suffering from anti-PD-1/PD-L1 experienced advanced or metastatic melanoma who have received no more than 2 previous regimens for metastatic disease; and/or
is suffering from anti-PD-1/PD-L1 naïve relapsed/refractory DLBCL who have either progressed after or are not candidates for autologous stem cell transplant; and/or
is suffering from anti-PD-1/PD-L1 experience* relapsed/refractory DLBCL who have either progressed after or are not candidates for autologous stem cell transplant.

In certain embodiments, the antibodies of the invention are useful to treat subjects suffering from a chronic viral infection. In some embodiments, the antibodies of the invention are useful in decreasing viral titers in the host and/or rescuing exhausted T cells. In certain embodiments, an antibody or fragment thereof of the invention may be used to treat chronic viral infection by lymphocytic choriomeningitis virus (LCMV). In some embodiments, an antibody or antigen-binding fragment thereof the invention may be administered at a therapeutic dose to a patient with an infection by human immunodeficiency virus (HIV) or human papilloma virus (HPV) or hepatitis B/C virus (HBV/HCV). In a related embodiment, an antibody or antigen-binding fragment thereof of the invention may be used to treat an infection by simian immunodeficiency virus (SIV) in a simian subject such as cynomolgus.

In certain embodiments, a blocking antibody of the present invention may be administered in a therapeutically effective amount to a subject suffering from a cancer or a viral infection.

One or more antibodies of the present invention may be administered to relieve or prevent or decrease the severity of one or more of the symptoms or conditions of the disease or disorder.

It is also contemplated herein to use one or more antibodies of the present invention prophylactically to patients at risk for developing a disease or disorder such as cancer, and viral infection.

In a further embodiment of the invention, the present antibodies are used for the preparation of a pharmaceutical composition for treating patients suffering from cancer, or viral infection. In another embodiment of the invention, the present antibodies are used as adjunct therapy with any other agent or any other therapy known to those skilled in the art useful for treating cancer or viral infection.

### Combination Therapies and Formulations

Combination therapies may include an anti-LAG3 antibody of the invention and any additional therapeutic agent that may be advantageously combined with an antibody of the invention, or with a biologically active fragment of an antibody of the invention.

The antibodies of the present invention may be combined synergistically with one or more anti-cancer drugs or therapy used to treat or inhibit cancer, including, for example, blood cancer, brain cancer (e.g., glioblastoma multiforme), renal cell carcinoma, ovarian cancer, bladder cancer, prostate cancer, breast cancer, hepatic cell carcinoma, bone cancer, colon cancer, non-small-cell lung cancer, squamous cell carcinoma of head and neck, colorectal cancer, mesothelioma, and melanoma. It is contemplated herein to use anti-LAG3 antibodies of the invention in combination with immunostimulatory and/or immunosupportive therapies to inhibit tumor growth, and/or enhance survival of cancer patients. The immunostimulatory therapies include direct immunostimulatory therapies to augment immune cell activity by either “releasing the brake” on suppressed immune cells or “stepping on the gas” to activate an immune response. Examples include targeting other checkpoint receptors, vaccination and adjuvants. The immunosupportive modalities may increase antigenicity of the tumor by promoting immunogenic cell death, inflammation or have other indirect effects that promote an anti-tumor immune response. Examples include radiation, chemotherapy, anti-angiogenic agents, and surgery.

In various embodiments, one or more antibodies of the present invention may be used in combination with a PD-1 inhibitor (e.g., an anti-PD-1 antibody such as nivolumab, pembrolizumab, pidilizumab, BGB-A317 or REGN2810), a PD-L1 inhibitor (e.g., an anti-PD-L1 antibody such as avelumab, atezolizumab, durvalumab, MDX-1105, or REGN3504), a CTLA-4 inhibitor (e.g., ipilimumab), a TIM3 inhibitor, a BTLA inhibitor, a TIGIT inhibitor, a CD47 inhibitor, a GITR inhibitor, an antagonist of another T cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGF3) inhibitor, an epidermal growth factor receptor (EGFR) inhibitor (e.g., erlotinib, cetuximab), a CD20 inhibitor (e.g., an anti-CD20 antibody such as rituximab), an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], a vaccine (e.g., Bacillus Calmette-Guerin, a cancer vaccine), an adjuvant to increase antigen presentation (e.g., granulocyte-macrophage colony-stimulating factor), a bispecific antibody (e.g., CD3×CD20 bispecific antibody, or PSMAxCD3 bispecific antibody), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, an antibody-drug conjugate (ADC) (e.g., anti-CD19-DM4 ADC, and anti-DS6-DM4 ADC), an anti-inflammatory drug (e.g., corticosteroids, and non-steroidal anti-inflammatory drugs), a dietary supplement such as anti-oxidants or any other therapy care to treat cancer. In certain embodiments, the anti-LAG3 antibodies of the present invention may be used in combination with cancer vaccines including dendritic cell vaccines, oncolytic viruses, tumor cell vaccines, etc. to augment the anti-tumor response. Examples of cancer vaccines that can be used in combination with anti-LAG3 antibodies of the present invention include MAGE3 vaccine for melanoma and bladder cancer, MUC1 vaccine for breast cancer, EGFRv3 (e.g., Rindopepimut) for brain cancer (including glioblastoma multiforme), or ALVAC-CEA (for CEA+ cancers).

In certain embodiments, the anti-LAG3 antibodies of the invention may be administered in combination with radiation therapy in methods to generate long-term durable anti-tumor responses and/or enhance survival of patients with cancer. In some embodiments, the anti-LAG3 antibodies of the invention may be administered prior to, concomitantly or after administering radiation therapy to a cancer patient. For example, radiation therapy may be administered in one or more doses to tumor lesions followed by administration of one or more doses of anti-LAG3 antibodies of the invention. In some embodiments, radiation therapy may be administered locally to a tumor lesion to enhance the local immunogenicity of a patient's tumor (adjuvinating radiation) and/or to kill tumor cells (ablative radiation) followed by systemic administration of an anti-LAG3 antibody of the invention. For example, intracranial radiation may be administered to a patient with brain cancer (e.g., glioblastoma multiforme) in combination with systemic administration of an anti-LAG3 antibody of the invention. In certain embodiments, the anti-LAG3 antibodies of the invention may be administered in combination with radiation therapy and a chemotherapeutic agent (e.g., temozolomide) or a VEGF antagonist (e.g., aflibercept).

In certain embodiments, the anti-LAG3 antibodies of the invention may be administered in combination with one or more anti-viral drugs to treat chronic viral infection caused by LCMV, HIV, HPV, HBV or HCV. Examples of anti-viral drugs include, but are not limited to, zidovudine, lamivudine, abacavir, ribavirin, lopinavir, efavirenz, cobicistat, tenofovir, rilpivirine and corticosteroids.

The additional therapeutically active agent(s)/component(s) may be administered prior to, concurrent with, or after the administration of the anti-LAG3 antibody of the present invention. For purposes of the present disclosure, such administration regimens are considered the administration of an anti-LAG3 antibody “in combination with” a second therapeutically active component.

The additional therapeutically active component(s) may be administered to a subject prior to administration of an anti-LAG3 antibody of the present invention. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an anti-LAG3 antibody of the present invention. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of an anti-LAG3 antibody of the present invention. “Concurrent” administration, for purposes of the present invention, includes, e.g., administration of an anti-LAG3 antibody and an additional therapeutically active component to a subject in a single dosage form (e.g., co-formulated), or in separate dosage forms administered to the subject within about 30 minutes or less of each other. If administered in separate dosage forms, each dosage form may be administered via the same route (e.g., both the anti-LAG3 antibody and the additional therapeutically active component may be administered intravenously, subcutaneously, etc.); alternatively, each dosage form may be administered via a different route (e.g., the anti-LAG3 antibody may be administered intravenously, and the additional therapeutically active component may be administered subcutaneously). In any event, administering the components in a single dosage from, in separate dosage forms by the same route, or in separate dosage forms by different routes are all considered “concurrent administration,” for purposes of the present disclosure. For purposes of the present disclosure, administration of an anti-LAG3 antibody “prior to”, “concurrent with,” or “after” (as those terms are defined herein above) administration of an additional therapeutically active component is considered administration of an anti-LAG3 antibody “in combination with” an additional therapeutically active component).

The present invention includes pharmaceutical compositions in which an anti-LAG3 antibody of the present invention is co-formulated with one or more of the additional therapeutically active component(s) as described elsewhere herein using a variety of dosage combinations.

In exemplary embodiments in which an anti-LAG3 antibody of the invention is administered in combination with a PD-1 inhibitor (e.g., an anti-PD-1 antibody as disclosed in US 2015/0203579, herein incorporated by reference in its entirety), including administration of co-formulations comprising an anti-LAG3 antibody and a PD-1 inhibitor, the individual components may be administered to a subject and/or co-formulated using a variety of dosage combinations. Thus, the present invention includes a combination of (i) an anti-LAG3 antibody of the invention, and (ii) a PD-1 inhibitor (e.g., an anti-PD-1 antibody as disclosed in US 2015/0203579, herein incorporated by reference in its entirety), for simultaneous, separate and/or sequential use in the treatment of cancer or viral infections. For example, the anti-LAG3 antibody and the PD-1 inhibitor (e.g., an anti-PD-1 antibody) each may be administered to a subject and/or contained in a co-formulation in an amount selected from the group consisting of 0.01 mg, 0.02 mg, 0.03 mg, 0.04 mg, 0.05 mg, 0.1 mg, 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg, 3.0 mg, 3.5 mg, 4.0 mg, 4.5 mg, 5.0 mg, 6.0 mg, 7.0 mg, 8.0 mg, 9.0 mg, and 10.0 mg. The combinations/co-formulations may be administered to a subject according to any of the administration regimens disclosed elsewhere herein, including, e.g., twice a week, once every week, once every 2 weeks, once every 3 weeks, once every month, once every 2 months, once every 3 months, once every 4 months, once every 5 months, once every 6 months, etc. The anti-LAG3 antibody of the invention might, for instance, be administered at a dose of about 0.8 to about 11, about 1 to about 10, about 3 to about 10, about 1, about 3 or about 10 mg/kg, simultaneously with an PD-1 inhibitor (e.g. an anti-PD-1 antibody as disclosed in US 2015/0203579) at a dose of about 3 to 5, or about 3.0 mg/kg. The simultaneous administration might for instance occur every 14 days, 21 days or 28 days.

In exemplary embodiments in which an anti-LAG3 antibody of the invention is administered in combination with a VEGF antagonist (e.g., a VEGF trap such as aflibercept), including administration of co-formulations comprising an anti-LAG3 antibody and a VEGF antagonist, the individual components may be administered to a subject and/or co-formulated using a variety of dosage combinations. For example, the anti-LAG3 antibody may be administered to a subject and/or contained in a co-formulation in an amount selected from the group consisting of 0.01 mg, 0.02 mg, 0.03 mg, 0.04 mg, 0.05 mg, 0.1 mg, 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg, 3.0 mg, 3.5 mg, 4.0 mg, 4.5 mg, 5.0 mg, 6.0 mg, 7.0 mg, 8.0 mg, 9.0 mg, and 10.0 mg; and the VEGF antagonist (e.g., a VEGF trap such as aflibercept) may be administered to the subject and/or contained in a co-formulation in an amount selected from the group consisting of 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg and 3.0 mg. The combinations/co-formulations may be administered to a subject according to any of the administration regimens disclosed elsewhere herein, including, e.g., twice a week, once every week, once every 2 weeks, once every 3 weeks, once every month, once every 2 months, once every 3 months, once every 4 months, once every 5 months, once every 6 months, etc.

### Administrative Regimens

According to certain embodiments of the present invention, multiple doses of an anti-LAG3 antibody (or a pharmaceutical composition comprising a combination of an anti-LAG3 antibody and any of the additional therapeutically active agents mentioned herein) may be administered to a subject over a defined time course. The methods according to this aspect of the invention comprise sequentially administering to a subject multiple doses of an anti-LAG3 antibody of the invention. As used herein, “sequentially administering” means that each dose of anti-LAG3 antibody is administered to the subject at a different point in time, e.g., on different days separated by a predetermined interval (e.g., hours, days, weeks or months). The present invention includes methods which comprise sequentially administering to the patient a single initial dose of an anti-LAG3 antibody, followed by one or more secondary doses of the anti-LAG3 antibody, and optionally followed by one or more tertiary doses of the anti-LAG3 antibody. The anti-LAG3 antibody may be administered at a dose between 0.1 mg/kg to 100 mg/kg body weight of the subject.

The terms “initial dose,” “secondary doses,” and “tertiary doses,” refer to the temporal sequence of administration of the anti-LAG3 antibody of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-LAG3 antibody, but generally may differ from one another in terms of frequency of administration. In certain embodiments, however, the amount of anti-LAG3 antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain embodiments, the amount of anti-LAG3 antibody contained in the initial, secondary and/or tertiary doses may be sub-optimal or sub-therapeutic. As used herein, the terms “sub-therapeutic” or “sub-optimal” refer to an antibody dose administered at too low a level to produce a therapeutic effect or below the level necessary to treat a disease such as cancer.

In certain exemplary embodiments of the present invention, each secondary and/or tertiary dose is administered 1 to 26 (e.g., 1, 1^{1/2}, 2, 2^{1/2}, 3, 3^{1/2}, 4, 4^{1/2}, 5, 5^{1/2}, 6, 6^{1/2}, 7, 7^{1/2}, 8, 8^{1/2}, 9, 9^{1/2}, 10, 10^{1/2}, 11, 11^{1/2}, 12, 12^{1/2}, 13, 13^{1/2}, 14, 14^{1/2},15, 15^{1/2},16, 16^{1/2},17,17^{1/2}, 18, 18^{1/2},19, 19^{1/2}, 20, 20^{1/2}, 21, 21^{1/2}, 22, 22^{1/2}, 23, 23^{1/2}, 24, 24^{1/2}, 25, 25^{1/2}, 26, 26^{1/2}, or more) weeks after the immediately preceding dose. The phrase “the immediately preceding dose,” as used herein, means, in a sequence of multiple administrations, the dose of anti-LAG3 antibody which is administered to a patient prior to the administration of the very next dose in the sequence with no intervening doses.

The methods according to this aspect of the invention may comprise administering to a patient any number of secondary and/or tertiary doses of an anti-LAG3 antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

### Diagnostic Uses of the Antibodies

The anti-LAG3 antibodies of the present invention may be used to detect and/or measure LAG3 in a sample, e.g., for diagnostic purposes. Some embodiments contemplate the use of one or more antibodies of the present invention in assays to detect a disease or disorder such as cancer, autoimmune disease or viral infection. Exemplary diagnostic assays for LAG3 may comprise, e.g., contacting a sample, obtained from a subject (e.g., a patient), with an anti-LAG3 antibody of the invention, wherein the anti-LAG3 antibody is labeled with a detectable label or reporter molecule or used as a capture ligand to selectively isolate LAG3 from subject samples. Alternatively, an unlabeled anti-LAG3 antibody can be used in diagnostic applications in combination with a secondary antibody which is itself detectably labeled. The detectable label or reporter molecule can be a radioisotope, such as ³H, ¹⁴C, ³²p, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure LAG3 in a sample include enzyme-linked immunosorbent assay (ELISA), radioimmunoassay (RIA), and fluorescence-activated cell sorting (FACS).

Samples that can be used in LAG3 diagnostic assays according to the present invention include any tissue or fluid sample obtainable from a subject, which contains detectable quantities of either LAG3 protein, or fragments thereof, under normal or pathological conditions. Generally, levels of LAG3 in a particular sample obtained from a healthy patient (e.g., a patient not afflicted with cancer or an autoimmune disease) will be measured to initially establish a baseline, or standard, level of LAG3. This baseline level of LAG3 can then be compared against the levels of LAG3 measured in samples obtained from individuals suspected of having a cancer-related condition, or symptoms associated with such condition.

The antibodies specific for LAG3 may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

Aspects of the invention relate to use of the disclosed antibodies as markers for predicting prognosis of cancer or a viral infection in patients. Antibodies of the present invention may be used in diagnostic assays to evaluate prognosis of cancer in a patient and to predict survival.

### EXAMPLES

The following examples are put forth so as to provide those of ordinary skill in the art with a complete disclosure and description of how to make and use the methods and compositions of the invention, and are not intended to limit the scope of what the inventors regard as their invention.

Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Human Antibodies to LAG3

Human antibodies to LAG3 were generated using a fragment of LAG3 that ranges from about amino acids 29-450 of GenBank Accession NP_002277.4 (SEQ ID NO: 582) coupled to a mouse Fc region. The immunogen was administered directly, with an adjuvant to stimulate the immune response, to a VELOCIMMUNE® mouse (i.e., an engineered mouse comprising DNA encoding human Immunoglobulin heavy and kappa light chain variable regions), as described in U.S. Pat. No. 8,502,018 B2, or to a humanized Universal Light Chain (ULC) Veloclmmune® mouse, as described in WO 2013022782. The antibody immune response was monitored by a LAG3-specific immunoassay. When a desired immune response was achieved splenocytes were harvested and fused with mouse myeloma cells to preserve their viability and form hybridoma cell lines. The hybridoma cell lines were screened and selected to identify cell lines that produce LAG3-specific antibodies. Using this technique, and the immunogen described above, several anti-LAG3 chimeric antibodies (i.e., antibodies possessing human variable domains and mouse constant domains) were obtained; exemplary antibodies generated in this manner from the VELOCIMMUNE® mice were designated as H1 M14985N, H1 M14987N, H2M14811 N, H2M14885N, H2M14926N, H2M14927N, H2M14931N, H2M18336N, H2M18337N and H4H14813N.

Anti-LAG3 antibodies were also isolated directly from antigen-positive B cells (from either of the immunized mice) without fusion to myeloma cells, as described in U.S. Pat. No. 7,582,298, herein specifically incorporated by reference in its entirety. Using this method, several fully human anti-LAG3 antibodies (i.e., antibodies possessing human variable domains and human constant domains) were obtained; exemplary antibodies generated in this manner were designated as follows: H4H15477P, H4H15483P, H4H15484P, H4H15491 P, H4H17823P, H4H17826P2, H4H17828P2, H4sH15460P, H4sH15462P, H4sH15463P, H4sH15464P, H4sH15466P, H4sH15467P, H4sH15470P, H4sH15475P, H4sH15479P, H4sH15480P, H4sH15482P, H4sH15488P, H4sH15496P2, H4sH15498P2, H4sH15505P2, H4sH15518P2, H4sH15523P2, H4sH15530P2, H4sH15555P2, H4sH15558P2, H4sH15567P2, and H4H17819P.

Exemplary antibodies H4sH15496P2, H4sH15498P2, H4sH15505P2, H4sH15518P2, H4sH15523P2, H4sH15530P2, H4sH15555P2, H4sH15558P2, and H4sH15567P2 were generated from B-cells from the ULC Velocimmune® mice.

The biological properties of the exemplary antibodies generated in accordance with the methods of this Example are described in detail in the Examples set forth below.

### Example 2: Heavy and Light Chain Variable Region Amino Acid and Nucleotide Sequences

Table 1 sets forth the amino acid sequence identifiers of the heavy and light chain variable regions and CDRs of selected anti-LAG3 antibodies of the invention. The corresponding nucleic acid sequence identifiers are set forth in Table 2.

TABLE 1
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H1M14985N | 2 | 4 | 6 | 8 | 10 | 12 | 14 | 16 |
| | | | | | | | AAS | |
| H1M14987N | 18 | 20 | 22 | 24 | 26 | 28 | 30 | 32 |
| | | | | | | | AAS | |
| H2M14811N | 34 | 36 | 38 | 40 | 42 | 44 | 46 | 48 |
| | | | | | | | GAS | |
| H2M14885N | 50 | 52 | 54 | 56 | 58 | 60 | 62 | 64 |
| | | | | | | | AAS | |
| H2M14926N | 66 | 68 | 70 | 72 | 74 | 76 | 78 | 80 |
| | | | | | | | GAS | |
| H2M14927N | 82 | 84 | 86 | 88 | 90 | 92 | 94 | 96 |
| | | | | | | | ATS | |
| H2M14931N | 98 | 100 | 102 | 104 | 106 | 108 | 110 | 112 |
| | | | | | | | GAS | |
| H2M18336N | 114 | 116 | 118 | 120 | 122 | 124 | 126 | 128 |
| | | | | | | | KVS | |
| H2M18337N | 130 | 132 | 134 | 136 | 138 | 140 | 142 | 144 |
| | | | | | | | GAS | |
| H4H15477P | 146 | 148 | 150 | 152 | 154 | 156 | 158 | 160 |
| | | | | | | | DAS | |
| H4H15483P | 162 | 164 | 166 | 168 | 170 | 172 | 174 | 176 |
| | | | | | | | DAS | |
| H4H15484P | 178 | 180 | 182 | 184 | 186 | 188 | 190 | 192 |
| | | | | | | | DAS | |
| H4H15491P | 194 | 196 | 198 | 200 | 202 | 204 | 206 | 208 |
| | | | | | | | GSS | |
| H4H17823P | 210 | 212 | 214 | 216 | 218 | 220 | 222 | 224 |
| | | | | | | | ATS | |
| H4H17826P2 | 226 | 228 | 230 | 232 | 234 | 236 | 238 | 240 |
| | | | | | | | AAS | |
| H4H17828P2 | 242 | 244 | 246 | 248 | 250 | 252 | 254 | 256 |
| | | | | | | | GAS | |
| H4sH15460P | 258 | 260 | 262 | 264 | 266 | 268 | 270 | 272 |
| | | | | | | | WAS | |
| H4sH15462P | 274 | 276 | 278 | 280 | 282 | 284 | 286 | 288 |
| | | | | | | | WAS | |
| H4sH15463P | 290 | 292 | 294 | 296 | 298 | 300 | 302 | 304 |
| | | | | | | | WAS | |
| H4sH15464P | 306 | 308 | 310 | 312 | 314 | 316 | 318 | 320 |
| | | | | | | | AAS | |
| H4sH15466P | 322 | 324 | 326 | 328 | 330 | 332 | 334 | 336 |
| | | | | | | | LVS | |
| H4sH15467P | 338 | 340 | 342 | 344 | 346 | 348 | 350 | 352 |
| | | | | | | | DAS | |
| H4sH15470P | 354 | 356 | 358 | 360 | 362 | 364 | 366 | 368 |
| | | | | | | | AAS | |
| H4sH15475P | 370 | 372 | 374 | 376 | 378 | 380 | 382 | 384 |
| | | | | | | | KIS | |
| H4sH15479P | 386 | 388 | 390 | 392 | 394 | 396 | 398 | 400 |
| | | | | | | | DAS | |
| H4sH15480P | 402 | 404 | 406 | 408 | 410 | 412 | 414 | 416 |
| | | | | | | | GAS | |
| H4sH15482P | 418 | 420 | 422 | 424 | 426 | 428 | 430 | 432 |
| | | | | | | | DAS | |
| H4sH15488P | 434 | 436 | 438 | 440 | 442 | 444 | 446 | 448 |
| | | | | | | | LVS | |
| H4sH15496P2 | 450 | 452 | 454 | 456 | 522 | 524 | 526 | 528 |
| | | | | | | | AAS | |
| H4sH15498P2 | 458 | 460 | 462 | 464 | 522 | 524 | 526 | 528 |
| | | | | | | | AAS | |
| H4sH15505P2 | 466 | 468 | 470 | 472 | 522 | 524 | 526 | 528 |
| | | | | | | | AAS | |
| H4sH15518P2 | 474 | 476 | 478 | 480 | 522 | 524 | 526 | 528 |
| | | | | | | | AAS | |
| H4sH15523P2 | 482 | 484 | 486 | 488 | 522 | 524 | 526 | 528 |
| | | | | | | | AAS | |
| H4sH15530P2 | 490 | 492 | 494 | 496 | 522 | 524 | 526 | 528 |
| | | | | | | | AAS | |
| H4sH15555P2 | 498 | 500 | 502 | 504 | 530 | 532 | 534 | 536 |
| | | | | | | | GAS | |
| H4sH15558P2 | 506 | 508 | 510 | 512 | 530 | 532 | 534 | 536 |
| | | | | | | | GAS | |
| H4sH15567P2 | 514 | 516 | 518 | 520 | 530 | 532 | 534 | 536 |
| | | | | | | | GAS | |
| H4H14813N | 538 | 540 | 542 | 544 | 546 | 548 | 550 | 552 |
| | | | | | | | GAS | |
| H4H17819P | 554 | 556 | 558 | 560 | 562 | 564 | 566 | 568 |
| | | | | | | | AAS | |

TABLE 2
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H1M14985N | 1 | 3 | 5 | 7 | 9 | 11 | 13 | 15 |
| | | | | | | | gctgcatcc | |
| H1M14987N | 17 | 19 | 21 | 23 | 25 | 27 | 29 | 31 |
| | | | | | | | gctgcatcc | |
| H2M14811N | 33 | 35 | 37 | 39 | 41 | 43 | 45 | 47 |
| | | | | | | | ggtgcatcc | |
| H2M14885N | 49 | 51 | 53 | 55 | 57 | 59 | 61 | 63 |
| | | | | | | | gctgcatcc | |
| H2M14926N | 65 | 67 | 69 | 71 | 73 | 75 | 77 | 79 |
| | | | | | | | ggtgcatcc | |
| H2M14927N | 81 | 83 | 85 | 87 | 89 | 91 | 93 | 95 |
| | | | | | | | gctacatcc | |
| H2M14931N | 97 | 99 | 101 | 103 | 105 | 107 | 109 | 111 |
| | | | | | | | ggtgcgtcc | |
| H2M18336N | 113 | 115 | 117 | 119 | 121 | 123 | 125 | 127 |
| | | | | | | | aaggtttct | |
| H2M18337N | 129 | 131 | 133 | 135 | 137 | 139 | 141 | 143 |
| | | | | | | | ggtgcttcc | |
| H4H15477P | 145 | 147 | 149 | 151 | 153 | 155 | 157 | 159 |
| | | | | | | | gatgcatcc | |
| H4H15483P | 161 | 163 | 165 | 167 | 169 | 171 | 173 | 175 |
| | | | | | | | gatgcatcc | |
| H4H15484P | 177 | 179 | 181 | 183 | 185 | 187 | 189 | 191 |
| | | | | | | | gatgcatcc | |
| H4H15491P | 193 | 195 | 197 | 199 | 201 | 203 | 205 | 207 |
| | | | | | | | ggttcatcc | |
| H4H17823P | 209 | 211 | 213 | 215 | 217 | 219 | 221 | 223 |
| | | | | | | | gctacatcc | |
| H4H17826P2 | 225 | 227 | 229 | 231 | 233 | 235 | 237 | 239 |
| | | | | | | | gctgcatcc | |
| H4H17828P2 | 241 | 243 | 245 | 247 | 249 | 251 | 253 | 255 |
| | | | | | | | ggtgcatcc | |
| H4sH15460P | 257 | 259 | 261 | 263 | 265 | 267 | 269 | 271 |
| | | | | | | | tgggcatct | |
| H4sH15462P | 273 | 275 | 277 | 279 | 281 | 283 | 285 | 287 |
| | | | | | | | tgggcatct | |
| H4sH15463P | 289 | 291 | 293 | 295 | 297 | 299 | 301 | 303 |
| | | | | | | | tgggcatct | |
| H4sH15464P | 305 | 307 | 309 | 311 | 313 | 315 | 317 | 319 |
| | | | | | | | gctgcatcc | |
| H4sH15466P | 321 | 323 | 325 | 327 | 329 | 331 | 333 | 335 |
| | | | | | | | ttggtttct | |
| H4sH15467P | 337 | 339 | 341 | 343 | 345 | 347 | 349 | 351 |
| | | | | | | | gatgcatcc | |
| H4sH15470P | 353 | 355 | 357 | 359 | 361 | 363 | 365 | 367 |
| | | | | | | | gctgcatca | |
| H4sH15475P | 369 | 371 | 373 | 375 | 377 | 379 | 381 | 383 |
| | | | | | | | aagatttct | |
| H4sH15479P | 385 | 387 | 389 | 391 | 393 | 395 | 397 | 399 |
| | | | | | | | gatgcatcc | |
| H4sH15480P | 401 | 403 | 405 | 407 | 409 | 411 | 413 | 415 |
| | | | | | | | ggtgcatcc | |
| H4sH15482P | 417 | 419 | 421 | 423 | 425 | 427 | 429 | 431 |
| | | | | | | | gatgcatcc | |
| H4sH15488P | 433 | 435 | 437 | 439 | 441 | 443 | 445 | 447 |
| | | | | | | | ttggtttct | |
| H4sH15496P2 | 449 | 451 | 453 | 455 | 521 | 523 | 525 | 527 |
| | | | | | | | gctgcatcc | |
| H4sH15498P2 | 457 | 459 | 461 | 463 | 521 | 523 | 525 | 527 |
| | | | | | | | gctgcatcc | |
| H4sH15505P2 | 465 | 467 | 469 | 471 | 521 | 523 | 525 | 527 |
| | | | | | | | gctgcatcc | |
| H4sH15518P2 | 473 | 475 | 477 | 479 | 521 | 523 | 525 | 527 |
| | | | | | | | gctgcatcc | |
| H4sH15523P2 | 481 | 483 | 485 | 487 | 521 | 523 | 525 | 527 |
| | | | | | | | gctgcatcc | |
| H4sH15530P2 | 489 | 491 | 493 | 495 | 521 | 523 | 525 | 527 |
| | | | | | | | gctgcatcc | |
| H4sH15555P2 | 497 | 499 | 501 | 503 | 529 | 531 | 533 | 535 |
| | | | | | | | ggtgcatcc | |
| H4sH15558P2 | 505 | 507 | 509 | 511 | 529 | 531 | 533 | 535 |
| | | | | | | | ggtgcatcc | |
| H4sH15567P2 | 513 | 515 | 517 | 519 | 529 | 531 | 533 | 535 |
| | | | | | | | ggtgcatcc | |
| H4H14813N | 537 | 539 | 541 | 543 | 545 | 547 | 549 | 551 |
| | | | | | | | ggtgcatcc | |
| H4H17819P | 553 | 555 | 557 | 559 | 561 | 563 | 565 | 567 |
| | | | | | | | gctgcatcc | |

Antibodies are typically referred to herein according to the following nomenclature: Fc prefix (e.g. “H1M,” “H4sH,” “H4H,” etc.), followed by a numerical identifier (e.g. “14813,‘ ’17828,” etc., as shown in Table 1), followed by a “P,” “P2,” or ‘N’ suffix. Thus, according to this nomenclature, an antibody may be referred to herein as, e.g., “H4sH14813N,” “H4H17819P,” “H4H17828P2,” etc. The H4sH and H4H prefixes on the antibody designations used herein indicate the particular Fc region isotype of the antibody. For example, an “H4sH” antibody has a human IgG4 Fc with 2 or more amino acid changes as disclosed in US20140243504 (herein incorporated in its entirety), an “H4H” antibody has a human IgG4 Fc with a serine to proline mutation in the hinge region (S1 08P), an “H1 M” antibody has a mouse IgG1 Fc, and an “H2M” antibody has a mouse IgG2 Fc (all variable regions are fully human as denoted by the first ‘H’ in the antibody designation). As will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs) -which are indicated by the numerical identifiers shown in Table 1-will remain the same, and the binding properties to antigen are expected to be identical or substantially similar regardless of the nature of the Fc domain.

In certain embodiments, selected antibodies with a mouse IgG1 Fc were converted to antibodies with human IgG4 Fc. In certain embodiments, the antibody comprises a human IgG4 Fc with 2 or more amino acid changes as disclosed in US20100331527 (herein incorporated in its entirety). In one embodiment, the IgG4 Fc domain comprises a serine to proline mutation in the hinge region (S1 08P) to promote dimer stabilization. In certain embodiments, the Fc region of the antibodies of the present invention comprises amino acid sequences of SEQ ID NOs: 569, 570, 571, 572 or 573. Table 3 sets forth the amino acid sequence identifiers of heavy chain and light chain sequences of selected anti-LAG3 antibodies with human IgG4 Fc.

TABLE 3
Heavy Chain and Light Chain Amino Acid Sequence Identifiers

| Antibody | SEQ ID NOs: | |
|---|---|---|
| Designation | Heavy Chain | Light Chain |
| H4sH15482P | 577 | 578 |
| H4H15482P | 579 | 578 |
| H4sH14813N | 580 | 581 |

Each heavy chain sequence in Table 3 comprised a variable region (V_{H} or HCVR; comprising HCDR1, HCDR2 and HCDR3) and a constant region (comprising C_{H}1, C_{H}2 and C_{H}3 domains). Each light chain sequence in Table 3 comprised a variable region (V_{L} or LCVR; comprising LCDR1, LCDR2 and LCDR3) and a constant region (C_{L}). SEQ ID NO: 577 comprised a HCVR comprising amino acids 1-123 and a constant region comprising amino acids 124-449. SEQ ID NO: 578 comprised a LCVR comprising amino acids 1-107 and a constant region comprising amino acids 108-214. SEQ ID NO: 579 comprised a HCVR comprising amino acids 1-123 and a constant region comprising amino acids 124-450. SEQ ID NO: 580 comprised a HCVR comprising amino acids 1-119 and a constant region comprising amino acids 120-445. SEQ ID NO: 581 comprised a LCVR comprising amino acids 1-107 and a constant region comprising amino acids 108-214.

### Control Constructs Used in the Following Examples

Two control constructs (anti-LAG3 antibodies) were included in the following experiments for comparative purposes: “Comparator 1,” a human monoclonal antibody against LAG3 having V_{H}/V_{L} sequences of antibody “25F7” according to WO2010/019570; and “Comparator 2,” a human monoclonal antibody against LAG3 having V_{H}/V_{L} sequences of antibody “v3.5” according to US2014/0093511.

### Example 3: Antibody binding to LAG3 as determined by Surface Plasmon Resonance

Binding association and dissociation rate constants (kₐ and k_{d}, respectively), equilibrium dissociation constants and dissociation half-lives (K_{D} and t_{1/2}, respectively) for antigens binding to purified anti-LAG3 antibodies were determined using a real-time surface plasmon resonance biosensor assay on a BiaCore 4000 or BiaCore T200 instrument. The BiaCore sensor surface was derivatized with either a polyclonal rabbit anti-mouse antibody (GE, #BR-1008-38) or with a monoclonal mouse anti-human Fc antibody (GE, #BR-1008-39) to capture approximately 50-85 RUs of anti-LAG3 monoclonal antibodies, expressed with either a mouse Fc or a human Fc, respectively. The LAG3 reagents tested for binding to the anti-LAG3 antibodies included recombinant human LAG3 expressed with a C-terminal myc-myc-hexahistidine tag (hLAG3-mmh; SEQ ID: 574) and recombinant human LAG3 expressed with a C-terminal mouse IgG2a mFc tag (hLAG3-mFc; SEQ ID: 575). Different concentrations (50, 25, 12.5 and 6.25 nM) of LAG3 reagents were injected over the anti-LAG3 monoclonal antibody captured surface at a flow rate of 50 μL/min on the BiaCore T200. The binding of the LAG3 reagents to captured monoclonal antibodies was monitored for 4 minutes while their dissociation from the antibodies was monitored for 8 minutes in HBST running buffer (0.01 M HEPES pH 7.4, 0.15 M NaCl, 3 mM EDTA, 0.05% v/v Surfactant P20). Experiments were performed at 25° C. and 37° C. Kinetic association (kₐ) and dissociation (k_{d}) rate constants were determined by processing and fitting the data to a 1:1 binding model using Scrubber 2.0c curve fitting software. Binding dissociation equilibrium constants (K_{D}) and dissociative half-lives (t_{1/2}) were then calculated from the kinetic rate constants as: K_{D} (M)=k_{d}/kₐ and t_{1/2}. (min)=[ln2/(60*k_{d})].

Binding kinetics parameters for different anti-LAG3 monoclonal antibodies binding to monomeric (tagged with mmH) or dimeric (tagged with mFc) LAG3 reagents at 25° C. and 37° C. are tabulated in Tables 4-7.

TABLE 4
Binding Kinetics parameters of anti-LAG3 monoclonal
antibodies binding to human LAG3-mmh at 25° C.

| | 25° C. | | | |
|---|---|---|---|---|
| | kₐ | k_{d} | K_{D} | t_{1/2} |
| Ab ID | (I/mol*s) | (1/s) | [M] | (min) |
| H4H15479P | 1.09E+06 | 5.35E−05 | 4.93E−11 | 215.9 |
| H4sH15479P | 1.23E+06 | 5.98E−05 | 4.87E−11 | 193.1 |
| H4sH14813N | 4.45E+05 | 7.89E−05 | 1.77E−10 | 146.4 |
| H4H14813N | 4.11E+05 | 8.80E−05 | 2.14E−10 | 131.2 |
| H4H15498P2 | 3.01E+05 | 7.81E−05 | 2.60E−10 | 147.9 |
| H4H17819P | 1.13E+06 | 4.43E−04 | 3.91E−10 | 26 |
| H4H17823P | 1.04E+06 | 4.47E−04 | 4.29E−10 | 25.8 |
| H4sH15498P2 | 1.57E+05 | 1.01E−04 | 6.45E−10 | 114 |
| H4H15482P | 1.99E+05 | 1.94E−04 | 9.72E−10 | 59.7 |
| H4sH15482P | 2.12E+05 | 2.60E−04 | 1.23E−09 | 44.5 |
| H4H15483P | 1.88E+05 | 3.70E−04 | 1.97E−09 | 31.2 |
| H4H17828P2 | 6.12E+05 | 1.41E−03 | 2.31E−09 | 8.2 |
| H4H15462P | 9.39E+05 | 5.40E−03 | 5.75E−09 | 2.1 |
| H4sH15462P | 1.07E+06 | 6.97E−03 | 6.51E−09 | 1.7 |
| H4H17826P2 | 4.50E+05 | 4.57E−03 | 1.02E−08 | 2.5 |
| Isotype control 1 | NB | NB | NB | NB |
| Isotype control 2 | NB | NB | NB | NB |
| Comparator 1 | 1.09E+06 | 8.70E−04 | 8.02E−10 | 13.3 |
| Comparator 2 | 1.01E+06 | 5.82E−04 | 5.77E−10 | 19.9 |

*NB indicates that under experimental conditions, LAG3 reagent did not bind to the captured anti-LAG3 monoclonal antibody

Table 4 shows that at 25° C., all the anti-LAG3 antibodies of the invention bound to hLAG3-mmh with K_{D} values ranging from 49 pM to 10 nM, while the comparators bound with K_{D} values of 0.58 nM and 0.8 nM.

TABLE 5
Binding kinetics parameters of anti-LAG3 monoclonal
antibodies binding to hLAG3-mmh at 37° C.

| | 37° C. | | | |
|---|---|---|---|---|
| | kₐ | k_{d} | K_{D}* | t_{1/2} |
| Ab ID | (I/mol*s) | (1/s) | [M] | (min) |
| H4H15479P | 1.27E+06 | 4.08E−05 | 3.21E−11 | 283 |
| H4sH15479P | 1.24E+06 | 4.24E−05 | 3.41E−11 | 272.5 |
| H4H15498P2 | 3.81E+05 | 2.96E−05 | 7.83E−11 | 390.2 |
| H4sH15498P2 | 3.78E+05 | 7.08E−05 | 1.85E−10 | 163.1 |
| H4sH14813N | 5.78E+05 | 1.54E−04 | 2.66E−10 | 75.1 |
| H4H14813N | 5.35E+05 | 1.52E−04 | 2.84E−10 | 76 |
| H4H17823P | 1.76E+06 | 9.24E−04 | 5.24E−10 | 12.5 |
| H4H17819P | 1.59E+06 | 8.76E−04 | 5.52E−10 | 13.2 |
| H4H15483P | 7.74E+05 | 1.11E−03 | 1.43E−09 | 10.4 |
| H4sH15482P | 2.81E+05 | 5.12E−04 | 1.82E−09 | 22.6 |
| H4H17828P2 | 1.10E+06 | 2.07E−03 | 1.89E−09 | 5.6 |
| H4H15482P | 2.92E+05 | 5.70E−04 | 1.96E−09 | 20.2 |
| H4H17826P2 | 1.07E+06 | 2.14E−03 | 1.99E−09 | 5.4 |
| H4H15462P | 1.08E+06 | 6.26E−03 | 5.80E−09 | 1.8 |
| H4sH15462P | 1.01E+06 | 7.72E−03 | 7.66E−09 | 1.5 |
| Isotype control 1 | NB | NB | NB | NB |
| Isotype control 2 | NB | NB | NB | NB |
| Comparator 1 | 1.23E+06 | 3.74E−03 | 3.03E−09 | 3.1 |
| Comparator 2 | 1.10E+06 | 2.31E−03 | 2.10E−09 | 5 |

*NB indicates that under the experimental conditions, LAG3 reagent did not bind to the captured anti-LAG3 monoclonal antibody.

Table 5 shows that at 37° C., all the anti-LAG3 antibodies of the invention bound to hLAG3-mmh with K_{D} values ranging from 32pM to 7.66 nM, while the comparators bound with K_{D} values ranging of 2.1 nM and 3.0 nM.

Data in Tables 4 and 5 indicate that all anti-LAG3 antibodies bind to monomeric hLAG3-mmh with very similar affinities at both 253 and 371C.

TABLE 6
Binding Kinetics parameters of anti-LAG3 monoclonal antibodies
binding to hLAG3 dimer (hLAG3-mFc) at 25° C.

| | 25° C. | | | |
|---|---|---|---|---|
| | kₐ | k_{d} | K_{D} | t_{1/2} |
| Ab ID | (I/mol*s) | (1/s) | [M] | (min) |
| H4H15479P | 2.30E+06 | 1.00E−05 | 4.35E−12 | ≥1155.0 |
| H4sH15479P | 3.05E+06 | 1.00E−05 | 3.28E−12 | ≥1155.0 |
| H4H15483P | 1.34E+06 | 1.00E−05 | 7.45E−12 | ≥1155.0 |
| H4H14813N | 9.58E+05 | 1.00E−05 | 1.04E−11 | ≥1155.0 |
| H4sH14813N | 1.80E+06 | 1.90E−05 | 1.06E−11 | 606.6 |
| H4H15482P | 7.58E+05 | 1.00E−05 | 1.32E−11 | ≥1155.0 |
| H4sH15482P | 7.09E+05 | 1.00E−05 | 1.41E−11 | ≥1155.0 |
| H4H15498P2 | 3.63E+05 | 1.00E−05 | 2.76E−11 | ≥1155.0 |
| H4sH15498P2 | 2.84E+05 | 1.00E−05 | 3.52E−11 | ≥1155.0 |
| H4H17819P | 1.45E+06 | 1.80E−04 | 1.24E−10 | 64.2 |
| H4H17823P | 1.26E+06 | 2.18E−04 | 1.74E−10 | 52.9 |
| H4H17828P2 | 1.88E+06 | 4.60E−04 | 2.45E−10 | 25.1 |
| H4sH15462P | 2.34E+06 | 1.90E−03 | 8.14E−10 | 6.1 |
| H4H17826P2 | 6.91E+05 | 5.64E−04 | 8.17E−10 | 20.5 |
| H4H15462P | 2.02E+06 | 2.03E−03 | 1.01E−09 | 5.7 |
| Isotype control 1 | NB* | NB | NB | NB |
| Isotype control 2 | NB | NB | NB | NB |
| Comparator 1 | 2.69E+06 | 4.50E−04 | 1.67E−10 | 25.7 |
| Comparator 2 | 3.09E+06 | 3.00E−04 | 9.68E−11 | 38.6 |

*NB indicates that under the experimental conditions, LAG3 reagent did not bind to the captured anti-LAG3 monoclonal antibody.

Table 6 shows that at 25° C., all the anti-LAG3 antibodies of the invention bound to hLAG3 dimer proteins with K_{D} values ranging from 4.3pM-1 0.0 nM, while the comparators bound with K_{D} values of 97 pM and 0.16 nM.

TABLE 7
Binding Kinetics parameters of anti-LAG3 monoclonal antibodies
binding to hLAG3 dimer (hLAG3-mFc) at 37° C.

| | 37° C. | | | |
|---|---|---|---|---|
| | kₐ | k_{d} | K_{D} | t_{1/2} |
| Ab ID | (I/mol*s) | (1/s) | [M] | (min) |
| H4H15479P | 2.50E+06 | 1.00E−05 | 3.99E−12 | ≥1155.0 |
| H4sH15479P | 2.92E+06 | 1.00E−05 | 3.42E−12 | ≥1155.0 |
| H4sH15498P2 | 5.15E+05 | 1.00E−05 | 1.94E−11 | ≥1155.0 |
| H4H15498P2 | 4.60E+05 | 1.00E−05 | 2.17E−11 | ≥1155.0 |
| H4H14813N | 4.27E+06 | 1.17E−04 | 2.74E−11 | 98.8 |
| H4sH14813N | 3.42E+06 | 1.01E−04 | 2.95E−11 | 114.4 |
| H4sH15482P | 5.19E+06 | 1.64E−04 | 3.16E−11 | 70.3 |
| H4H15482P | 1.01E+06 | 9.01E−05 | 8.91E−11 | 128.1 |
| H4H15483P | 1.38E+06 | 2.98E−04 | 2.15E−10 | 38.8 |
| H4H17826P2 | 1.98E+06 | 5.47E−04 | 2.77E−10 | 21.1 |
| H4H17819P | 1.66E+06 | 5.27E−04 | 3.17E−10 | 21.9 |
| H4H17828P2 | 1.73E+06 | 7.18E−04 | 4.15E−10 | 16.1 |
| H4H15462P | 2.16E+06 | 8.95E−04 | 4.15E−10 | 12.9 |
| H4H17823P | 1.34E+06 | 5.85E−04 | 4.37E−10 | 19.7 |
| H4sH15462P | 1.80E+06 | 1.57E−03 | 8.76E−10 | 7.3 |
| Isotype control 1 | NB | NB | NB | NB |
| Isotype control 2 | NB | NB | NB | NB |
| Comparator 1 | 3.44E+06 | 1.56E−03 | 4.54E−10 | 7.4 |
| Comparator 2 | 4.36E+06 | 1.13E−03 | 2.58E−10 | 10.3 |

*NB indicates that under the experimental conditions, LAG3 reagent did not bind to the captured anti-LAG3 monoclonal antibody

Table 7 shows that at 37° C., all the anti-LAG3 antibodies of the invention bound to hLAG3 dimer proteins with K_{D} values ranging from 4.0 pM-0.9 nM, while the comparators bound with K_{D} values of 0.26 nM and 0.45 nM.

Data in Tables 6 and 7 indicate that all anti-LAG3 antibodies bind to hLAG3 dimer reagents with similar affinities at both 25° and 37° C.

### Example 4: Octet cross-competition between anti-LAG3 antibodies

To assess whether two antibodies compete with one another for binding to monomeric human LAG3, (hLAG3.mmh) binding competition between anti-LAG3 monoclonal antibodies was determined using a real time, label-free bio-layer interferometry assay on an Octet RED384 biosensor (Pall ForteBio Corp.). Cross competition experiment were run at 25° C. in 0.01 M HEPES pH7.4, 0.15M NaCl, 0.05% v/v Surfactant Tween-20, 0.1 mg/mL BSA (Octet HBS-P buffer) with the plate shaking at the speed of 1000 rpm. All anti-LAG3 antibody and hLAG3 solutions tested were formulated in Octet HBS-P buffer. To assess whether 2 antibodies were able to compete with one another for binding to hLAG3, approximately ˜0.6-0.85 nm of hLAG3.mmh was first captured on anti-His coated Octet biosensor tips from wells containing 5 μg/mL of hLAG3 for 75 seconds. The hLAG3 captured Octet biosensor tips were saturated by submerging for 5 minutes into wells containing 50 ug/ml of the first anti-LAG3 monoclonal antibody (hereby referred as mAb-1), followed by submerging in wells containing the second anti-LAG3 monoclonal antibody (hereby referred as mAb-2). Between each steps the Octet biosensor tips were washed in HBS-P buffer for 30 seconds.

The real-time binding response was monitored during the course of the experiment and the binding response at the end of every step was recorded. The response of hLAG3 binding to mAb-1 and then to the blocking mAb was corrected for background binding, compared and competitive/non-competitive behavior of different anti-Lag monoclonal antibodies was determined.

Table 8 explicitly defines the relationships of antibodies competing in both directions, independent of the order of binding.

TABLE 8
Cross-competition of anti-LAG3 antibodies
for binding to monomeric hLAG3

| First mAb | |
|---|---|
| (mAb-1) | |
| Captured using | |
| AHC Octet | mAb-2 Antibodies Shown to Compete with |
| Biosensors | mAb-1* |
| H4sH15482P | H4sH15479P, H4sH14813N, H4H14813N, |
| | H4H15479P, H4H15482P, H4H15483P |
| H4sH15479P | H4sH15482P, H4sH14813N, H4H14813N, |
| | H4H15479P, H4H15482P, H4H15483P |
| H4sH14813N | H4sH15482P, H4sH15479P, H4H14813N, |
| | H4H15479P, H4H15482P, H4H15483P |
| H4H14813N | H4sH15482P, H4sH15479P, H4sH14813N, |
| | H4H15479P, H4H15482P, H4H15483P |
| H4H15479P | H4sH15482P, H4sH15479P, H4sH14813N, |
| | H4H14813N, H4H15482P, H4H15483P |
| H4H15482P | H4sH15482P, H4sH15479P, H4sH14813N, |
| | H4H14813N, H4H15479P, H4H15483P |
| H4H15483P | H4sH15482P, H4sH15479P, H4sH14813N, |
| | H4H14813N, H4H15479P, H4H15482P |
| H4sH15498P2 | H4H15498P2, H4H17828P2, H4H17819P, |
| | H4H17823P |
| H4H15498P2 | H4sH15498P2, H4H17828P2, H4H17819P, |
| | H4H17823P |
| H4H17828P2 | H4sH15498P2, H4H15498P2, H4H17819P, |
| | H4H17823P |
| H4H17819P | H4sH15498P2, H4H15498P2, H4H17828P2, |
| | H4H17823P |
| H4H17823P | H4sH15498P2, H4H15498P2, H4H17828P2, |
| | H4H17819P |
| H4sH15462P | none |
| H4H15462P | none |
| H4H17826P2 | none |

(*Self-competing mAb2s are not listed)

### Example 5: Antibody binding to cells overexpressing LAG3 as determined by flow cytometry

HEK293 cells (HEK293; ATCC, #CRL-1573) stable cell lines that express recombinant hLAG3 (NCBI Accession No. NP_002277.4) or monkey LAG3 (mfLAG3) [cynomolgus monkey sequence NCBI Accession No. XP_005570011.1 was further modified to replace the “X” at amino acid position 74 with proline based on Rhesus macaque (Macaca mulata) sequence NCBI Accession No. XP_001108923.1] (SEQ ID NO: 576) at the cell surface were developed and used to determine the binding specificity of anti-LAG3 monoclonal antibodies of the invention by flow cytometry analysis.

Binding of anti-LAG3 antibodies was assessed as follows: Stable HEK293 cells expressing either hLAG3 or mfLAG3 were washed 1× with D-PBS (Irvine Scientific Cat #9240), trypsinized (Specialty Media Cat #SM-2004-C) and blocked with HEK293 cell culture medium (DME+10% FBS+P/S/G+ non-essential amino acids). After centrifugation cells were resuspended at 2.0×106 cells/mL in staining buffer (D-PBS+2% FBS). The anti-LAG3 antibodies and isotype controls were serially diluted in staining buffer with a dose ranging from 5 pM to 100 nM including a no antibody buffer only control. The serially diluted antibodies were added to the cell suspension and incubated for 15-30 minutes on ice. The cells were centrifuged and pellets were washed with staining buffer to remove unbound antibodies. Subsequently, cells were incubated for 15-30 minutes on ice with an allophycocyanin-conjugated secondary F(ab′)2 recognizing human Fc (Jackson ImmunoResearch, #109-136-170). The cells were centrifuged and pellets were washed with staining buffer to remove unbound secondary F(ab′)2 and fixed overnight with a 1:1 dilution of Cytofix (BD Biosciences, #554655) and staining buffer. Following day, fixed cells were centrifuged and pellets were washed with staining buffer, resuspended and filtered. Fluorescence measurements were acquired on HyperCyt® cytometer and analyzed in ForeCyt™ (IntelliCyt; Albuquerque, NM) to determine the mean fluorescence intensities (MFI). The EC₅Ovalues were calculated from a four-parameter logistic equation over an 11-point response curve using GraphPad Prism. EC₅₀ was defined as the concentration of antibody at which 50% of maximal binding signal is detected. The MFI ratio for each antibody was calculated by dividing the MFI at 100 nM by MFI at 0 nM of antibody concentration.

TABLE 9
Binding of anti-LAG3 antibodies to HEK293
wild-type (wt) cells as determined by FACS

| | FACS on HEK293 wt | |
|---|---|---|
| | | Ratio of MFI |
| Ab PID# | EC50 [M] | (100 nM/0 nM) |
| hlgG4 isotype control | — | 1.5 |
| H4sH15462P | — | 0.9 |
| H4H15462P | — | 8.2 |
| H4sH15479P | — | 1.1 |
| H4H15479P | — | 1.7 |
| H4sH15482 | — | 1.2 |
| H4H15482P | — | 4.9 |
| H4sH15498P2 | — | 1 |
| H4H15498P2 | — | 1 |
| H4sH14813N | — | 1.5 |
| H4H14813N | — | 1.3 |
| H4H17828P2 | — | 1.1 |
| H4H17826P2 | — | 2.5 |
| H4H17823P | — | 2.5 |
| H4H15483P | — | 2.6 |
| H4H17819P | — | 1.1 |
| Comparator 1 | — | 1 |
| Comparator 2 | — | 1.6 |

As shown in Table 9, comparators and isotype controls did not demonstrate any measurable binding to parental wild type HEK293 cells by FACS analysis. The calculated MFI ratios ranged between 1 0.0 and 1 0.6×. A few anti-LAG3 antibodies showed higher ratios on wild type cells, suggesting a nonspecific binding, with the calculated MFI ratios ranging between 0.9 and 8.2×. Under these staining conditions no EC₅Ovalues were determined for the anti-LAG3 antibodies as well as the comparators.

TABLE 10
Binding of anti-LAG3 antibodies to HEK293/hLag3
cells as determined by FACS

| | FACS on HEK293/hLAG3 | |
|---|---|---|
| | | Ratio of MFI |
| Ab PID# | EC50 [M] | (100 nM/0 nM) |
| hIgG4 isotype control | — | 1.8 |
| H4sH15462P | 6.65E−10 | 181.6 |
| H4H15462P | 3E−10 | 62.6 |
| H4sH15479P | 9.03E−10 | 198.7 |
| H4H15479P | 7.52E−10 | 203.2 |
| H4sH15482 | 1.11E−09 | 140.8 |
| H4H15482P | 1.91E−09 | 103.7 |
| H4sH15498P2 | 7.92E−09 | 400.6 |
| H4H15498P2 | 5.76E−09 | 305 |
| H4sH14813N | 1.18E−09 | 197.2 |
| H4H14813N | 1.44E−09 | 208.5 |
| H4H17828P2 | 2.65E−09 | 355.2 |
| H4H17826P2 | 3.86E−09 | 414.2 |
| H4H17823P | 1.25E−09 | 304.9 |
| H4H15483P | 2.31E−09 | 206.3 |
| H4H17819P | 2.79E−09 | 435.8 |
| Comparator 1 | 7.52E−10 | 188.2 |
| Comparator 2 | 6.5E−10 | 150.9 |

As shown in Table 10, isotype controls did not demonstrate any measurable binding to HEK293/hLag3 cells. All 15 anti-LAG3 antibodies of the invention showed a significant binding to HEK293/hLag3 cells with EC₅Ovalues ranging from 300pM to 7.9 nM. The calculated MFI ratios ranged from 62-414×. The EC₅Ovalues for comparators were 0.65 nM and 0.75 nM in the binding assay and MFI ratios were less than 200× for both the antibodies.

TABLE 11
Binding of anti-LAG3 antibodies to HEK293/mfLag3
cells as determined by FACS

| | FACS on HEK293/mfLAG3 | |
|---|---|---|
| | | Ratio of MFI |
| Ab PID# | EC50 [M] | (100 nM/0 nM) |
| hlgG4 isotype control | — | 1.3 |
| H4sH15462P | — | 1 |
| H4H15462P | — | 2.9 |
| H4sH15479P | 9.02E−10 | 20.7 |
| H4H15479P | 1.35E−09 | 21.5 |
| H4sH15482 | 1.15E−09 | 10 |
| H4H15482P | 2.2E−09 | 11.9 |
| H4sH15498P2 | >10 nM | 12.1 |
| H4H15498P2 | >10 nM | 12.8 |
| H4sH14813N | 1.57E−09 | 16.7 |
| H4H14813N | 1.42E−09 | 17.1 |
| H4H17826P2 | >10 nM | 14.3 |
| H4H17823P | >10 nM | 12.4 |
| H4H15483P | >10 nM | 8.8 |
| H4H17819P | >10 nM | 12.1 |
| Comparator 1 | >10 nM | 6.7 |
| Comparator 2 | >10 nM | 8.6 |

As shown in Table 11, isotype controls did not demonstrate any measurable binding to HEK293/mfLag3 cells. A total of 13 out of 15 anti-LAG3 antibodies of the invention showed a significant binding to HEK293/mfLAG3 cells with EC₅Ovalues ranging from 900pM to greater than nM. The calculated MFI ratios ranged from 8.8× to 21.5×. The EC₅Ovalues for the comparators were greater than 10 nM and calculated MFI ratios were 6.7× and 8.6×.

### Example 6: Antibody Binding to Cells Overexpressing LAG3 as Determined by Electrochemiluminescence Immunoassay

To investigate the ability of a panel of anti-LAG3 monoclonal antibodies to bind cell-surface expressed LAG3, an in vitro binding assay utilizing human and monkey LAG3 expressing cell lines in an electrochemiluminescence based detection platform [Meso Scale Diagnostics, Rockville, MD —(MSD)] was developed.

The HEK293 (ATCC, #CRL-1573) stable cell lines were generated by lentivirus transduction to express recombinant hLAG3 (NCBI Accession No. NP_002277.4) or monkey LAG3 (mfLAG3) [cynomolgus monkey sequence Macaca fascicularis Accession No.

XP_005570011.1, modified to replace the “X” at amino acid position 74 with proline based on Rhesus macaque (Macaca mulata) sequence NCBI Accession No. XP_001108923.1) (SEQ ID NO: 576). The parental HEK293 cell line, which has no detectable expression of LAG3 by fluorescence activated cell sorting (FACS), was included in the experiment as a background binding control. Unrelated human IgG4 and hlgG4s were included as isotype control antibodies.

Experiments were carried out according to following procedure. Cells from the three cell lines described above were rinsed once in 1×PBS buffer without Ca²⁺/Mg²⁺ followed by 10-minute incubation at 37° C. with Enzyme Free Cell Dissociation Solution. The detached cells were washed one time with 1×PBS with Ca²⁺/Mg²⁺+ and counted with a Cellometer™ Auto T4 cell counter (Nexcelom Bioscience). Approximately 10,000 cells per well in the cell-washing buffer were seeded into the 96-well carbon electrode plates (MULTI-ARRAY high bind plate, MSD) and incubated for 1 hour at 37° C. to allow the cells to adhere. Nonspecific binding sites were blocked by 2% BSA (w/v) in PBS for 1 hour at room temperature. To the plate-bound cells, solutions of anti-LAG3 antibodies in serial dilutions ranging from 1.7pM to 100 nM, and the solutions without the presence of the antibody were added in duplicate, and the plates were incubated for 1 hour at room temperature. The plates were then washed to remove the unbound antibodies using an AquaMax2000 plate washer (MDS Analytical Technologies). The plate-bound antibodies were detected with a SULFO-TAG™-conjugated anti-human IgG antibody (MSD) for 1 hour at room temperature. After washes, the plates were developed with the Read Buffer (MSD) according to manufacturer's recommended procedure and the luminescent signals were recorded with a SECTOR Imager 6000 (MSD) instrument. The direct binding signals (in relative light units-RLU) were analyzed as a function of the antibody concentration and the data were fitted with a sigmoidal (four-parameter logistic) dose-response model using GraphPad Prism™ software. The potency of each antibody was determined by calculating EC₅₀. EC₅₀for binding HEK293-hLAG3 and HEK293-mfLAG3 cells was defined as the concentration of antibody at which 50% of the maximal binding signal is detected. The ratio of signal detected with 100 nM antibody binding to the HEK293-hLAG3 or HEK293-mfLAG3 cells compared to the HEK293 parental cells was recorded as an indication of specificity of LAG3 binding. For ratios lower than 2-fold at the highest antibody concentration (100 nM), the antibody was concluded as non-specific (NS).

Table 12: Antibody binding to cells overexpressing hLAG3 or mfLAG3

TABLE 12
Antibody binding to cells overexpressing hLAG3 or mfLAG3

| | Potency of Cell | Potency of Cell | Ratio of 100 nM | Ratio of 100 nM |
|---|---|---|---|---|
| | Binding to | Binding to | Antibody binding | Antibody binding |
| | HEK293-hLAG3 | HEK293-mfLAG3 | HEK293-hLAG3 to | HEK293-mfLAG3 to |
| Ab ID | Cells EC50(M) | Cells EC50(M) | HEK293 cells | HEK293 cells |
| H4sH15462P | 8.53E−10 | NS | 6.8 | NS |
| H4sH15482P | 1.13E−08 | NS | 4.5 | NS |
| H4sH15479P | 1.34E−08 | NS | 17.8 | NS |
| H4sH15498P2 | 2.87E−09 | 2.00E−08 | 99.8 | 32.7 |
| H4sH14813N | 2.03E−08 | NS | 19.8 | NS |
| H4H14813N | 1.99E−08 | NS | 8.9 | NS |
| H4H15462P | 6.85E−09 | NS | 2.3 | NS |
| H4H15482P | 2.47E−08 | NS | 7.4 | NS |
| H4H15479P | 5.20E−08 | 7.39E−08 | 20.1 | 2.1 |
| H4H15498P2 | 2.95E−09 | 9.68E−09 | 38.8 | 13.8 |
| H4H17828P2 | 1.2E−09 | 1.31E−08 | 8.7 | 6 |
| H4H17826P2 | 3.48E−09 | 7.85E−09 | 7 | 4.7 |
| H4H17823P | 1.5E−09 | 2.78E−08 | 5.3 | 2.8 |
| H4H15483P | 1.72E−08 | NS | 6.6 | NS |
| H4H17819P | 6.9E−10 | 4.02E−08 | 22.7 | 8.3 |
| H4H isotype Control | NS | NS | NS | NS |
| H4sH isotype Control | NS | NS | NS | NS |
| Comparator 1 | 4.03E−09 | NS | 7.7 | NS |
| Comparator 2 | 6.77E−09 | NS | 8.1 | NS |

NS = Non-Specific-ratio at 100 nM ab <2

As shown in Table 12, the potency of the antibodies ranged from EC₅Ovalues of 690pM to 52 nM for binding to HEK293-hLAG3 cells, with all of the antibodies having specific binding of >2-fold binding to human LAG3 expressing cells compared to parental HEK293 cells with 100 nM antibody binding. Only seven of the antibodies bound specifically to HEK293-mfLAG3 cells with EC₅Ovalues ranging from 7.9 nM to 74 nM. The comparator binding potencies on HEK293-hLag3 cells were approximately 4.0 nM and 7.0 nM. The comparators did not show specific binding to HEK293-mfLag3 cells. Irrelevant controls binding to the HEK293-hLAG3 and HEK293-mfLAG3 cells were non-specific as expected, with H4H isotype control and the H4sH isotype control binding with ratios ranging from 0.7-1.1-fold binding above the HEK293 parental cells.

### Example 7: Blocking of LAG3 binding to MHC class II in a cell adherence assay

A cell adherence assay was utilized to measure the ability of anti-LAG3 monoclonal antibodies to block either human MHCII or mouse MHCII expressing cells (RAJI and A20 cells, respectively) from adhering to hLAG3-expressing HEK293 cells attached on a microtiter plate (based on an assay described in Eur J Immunol. 1995, 25: 2718-21 by Huard, et al.).

The HEK293 (ATCC, #CRL-1573) stable cell lines were generated by lentivirus transduction to express recombinant hLAG3 (NCBI Accession No. NP_002277.4) or mouse LAG3 (mLAG3) (GenBank Accession No. NP_032505.1). The parental HEK293 cell line has no detectable expression of human LAG3 by fluorescence activated cell sorting (FACS) technology and was used to demonstrate the requirement of LAG3 for the adherence of MHCII expressing RAJI and A20 cells.

Experiments were carried out according to the following procedure: Subconfluent HEK293-hLAG3 expressing cells were washed once with 1×PBS, trypsinized, and centrifuged at 1,200 rpm for 5 minutes. Cells pellets were resuspended in 1×PBS and subsequently counted to determine cell number. The appropriate number of cells were isolated and resuspended in complete media (DME+10% FBS+ penicillin/streptomycin/glutamine), such that each well would contain 100 μL of 1.2\hat{ }4 cells. Cells were plated into sterile Nunclon™ Delta 96-Well MicroWell™ Black Optical Bottom Plates (Thermo Scientific). The wells from the perimeter of the plate were not included in the assay design to avoid edge effects. Instead of cells, 100 μL of 1×PBS was added to the perimeter wells. Plated HEK293-hLAG3 cells were incubated overnight at 37° C., at 5% CO₂. After incubation these cells were treated with anti-LAG3 antibodies and isotype controls.

On the day of treatment, all antibodies were serially (1:3) diluted in RPMI media (without FBS or supplements added). The 9-point dilution ranged between 45pM to 300 nM, with the last dilution point containing no mAb. For the testing of the H4sH antibodies in the adherence assay using RAJI cells the 9-point dilution ranged between 23pM to 150 nM, with the last dilution point containing no mAb. A final volume of 50 μL of anti-LAG3 antibodies or isotype controls were added to HEK293-hLAG3 cells and incubated for 1 hour at 37° C., at 5% CO₂. While the plated HEK293-LAG3 cells were incubating with the test or control antibodies, non-adherent B-cells (RAJI or A20) were labeled with Calcein AM using the following procedure. RAJI and A20 suspension cells were harvested and centrifuged at 1,200 rpm for 5 minutes. Cell pellets were resuspended in 1×PBS and counted. Cells were washed with RPMI, and resuspended in RPMI at a concentration of 10⁶ cells per 1 ml. Cells were labeled by adding 5 μL of Calcein AM per 1 ml of cell suspension and incubated for 30 min at 37° C., at 5% CO₂. Labeled cells were washed 1× with RPMI, centrifuged, washed with 1×PBS and resuspended in PBS at a concentration of 5×10⁶ cells per 1 ml. Subsequently, 10 μL of BD Pharmingen Fc Block (0.5 mg/mL) was added per 1 ml of the Calcein AM labeled cells, which were incubated at RT for 10 minutes. The Calcein AM-stained, Fc-blocked RAJI or A20 cells were resuspended in RPMI to a final concentration of 3×10⁶ cells per 1 ml and subsequently used in the adherence assay.

At the end of the one hour LAG3 antibody treatment of HEK293-LAG3 cells, 50 μl of labeled RAJI or A20 cells were added to each well at the density of 1.5×10⁵ cells/well. The labeled suspension cells were incubated with the HEK293 monolayer for 1 hour at 37° C., at 5% CO₂. Non-adherent cells were washed away by gently washing the wells 4× with RPMI, blotting plates with paper towels after each wash, followed by 2× wash with PBS. A final volume of 200 μl PBS was added to each well and the Calcein AM fluorescence was read at an excitation/emission wavelength of 485 nm/535 nm on the VICTOR™ X5 Multilabel Plate Reader. The IC₅Ovalues were calculated using a four-parameter logistic equation over a 9-point response curve using GraphPad Prism. IC₅₀ was defined as the concentration of antibody at which 50% blocking of adherence of HEK cells to RAJI or A20 cells was observed.

The ability of the anti-LAG3 antibodies to block binding of LAG3 to both human and mouse MHCII was assessed using a cell based adherence assay. The assay utilized a format of florescence labeled MHCII endogenously expressing cells [human MHCII (RAJI) or mouse MHCII (A20)] binding to cells engineered to express human LAG3. The ability of LAG3 antibodies to block this interaction was evaluated and results are shown in Table 13.

TABLE 13
Anti-LAG3 antibody IC₅₀ values for blocking RAJI
and A20 cell adherence to HEK293-hLAG3 cells

| | Adherence Assay | |
|---|---|---|
| | RAJI | A20 |
| Ab ID# | IC50 [M] | IC50 [M] |
| H4sH15462P | 2.50E−09 | 3.60E−09 |
| H4H15462P | 3.20E−09 | 7.10E−09 |
| H4sH15479P | 9.60E−09 | 6.30E−09 |
| H4H15479P | 3.10E−08 | 1.10E−08 |
| H4sH15482 | 3.30E−09 | 3.80E−09 |
| H4H15482P | 1.50E−08 | 1.10E−08 |
| H4sH15498P2 | 1.60E−08 | 2.10E−08 |
| H4H15498P2 | 1.50E−08 | 3.00E−08 |
| H4sH14813N | 6.00E−09 | 1.00E−08 |
| H4H14813N | 1.70E−08 | 1.00E−08 |
| H4H17828P2 | 1.10E−08 | 1.60E−08 |
| H4H17826P2 | 2.70E−08 | 1.00E−08 |
| H4H17823P | 8.60E−09 | 9.40E−09 |
| H4H15483P | 1.90E−08 | 1.60E−08 |
| H4H17819P | 1.70E−08 | 1.60E−08 |
| Isotype control | — | — |
| Comparator 1 | 3.40E−09 | 5.10E−09 |
| Comparator 2 | 7.20E−09 | 9.60E−09 |

As shown in Table 13, isotype control did not demonstrate any measurable blocking of RAJI or A20 cell binding to HEK293-hLAG3 cells. All the LAG3 antibodies of the invention blocked 92-98% of RAJI or A20 cell adherence to HEK293-hLAG3 cells at the top antibody concentration, with IC50 values ranging from 2.5 nM to 31 nM for RAJI cell adherence and 3.6 nM to 30 nM for A20 cell adherence to HEK293-hLAG3 cells. The IC50 values for the comparators were 3.4 nM and 7.2 nM for RAJI cell adherence and 5.1 nM and 9.6 nM for A20 cell adherence to HEK293-Lag3 cells.

### Example 8: Blocking of LAG3-Induced T Cell Down-Regulation in a T Cell/APC Luciferase Reporter Assay

T cell activation is achieved by stimulating T cell receptors (TCR) that recognize specific peptides presented by major histocompatibility complex class I or II (MHCI or MHCII) proteins on antigen-presenting cells (APC) (Goldrath et al. 1999, Nature 402, 255-262). Activated TCRs in turn initiate a cascade of signaling events that can be monitored by reporter genes driven by transcription factors such as activator-protein 1 (AP-1), Nuclear Factor of Activated T cells (NFAT) or Nuclear factor kappa-light-chain-enhancer of activated B cells (NFκB). T cell response is refined via engagement of co-receptors expressed either constitutively or inducible on T cells. One such receptor is LAG3, a negative regulator of T cell activity. LAG3 interacts with its ligand, MHCII, which is expressed on target cells including APCs or cancer cells, and inhibits T cell activation by shutting down positive signals initiated by the TCR signalosome (Workman et al. 2002, J Immunol 169:5392-5395).

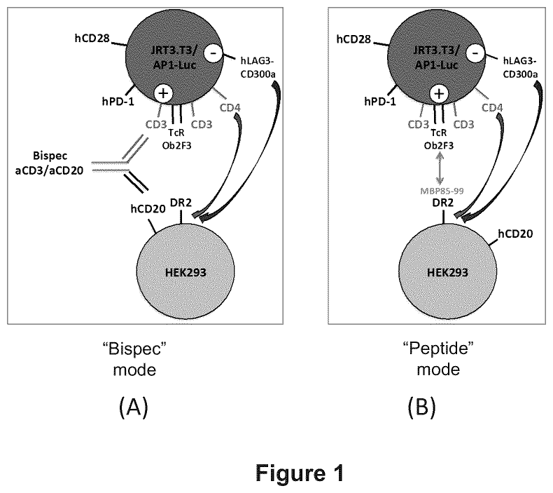

To characterize the ability of the anti-LAG3 antibodies to antagonize LAG3-mediated signaling in T cells, a LAG3 T cell/APC reporter assay was developed to measure T cell signaling induced by interaction between APC and T cells by utilizing a mixed culture derived from two mammalian cell lines: Jurkat derived T cells (clone JRT3.T3.5, as described below) and engineered APCs in HEK293 background (as described below) (FIG. 1).

For the first component, the T cell line for LAG3 T cell/APC reporter bioassay was developed as follows: A Jurkat derived T cell clone, JRT3.T3.5 (ATCC, #TIB-153) was initially transduced with the Cignal Lenti AP-1 Luc reporter (Qiagen-Sabiosciences, #CLS-011 L) as per the manufacturer's instructions. The lentivirus encodes the firefly luciferase gene under the control of a minimal CMV promoter, tandem repeats of the TPA-inducible transcriptional response element (TRE) and a puromycin resistance gene. After antibiotic selection, puromycin resistant pools of the reporter cell were further manipulated by transduction with human CD28 (NP_006130.1), Ob2F3 TCR alpha and beta subunit (Alpha AGA92550.1 Beta AAA61026.1), human LAG3 chimera (comprising the extracellular domain of human LAG3 (NP_002277.4) and trans-membrane/cytoplasmic domain of human CD300a (amino acids from 181 to 299; accession number NP 009192.2) and human PD-1 (accession number NP_005009.2). The expression of the proteins was confirmed by FACS analysis after each round of transduction. Subsequently a single clone JRT3.T3/AP1-Luc/CD28/hLAG3-CD300a chimera/hPD-1 clone 2D2 was generated and used in the cell/APC reporter bioassay. The cell line was maintained in RPMI+10% FBS+ penicillin/streptomycin/glutamine (P/S/G) supplemented with 100ug/mL hygromycin+500ug/mL G418+1 ug/mL puromycin, hereby referred to as selection medium. These cells are herein referred to as reporter T cells.

For the second component, the APC cell line for the LAG3 T cell/APC reporter bioassay was generated as follows: A stable HEK293 cell line (ATCC, #CRL-1573) expressing human CD20 (number NP_068769.2) was transduced with human with the MHCII subunits, HLA-DR2A (NP_061984.2) and HLA-DR2B (NP_002115). The HLA-DR2 positive cells were isolated by FACS and the resulting cell line, HEK293/CD20/HLA-DR2 was maintained in DME+10% +P/S/G+ non-essential amino acids supplemented with 100ug/mL hygromycin+500ug/mL G418, hereby referred to as selection medium. These cells are herein referred to as APC cells.

To engage T cell and APC in order to initiate the TCR signaling, two approaches were used: (1) Bispecific mode; and (2) Peptide pulsing mode. In this bioassay, the anti-LAG3 antibodies blocked the LAG3/MHCII interaction and rescued T cell activity by disabling the inhibitory signaling delivered by the cytosolic tail of the CD300a molecule with subsequent increase in AP1-Luc signaling. T cell activation was monitored by luciferase readout.

Bispecific mode: In this mode, a bispecific antibody composed of one Fab arm that binds to CD3 on T cells and a second Fab binding to CD20 on HEK293 cells (CD3×CD20 bispecific antibody; e.g., as disclosed in US20140088295) was utilized. The presence of the bispecific molecule results in the formation of an immunological synapse and activation of the TCR complex due to clustering of the CD3 molecules on the engineered T-cells.

A day prior to screening, the engineered reporter T cells were cultured to 5×10⁵ cells/mL in selection medium and tested in the bioassay the following day. The EC₅Ovalues of anti-LAG3 antibodies were determined in the presence of a fixed concentration of CD3×CD20 bispecific antibody (100pM). Reagents were added in the following order. The anti-Lag 3 antibodies and isotype controls were serially diluted in RPM11640 supplemented with 10% FBS and penicillin/streptomycin/glutamine (assay medium). The 10-point dilution ranged between 15 pM to 100 nM with the final dilution point containing no mAb, buffer alone control). The serially diluted antibodies were added to corresponding wells in a 96 well white flat bottom plates containing a fixed concentration (100pM) of the bispecific antibody. The overnight cultured reporter T cells were resuspended at 2×10⁶/mL in assay medium, added to anti-LAG3 antibodies plus bispecific antibody containing plates and incubated for 30 minutes at 37° C./5% CO2. The final concentration of the reporter T cells was 5×10\hat{ }4 per well.

Next, the APC cells were washed 1× with D-PBS (Irvine Scientific Cat #9240), trypsinized (Specialty Media Cat #SM-2004-C) and blocked with assay medium. After centrifugation, cells were resuspended to 4×10⁵/mL in assay medium and added to plates containing T cell reporter cells incubated with the anti-LAG3 plus bispecific antibody. The final concentration of the APC cells was ×10⁴ per well. Plates were incubated for 4-6 hours at 37° C., 5% CO2. The AP1-Luc activity was detected after the addition of ONE-Glo™ (Promega, #E6051) reagent and relative light units (RLUs) were captured on a Victor luminometer. All samples were tested in duplicates.

Peptide pulsing mode using MBP85-99: TCRs recognize specific MHC/peptide complex and lead to the activation of the T cells. The Ob2F3 TCR heterodimer used in this assay has been reported to interact with the MHCII protein, HLA-DR2AB, in complex with the MBP85-99 peptide (Myelin Basic Protein NP_001020272; SEQ ID NO: 583).

A day before the screening, the engineered reporter T cells were prepared as described above for the bispecific mode. The APC cells were pulsed overnight with 0.2 M MBP85-99 peptide (Celtek HLA-DR2 MBP85-99 in DMSO, ER-15, Lot #140411, MW 1797.1 g/mol) in corresponding antibiotic containing cell culture medium at 37° C./5% CO2. The EC₅Ovalues of anti-LAG3 antibodies were determined in the presence of 1×10⁴ pulsed APCs. Reagents were added in the following order: The anti-LAG3 antibodies and controls were serially diluted in assay medium. The 10-point serial dilution of anti-LAG3 antibodies ranged between 15 pM to 100 nM with the last dilution point containing no mAb. The antibodies were added to corresponding wells in a 96 well white flat bottom plate. The overnight cultured reporter T cells were resuspended to 2×10⁶/mL in assay medium, added to plates and incubated for 30 minutes at 37C at 5% CO₂. The final concentration of the reporter T cells was 5×10⁴ T cells per well. Next the MBP85-99 peptide pulsed APC cells were washed 1× with D-PBS (Irvine Scientific Cat #9240), trypsinized (Specialty Media Cat #SM-2004-C) and blocked with assay medium. After centrifugation, cells were resuspended to 4×10⁵/mL in assay medium and added to the anti-LAG3 plus peptide pulsed reporter T cell plates. The final concentration of the APC cells was 1×10⁴ per well. Assay plates were incubated for 4-6 hours at 37° C./5% CO2. The AP1-Luc activity was detected after the addition of ONE-Glo™ (Promega, #E6051) reagent and relative light units (RLUs) were captured on a Victor luminometer. All samples were tested in duplicates.

The EC₅Ovalues were determined from a four-parameter logistic equation over a 10-point response curve using GraphPad Prism. RLU values for each screened antibody were normalized by setting the last serial dilution containing no mAb, control to 100%, which should theoretically display the maximal AP1-Luc response elicited either with the bispecific molecule (in the bispecific mode) or by the pulsed APCs with the MBP85-99 peptide. Results are summarized in Table 14.

TABLE 14
Anti-LAG3 antibodies dependent activation of
AP1-Luc signaling in LAG3 T cell/APC bioassay

| | EC₅₀ [M] | |
|---|---|---|
| Ab ID# | Peptide mode | Bispecific mode |
| hIgG4 isotype | — | — |
| H4sH15462P | 4.86E−10 | 1.48E−10 |
| H4H15462P | 3.44E−10 | 2.24E−10 |
| H4sH15479P | 1.35E−10 | 8.72E−11 |
| H4H15479P | 2.84E−10 | 1.45E−10 |
| H4sH15482P | 8.06E−10 | 5.18E−10 |
| H4H15482P | 2.48E−09 | >100 nM |
| H4sH15498P2 | 4.61E−09 | >100 nM |
| H4H15498P2 | 5.89E−09 | 6.62E−09 |
| H4sH14813N | 8.16E−10 | 6.88E−10 |
| H4H14813N | 1.01E−09 | 1.07E−09 |
| H4H15483P | 2.54E−09 | 3.69E−09 |
| H4H17819P | 2.12E−09 | >100 nM |
| H4H17823P | 6.88E−10 | >100 nM |
| H4H17826P2 | 8.83E−09 | 2.66E−07 |
| H4H17828P2 | 4.91E−09 | >100 nM |
| Comparator 1 | 2.79E−10 | 1.57E−10 |
| Comparator 2 | 3.49E−10 | 2.38E−10 |

As shown in Table 14, all the anti-Lag3 mAbs of the invention that were tested in the T cell/APC bioassay, rescued LAG3/HLA-DR2 inhibition with EC₅Ovalues ranging from 135pM to 8.83 nM in the peptide mode of the assay and with EC₅Ovalues ranging from 87pM to more than 100 nM in the bispecific mode of the assay, while the comparators exhibited EC₅Ovalues of 280 pM and 350 pM. Tested isotype controls did not show significant rescue activity in the bioassay. In a further experiment, the addition of an anti-PD-1 antibody (REGN 2810; described in Example 12 herein) did not enhance rescue of T-cell activation.

In a second experiment, the HEK293/CD20/HLA-DR2 cell line was transduced with human PD-L1 gene (amino acids 1-290 of accession number NP_054862.1) that had been cloned into a lentiviral (pLVX) vector system. As described above, Jurkat derived T cells expressing hLAG-3 and an AP-1-driven luciferase reporter were incubated with PD-L1 positive antigen-presenting cells expressing human MHC II/peptide complex. In this assay, anti-LAG-3 antibodies rescued T cell activity only in the presence of an anti-PD-1 antibody (REGN2810; described in Example 12 herein), not in the absence of the anti-PD-1 antibody. In summary, anti-LAG-3 antibodies rescued T cell activation by blocking LAG-3/MHC II inhibitory signaling and synergized with an anti-PD-1 antibody in this T cell assay.

### Example 9: Anti-LAG3 Antibodies Display Binding to CD4+ and CD8+ Stimulated Cynomolgus T-cells

In this example, the ability of selected anti-LAG3 antibodies to bind to LAG3-expressing CD4+ and/or CD8+ cynomolgus T cells via FACS was assessed.

First, T-cells were isolated from cynomolgus whole blood (Bioreclamation, Westbury, NY). Briefly, whole blood from two cynomolgus donors was diluted 1:1 in PBS and layered over 85% Ficoll-Paque solution. Following standard procedures, red blood cells were separated from mononuclear cells and T-cells were isolated using panT cells isolation kit (Miltenyi Biotech).

Isolated T-cells were activated using a T-cell Activation/Expansion kit for non-human primate (Miltenyi Biotech). The cell-bead mixture was resuspended at a concentration of 1×10⁶ cells/mL in complete media. After 72 h of incubation, beads were removed and activated T-cells were expanded in complete media for 96 h.

Next, stimulated cynomolgus T-cells from each donor, as described above, were stained with LIVE/DEAD dye (Molecular Probes) to distinguish between live and dead cells via FACS. Subsequently, cells were co-incubated with PE-conjugated anti-CD8 and FITC-conjugated anti-CD4 for 15 min on ice to enable the detection of CD4 and CD8 positive and negative cell populations.

To assess the binding of anti-LAG3 antibodies to previously isolated CD4+ and CD8+ cynomolgus T-cells, anti-LAG3 antibodies and isotype controls were serially diluted in blocking buffer and plated in a 96 well V-bottom plate with final doses ranging from 100 nM to 50 pM. Next, the serially diluted antibodies were directly labeled using Zenon Alexa-Fluor 647 (Molecular Probes) and incubated with the cynomolgus CD4+/CD8+ T-cell suspension for 15-30 min on ice. Cells were centrifuged and pellets washed with staining buffer to remove unbound antibodies and fixed for 12 h with a 1:1 dilution of Cytofix (BD Biosciences) and staining buffer. Following the incubation period, fixation buffer was removed and pellets were resuspended in staining buffer, filtered, and used for fluorescence measurements.

Fluorescence measurements were acquired on the Fortessa (Becton Dickinson) and data analyzed in FlowJo to determine the mean fluorescence intensities (MFI). EC₅Ovalues were calculated from a four-parameter logistic equation over an 11-point response curve using GraphPad Prism. The MFI ratio for each antibody was calculated by dividing the MFI at 11.1 nM by MFI at 0 nM of antibody concentration (negative control).

TABLE 15
EC₅₀ and MFI Ratios of Selected Anti-LAG3 Antibodies Binding to CD4+
and CD8+ Cynomolgus T-cells from Multiple Donors

| | Cynomolgus Donor 1 | | | | Cynomolgus Donor 2 | | | |
|---|---|---|---|---|---|---|---|---|
| FACS | CD4+ | | CD8+ | | CD4+ | | CD8+ | |
| binding on | | Ratio of | | Ratio of | | Ratio of | | Ratio of |
| Stimulated T- | | MFI | | MFI | | MFI | | MFI |
| cell | EC₅₀ | (11 nM/ | EC₅₀ | (11 nM/ | EC₅₀ | (11 nM/ | EC₅₀ | (11 nM/ |
| population | [M] | 0 nM) | [M] | 0 nM) | [M] | 0 nM) | [M] | 0 nM) |
| H4sH15482P | 1.20E−09 | 1.3 | 4.60E−10 | 3 | 3.80E−10 | 1.3 | 4.10E−10 | 4.7 |
| H4sH14813N | 4.20E−10 | 1.3 | 8.50E−11 | 3.5 | 2.20E−10 | 1.5 | 1.10E−10 | 6.3 |
| Comparator 1 | NB | 1.1 | 9.20E−10 | 2.4 | NB | 1.2 | 7.80E−10 | 4.2 |
| hIgG4 Isotype | NB | 1 | NB | 1 | NB | 1 | NB | 1 |
| Control | | | | | | | | |

NB: No binding

As previously shown, anti-LAG3 antibodies H4sH15482P and H4sH14813N displayed binding to engineered cynomolgus LAG3-HEK293 cell lines. In this example, H4sH15482P and H4sH14813N demonstrated binding specifically to CD4+ and CD8+ LAG3-expressing cynomolgus T-cells with EC₅₀'s ranging from 1.1 nM to 85 pM (Table 15). In summary, the selected anti-LAG3 antibodies described in this example binds show cross-reactivity to LAG3 expressed on cynomolgus activated CD4+ and CD8+ T-cells. In contrast, comparator 1 demonstrated binding to CD8+ cynomolgus T-cells only.

### Example 10: In vivo efficacy of anti-LAG3 antibodies against Colon26 tumors

The in vivo efficacy of anti-mouse LAG3 antibodies alone and in combination with anti-mouse PD-1 antibodies was studied in a Colon 26 preclinical syngeneic tumor model.

BALB/c mice were purchased from Taconic Biosciences. Colon 26, a mouse adenocarcinoma cell line originated from BALB/c mouse strain, was purchased from American Type Culture Collection (ATCC). Fifty BALB/c mice were subcutaneously implanted with 1×10⁶ Colon 26 (mouse adenocarcinoma line originated from BALB/c strain) cells on Day 0. On Day 8, forty mice with an average tumor volume of 50 mm³ were selected and randomized into 4 treatment groups (N=10/group). On days 14, 17, 21, 24 and 28, mice were dosed with antibodies as follows: Group 1, rat IgG2a isotype control (clone 2A3, BioXCell, Catalog #BE0089) and mlgG1 isotype control (clone MOPC-21, BioXCell, Catalog #BE0083) antibodies; Group 2, anti-mouse PD-1 antibodies (rat IgG2a anti-mouse PD-1 antibody, clone RPMI-14, BioXCell, catalog #BE0089) and mlgG1 isotype control antibodies; Group 3, anti-mouse LAG3 antibodies (rat IgG1 anti-mouse LAG3 antibody, clone C9B7W, BioXCell, Catalog #BE0174) and rat IgG2a control antibodies; Group 4, anti-mouse PD-1 and anti-mouse LAG3 antibodies. All antibodies were administered by intraperitoneal injection at 10 mg/kg. Tumor volumes were monitored by caliper measurement twice per week for the duration of the experiment (42 days).

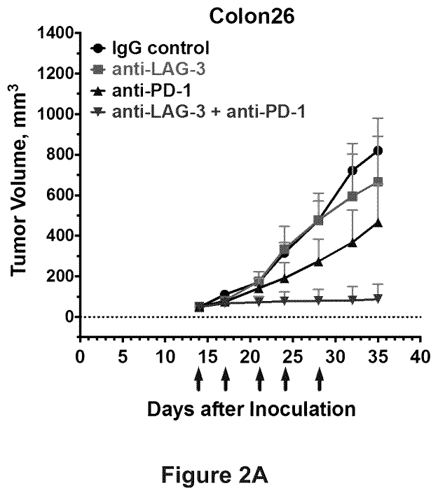

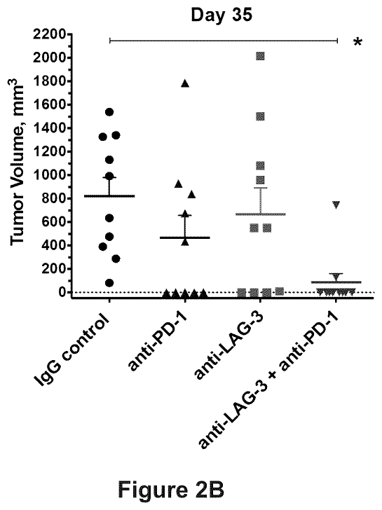

A significant reduction of tumor growth was observed in mice treated with the anti-mouse PD-1 antibody (FIGS. 2A-B), with 5 out of 10 mice becoming tumor-free by day 45 at the end of experiment. Tumor growth reduction was also observed in a subset of mice treated with the anti-mouse LAG3 antibody, with 3 out of 10 mice becoming tumor-free by day 45. In striking contrast, combination treatment with anti-LAG3 and anti-PD-1 antibodies was superior to either monotherapy, with 8 out of 10 mice becoming tumor-free at the end of experiment, suggesting a potent additive effect of anti-LAG3 and anti-PD-1 treatment. There were no tumor-free mice at the end of the experiment in the control group. By day 35, there was a statistically significant reduction of tumor volumes in mice treated with combination therapy (p<0.05, FIG. 2B) but not with either monotherapy. Despite efficient tumor clearance, no evidence of body weight loss was observed (not shown). Thus, in an established Colon 26 tumor model, a combination regimen of anti-PD-1 and anti-LAG3 antibodies was significantly more efficacious than the corresponding monotherapies.

The in vivo activity of anti-mouse PD-1 and anti-mouse LAG3 antibodies was also studied in 4T1, RENCA and A20 preclinical syngeneic tumor models. Combination treatment with both antibodies resulted in at least an additive anti-tumor effect compared to either single antibody treatment (data not shown).

### Example 11: In Vivo Efficacy of Anti-Mouse LAG3 Antibodies Against MC38 Tumors

The effect of PD-1 and LAG3 dual blockade was examined against established MC38 tumors in C57BL/6 mice using anti-mouse PD-1 and anti-mouse LAG3 blocking antibodies.

C57BL/6 mice were purchased from Taconic Biosciences. MC38, a mouse adenocarcinoma cell line originated from C57BL/6 mouse strain, was obtained from the National Health Institutes depository. The anti-LAG3 and anti-PD-1 antibodies as well as controls were obtained as described in Example 10.

Fifty C57BL/6 mice were subcutaneously implanted with 3×105 MC38 cells in the flank on Day 0. On Day 8, forty mice with an average tumor volume of 45 mm³ were selected and randomized into 4 treatment groups (N=10/group). On days 8, 12, 15, 19 and 23, mice were dosed with antibodies as follows: Group1, rat IgG2a and mIgG1 isotype control antibodies; Group 2, anti-mouse PD-1 and mlgG1 antibodies; Group 3, anti-mouse LAG3 and rat IgG2a antibodies; Group 4, anti-mouse PD-1 and anti-mouse LAG3 antibodies. All antibodies were administered at 10 mg/kg by intraperitoneal injection. Tumor volumes were monitored by caliper measurement twice per week for the duration of the experiment (28 days).

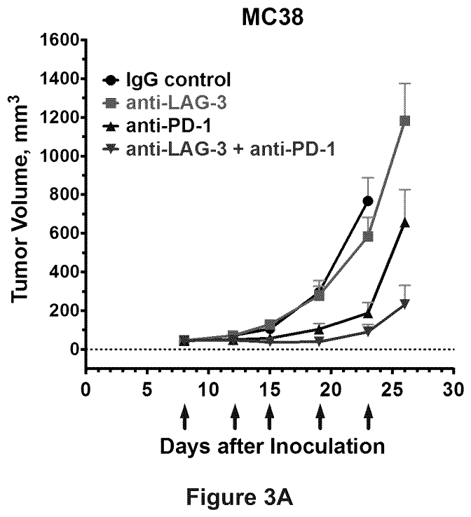

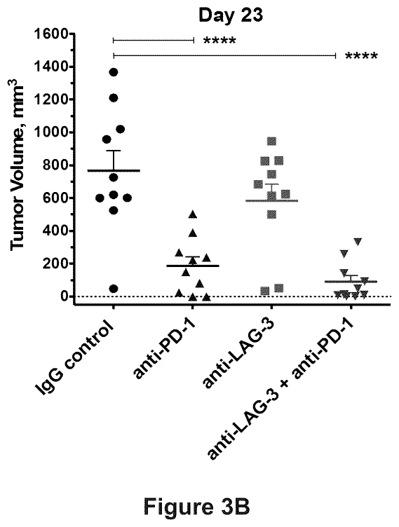

Anti-PD-1 antibody therapy significantly reduced tumor growth in a subset of mice (p<0.0001; day 23) (FIGS. 3A-B), with 2 out of 10 mice becoming tumor-free at the end of experiment. Anti-LAG3 antibody monotherapy did not show any significant anti-tumor effect (0/10 mice tumor-free at the end of the experiment), whereas the combination of anti-LAG3 and anti-PD-1 antibodies was superior to anti-PD-1 therapy alone indicating a synergistic effect.

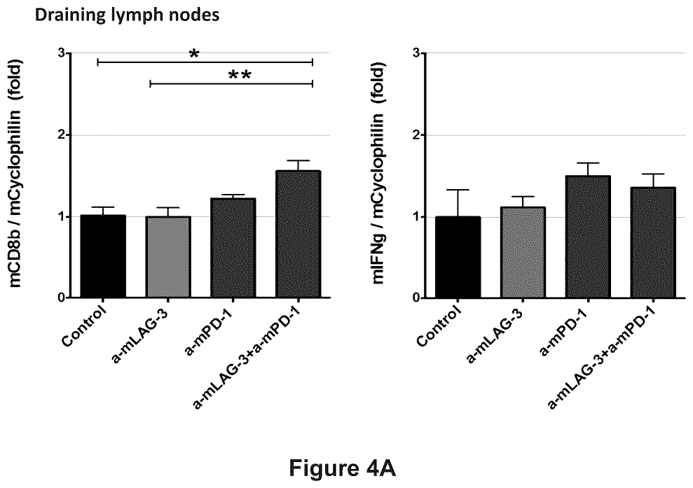

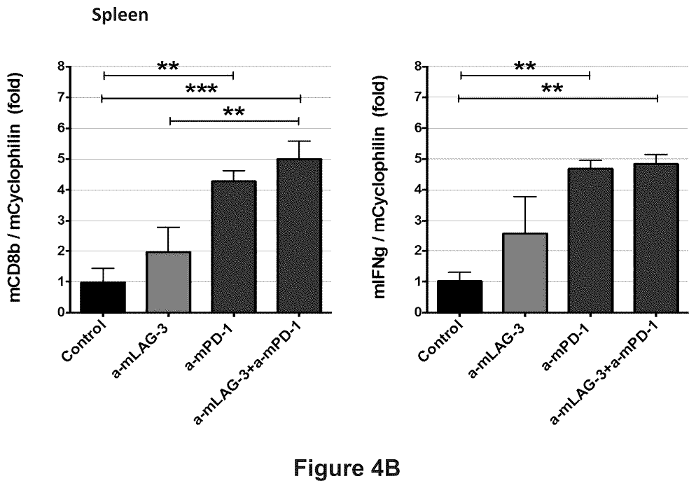

To test the immunomodulatory properties of anti-PD-1 and anti-LAG3 antibodies in combination, T-cell markers in spleens and draining lymph nodes (DLN) of the treated mice were examined (FIG. 4). Spleens and draining lymph nodes of tumor bearing mice were removed by dissection and placed into 15 mL RPMI1640 Glutamax I medium (Invitrogen). Total RNA was isolated and RT-PCR Taqman analysis was performed using a primer/probe mix specific for mouse CD83 [probe: AGCAGCTCTGCCCTCAT (SEQ ID NO: 584), forward primer: GCTCTGGCTGGTCTTCAGTATG (SEQ ID NO: 585), reverse primer: TTGCCGTATGGTTGGTTTGAAC (SEQ ID NO: 586)] and mouse IFNγ (Mm01168134_ml, Applied Biosystems). Samples were normalized to mouse cyclophilin B transcript expression levels.

TaqMan analysis demonstrated CD8+ T cell expansion in both the DLN and spleens of mice in the combination treatment group, as well as increased production of T cell effector molecule IFNγ in both combination and single anti-PD-1 treated animals, indicating that PD-1 and LAG3 blockade increases proliferation and effector function of T cells.

### Example 12: In vivo efficacy of anti-human LAG3 antibodies against MC38 tumors

In this experiment, the efficacy of an exemplary anti-human LAG3 antibody of the invention against MC38 tumors was studied. Anti-human LAG3 mAbs do not bind to mouse LAG3. Therefore, to study the anti-tumor properties of these antibodies in vivo in mice, mice humanized to express human LAG3 protein were used. For the experiments herein, double-humanized mice that express the extracellular portion of human PD-1 and of human LAG3 and the transmembrane and intracellular portions of the mouse versions of these proteins were generated using VelociGene® technology (Valenzuela et al 2003; Nat. Biotechnol. 21: 652-659).

Double humanized LAG3/PD-1 mice were engineered using VelociGene® technology to replace the extracellular domains of mouse Pdcd1 and Lag3 genes with the corresponding regions of human PD-1 and human LAG3 genes (U.S. Patent Application Ser. No. 62/258,181, filed on Nov. 20, 2015). Humanized PD-1 and LAG3 expression was validated by examining PD-1 and LAG3 protein expression on humanized mouse T cells after anti-CD3/anti-CD28 antibody stimulation. Human PD-1 and LAG3 protein interaction with the corresponding mouse ligands was verified by testing binding of human LAG3 to mouse MHC II in a cell adhesion assay, and binding of human PD-1 to mouse PD-L1 in SPR-Biacore binding study.

The exemplary anti-LAG3 antibody used for this study is a fully human antibody that binds specifically to human LAG3 and comprises HCDR1-HCDR2-HCDR3-LCDR1-LCDR2-LCDR3 of SEQ ID NOs: 420-422-424-428-430-432 and HCVR/LCVR of SEQ ID Nos: 418/426 (also known as H4sH15482P and hereinafter “mAb1”).

Anti-human PD-1 antibodies are disclosed in US Patent Application publication US20150203579, hereby incorporated by reference in its entirety. The anti-PD-1 antibody used in this Example is H4H7798N (as disclosed in US20150203579; also known as REGN2810. More specifically, H4H7798N comprises a heavy chain of SEQ ID NO: 330 and a light chain of SEQ ID NO: 331 as disclosed in US20150203579). REGN2810 (anti-hPD-1) binds with high affinity to human PD-1 and blocks PD-1 interaction with PD-L1 and PD-L2.

Mouse colon adenocarcinoma cells (MC38.Ova) were engineered to express chicken ovalbumin in order to increase tumor immunogenicity.

LAG3^{hum/hum} PD_1^{hum/hum} mice were subcutaneously implanted with 1.5×10⁶ MC38.Ova cells on day 0 and randomized into three treatment groups (N=7/group). On days 3, 6, 10, 14 and 17 mice were administered mAb1 (anti-hLAG3) (25 mg/kg), REGN2810 (anti-hPD-1) (10 mg/kg) or isotype control antibody (25 mg/kg) by intraperitoneal injection. Tumor volumes were monitored by caliper measurement twice per week for the duration of the experiment (24 days).

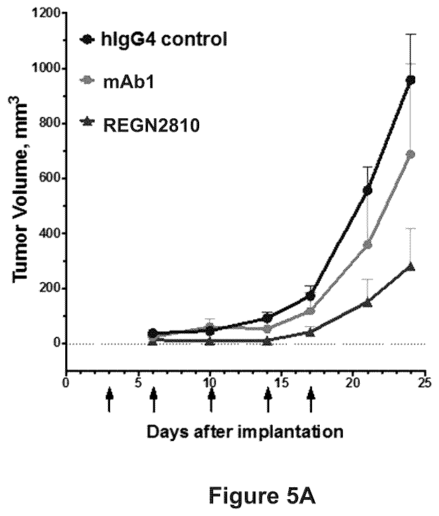

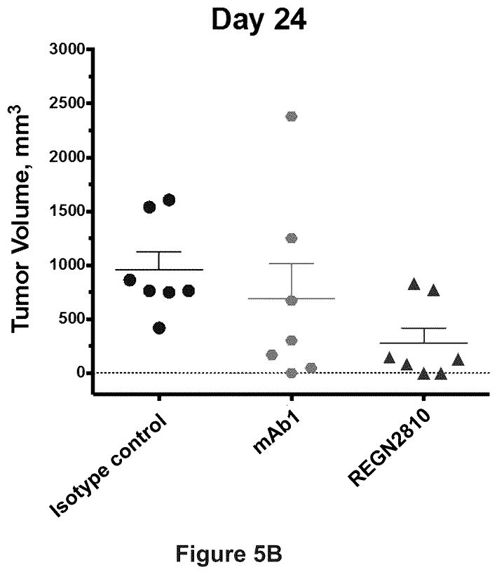

LAG3^{hum/hum} PD-1^{hum/hum} double-humanized mice were inoculated with MC38.Ova cells on day 0 and dosed with mAb1 (anti-hLAG3) (25 mg/kg), REGN2810 (anti-hPD-1) (10 mg/kg) or isotype control (25 mg/kg) via intraperitoneal route on days 3, 6, 10, 14 and 17. REGN2810 (anti-hPD-1) showed partial tumor growth inhibition (FIG. 5), with tumor regression observed in 2 out of 7 mice, whereas none of the mice were tumor-free in isotype control group. mAb1 (anti-hLAG3) monotherapy at 25 mg/kg did not show any significant effect on tumor growth, with only 1 out of 7 mice becoming tumor-free by the end of the experiment. Mouse body weights were not affected by mAb1 (anti-hLAG3), REGN2810 (anti-hPD-1) or isotype control treatments.

### Example 13: In Vivo Efficacy of a Combination of Anti-Human LAG3 and Anti-Human PD-1 Antibodies Against MC38 Tumors

In this experiment, the efficacy of mAb1 (anti-hLAG3) in combination with REGN2810 (anti-hPD-1) was examined against MC38.Ova tumors in double-humanized LAG3^{hum/hum} PD-1^{hum/hum}mice. Double-humanized mice are described in Example 12 herein.

The exemplary anti-LAG3 antibody used for this study is a fully human antibody that binds specifically to human LAG3 and comprises HCDR1-HCDR2-HCDR3-LCDR1-LCDR2-LCDR3 of SEQ ID NOs: 420-422-424-428-430-432 and HCVR/LCVR of SEQ ID Nos: 418/426 (also known as H4sH15482P and hereinafter “mAb1”).

The anti-PD-1 antibody used in this Example is H4H7798N (as disclosed in US20150203579; also known as REGN2810). REGN2810 (Anti-hPD-1) binds with high affinity to human PD-1 and blocks PD-1 interaction with PD-L1 and PD-L2.

Mice were subcutaneously implanted with 1×10⁶ MC38.Ova cells on day 0 and randomized into four treatment groups (N=7 in the control group, and N=12 each in mAb1 (anti-hLAG3), REGN2810 (anti-hPD-1), and mAb1 (anti-hLAG3)+ REGN2810 (anti-hPD-1) combination treatment groups). On days 3, 7, 10, 14 and 17, mice were administered mAb1 (anti-hLAG3) (25 mg/kg), REGN2810 (anti-hPD-1) (10 mg/kg), combination of mAb1 (anti-hLAG3) (25 mg/kg) and REGN2810 (anti-hPD-1) (10 mg/kg), or isotype control (25 mg/kg) by intraperitoneal injection. Tumor volumes were monitored by caliper measurement twice per week for the duration of the experiment (32 days) and tumor free animals were monitored for the absence of tumor recurrence for up to 80 days.

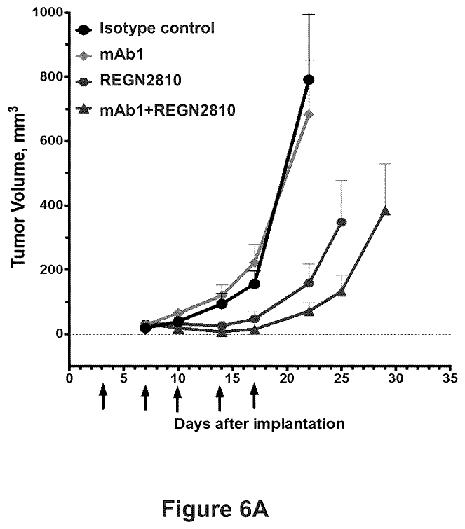

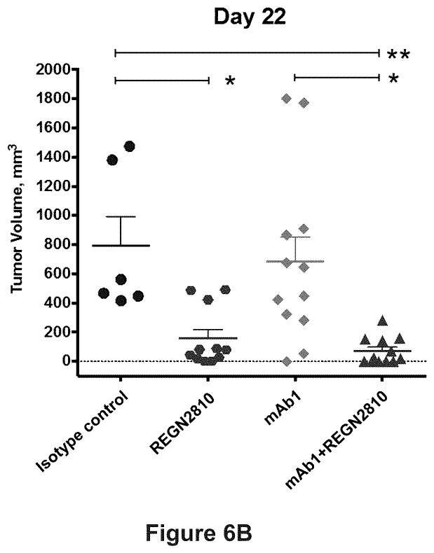

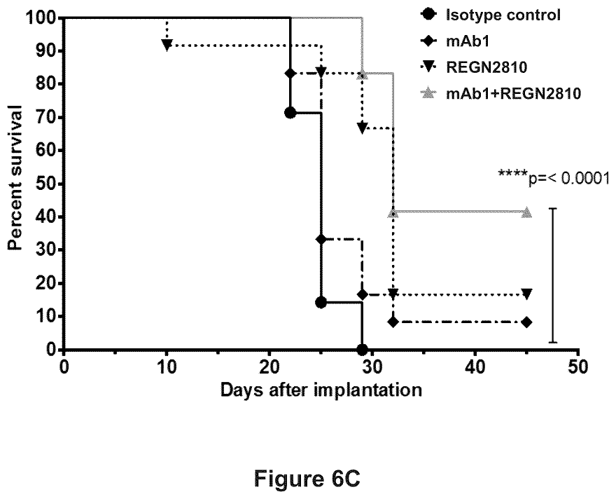

REGN2810 (anti-hPD-1) monotherapy resulted in tumor growth inhibition (FIGS. 6A-B), with tumor regression in 2 out of 12 (17%) animals, whereas mAb1 (anti-hLAG3) was not significantly efficacious, confirming the finding of the previous experiment. Tumor regression was only observed in 1 out of 12 mice treated with mAb1 (anti-hLAG3). In the isotype control group, there were no tumor-free mice on day 32. By contrast, combination of mAb1 (anti-hLAG3) and REGN2810 (anti-hPD-1) demonstrated robust inhibition of MC38.Ova tumor growth, resulting in 5 out of 12 (42%) tumor-free mice by the end of experiment. None of the tumor-free mice showed tumor recurrence for 80 days post-implantation, indicating long-lasting effects of combination immunotherapy. One-way ANOVA with Tukey's multiple comparison post-test revealed a significant difference in tumor volumes between the mAb1 (anti-hLAG3) and REGN2810 (anti-hPD-1) combination treatment groups compared to mAb1 (anti-hLAG3) monotherapy (p<0.05) or isotype control (p<0.01) on day 22 (FIG. 6B). REGN2810 (anti-hPD-1) monotherapy also showed a significant reduction of tumor growth relative to (p<0.05). mAb1 (anti-hLAG3) and REGN2810 (anti-hPD-1) combination treatment also resulted in a significant improvement in animal survival rate (p<0.0001), as analyzed by log-rank (Mantel-Cox) test (FIG. 6C). Despite improved efficacy of the combination therapy, no evidence of body weight loss was observed (data not shown). In summary, combination of mAb1 (anti-hLAG3) and REGN2810 (anti-hPD-1) resulted in improved efficacy including reduced tumor growth, improved tumor clearance and improved survival compared to REGN2810 (anti-hPD-1) or mAb1 (anti-hLAG3) monotherapies.

### Example 14: Dose-ranging studies of a combination of anti-LAG3 and anti-PD-1 antibodies against MC38 tumors

This Example describes a set of dose-ranging experiments measuring in vivo efficacy of a combination of anti-human PD-1 and anti-human LAG3 antibodies against MC38.Ova tumors in double-humanized LAG3^{hum/hum} PD1^{hum/hum} mice. Double-humanized mice are described in Example 12 herein.

The exemplary anti-LAG3 antibody used for this study is a fully human antibody that binds specifically to human LAG3 and comprises HCDR1-HCDR2-HCDR3-LCDR1-LCDR2-LCDR3 of SEQ ID NOs: 420-422-424-428-430-432 and HCVR/LCVR of SEQ ID Nos: 418/426 (also known as H4sH15482P and hereinafter “mAb1”).

The anti-PD-1 antibody used in this Example is H4H7798N (as disclosed in US20150203579; also known as REGN2810). REGN2810 (Anti-hPD-1) binds with high affinity to human PD-1 and blocks PD-1 interaction with PD-L1 and PD-L2.

In a first experiment, REGN2810 (anti-hPD-1) was titrated and tested at 10 mg/kg and 1 mg/kg dose levels, both dose groups as a single agent and in combination with mAb1 (anti-hLAG3) (25 mg/kg). The results of both combination treatment groups were compared to REGN2810 (1 mg/kg or 10 mg/kg) or mAb1 (25 mg/kg) monotherapy to determine if an additive/synergistic anti-tumor effect is observed. Treatment with isotype control antibody (25 mg/kg) was also tested. LAG-3hum/humPD-1 hum/hum mice were inoculated with MC38.Ova cells on day 0 and randomized into six treatment groups (N=6 in the isotype control group and N=12 in mAb1, REGN2810, or REGN2810+ mAb1 treatment groups). Mice were administered: mAb1 (25 mg/kg); REGN2810 (10 mg/kg); REGN2810 (1 mg/kg); mAb1 (25 mg/kg)+ REGN2810 (10 mg/kg); mAb1 (25 mg/kg)+ REGN2810 (1 mg/kg); or isotype control (25 mg/kg), via intraperitoneal injection on days 3, 7, 10, 14 and 17. Tumor volumes were monitored for 36 days, and tumor-free animals were monitored for up to 80 days.

Table 16 shows the mean tumor volumes at various time-points in the study and the number of tumor-free mice at Day 36 is shown in Table 17.

TABLE 16
Mean tumor volumes at various time-points

| | Tumor Volume, mm³ Mean (±SEM) | | | | | | | | |
|---|---|---|---|---|---|---|---|---|---|
| Treatment Group | Day 7 | Day 10 | | Day 14 | | Day 17 | | Day 22 | |
| Isotype Control | 24 (±6) | 54 | (±11) | 104 | (±25) | 269 | (±70) | 749 | (±180) |
| mAb1 (anti-LAG3) | 27 (±4) | 53 | (±20) | 83 | (±38) | 155 | (±73) | 451 | (±211) |
| (25 mg/kg) | | | | | | | | | |
| R2810 (10 mg/kg) | 27 (±4) | 29 | (±8) | 34 | (±11) | 86 | (±26) | 313 | (±98) |
| R2810 (1 mg/kg) | 36 (±8) | 92 | (±23) | 150 | (±47) | 264 | (±73) | 669 | (±183) |
| R2810 (10 mg/kg) + mAb1 | 25 (±3) | 10 | (±3) | 4 | (±4) | 8 | (±5) | 38 | (±19) |
| R2810 (1 mg/kg) + mAb1 | 31 (±7) | 38 | (±13) | 55 | (±35) | 106 | (±58) | 378 | (±137) |

TABLE 17
Number of Tumor-free Mice on Day 36

| | Tumor-free Mice (Day 36) | |
|---|---|---|
| Group | n/N^{a} (%) | |
| Isotype Control | 0/6 | (0%) |
| REGN2810 (1 mg/kg) | 2/12 | (16%) |
| REGN2810 (10 mg/kg) | 4/12 | (33%) |
| mAb1 (25 mg/kg) | 1/12 | (8%) |
| REGN2810 (1 mg/kg) + mAb1 (25 mg/kg) | 3/12 | (25%) |
| REGN2810 (10 mg/kg) + mAb1 (25 mg/kg) | 6/12 | (50%) |

^{a}n/N: # tumor-free mice/total mice per group

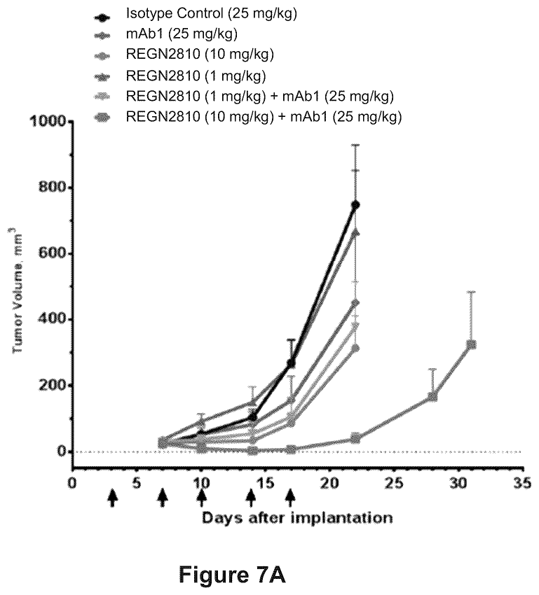

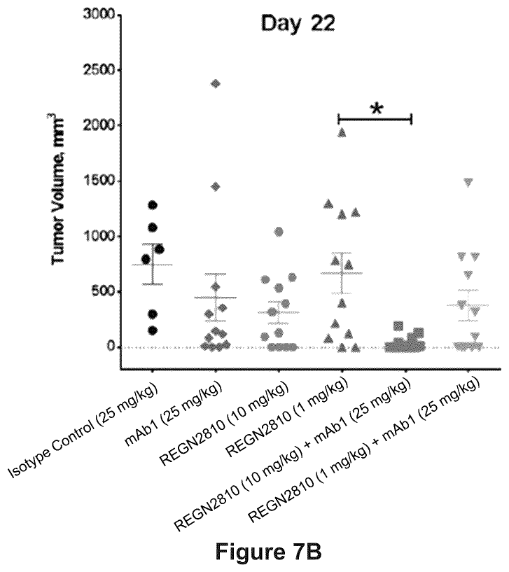

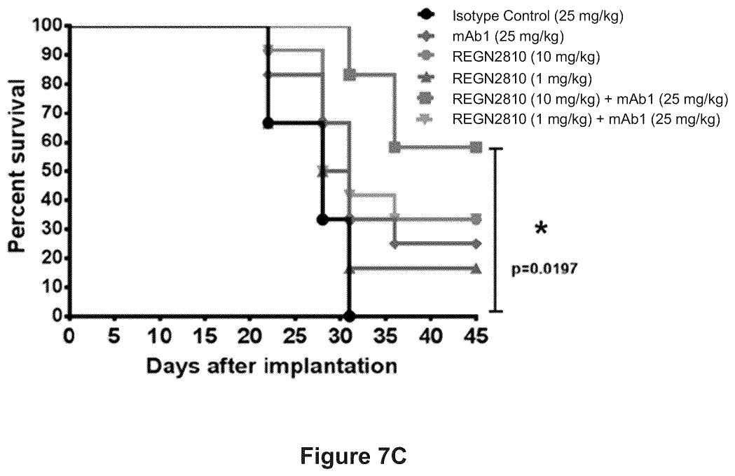

REGN2810 monotherapy at 1 mg/kg only showed minimal tumor regression over 36 days, similar to results for the isotype control treatment group (FIG. 7A-B). 2/12 (˜16%) and 0/6 (0%) mice were tumor-free at Day 36 in the REGN2810 (1 mg/kg) and the isotype control groups, respectively. In contrast, mAb1 (25 mg/kg), REGN2810 (10 mg/kg) and REGN2810 (1 mg/kg)+ mAb1 (25 mg/kg) treatment groups demonstrated a similar reduction in tumor growth over 22 days (FIG. 7A). On Day 36, mAb1 (25 mg/kg) treatment resulted in 1/12 (˜8%) mice tumor-free, whereas 4/12 (˜33%) and 3/12 (25%) mice were tumor-free in the REGN281 0 (10 mg/kg) group and REGN2810 (1 mg/kg)+ mAb1 (25 mg/kg) group, respectively. Overall, mAb1 (25 mg/kg)+ REGN281 0 (10 mg/kg) demonstrated the most robust reduction in MC38.Ova tumor growth, with 6 out of 12 (50%) mice becoming tumor-free by Day 36. None of the tumor-free mice showed tumor recurrence for 80 days post-implantation, despite the cessation of treatment, indicating long-lasting effects of combination immunotherapy. mAb1 (25 mg/kg)+ REGN281 0 (10 mg/kg) treatment also resulted in a significant improvement in animal survival rate (p=0.0197), as analyzed by log-rank (Mantel-Cox) test (FIG. 7C). Also, no evidence of body weight loss was observed.

In a second experiment, mAb1 (anti-hLAG3) was titrated and tested at 25 mg/kg and 5 mg/kg dose levels, both dose groups as a single agent and in combination with REGN2810 (anti-hPD-1) (10 mg/kg). The results of both combination treatment groups were compared to mAb1 (5 mg/kg or 25 mg/kg) or REGN2810 (10 mg/kg) monotherapy to determine if an additive/synergistic anti-tumor effect is observed. Treatment with isotype control antibody (25 mg/kg) was also tested. LAG-3hum/humPD-1 hum/hum mice were inoculated with MC38.Ova cells on day 0 and randomized into six treatment groups (N=6 in the isotype control group and N=12 in mAb1, REGN2810, or REGN2810+ mAb1 treatment groups). Mice were administered: mAb1 (5 mg/kg); mAb1 (25 mg/kg); REGN2810 (10 mg/kg); mAb1 (5 mg/kg)+ REGN2810 (10 mg/kg); mAb1 (25 mg/kg)+ REGN2810 (10 mg/kg); or isotype control (25 mg/kg), via intraperitoneal injection on days 3, 7, 10, 14 and 17. Tumor volumes were monitored for 31 days.

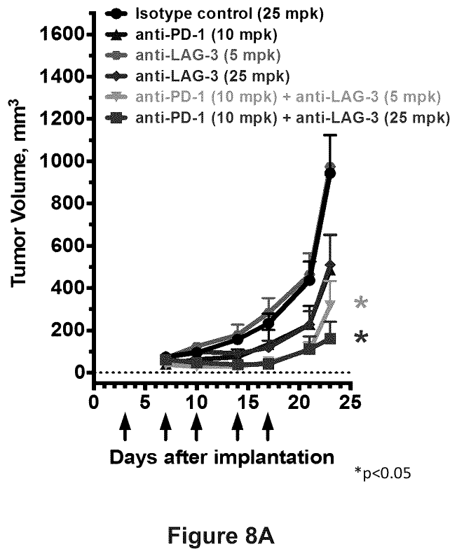

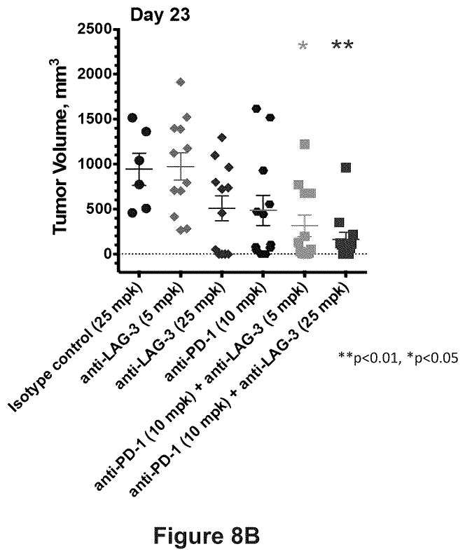

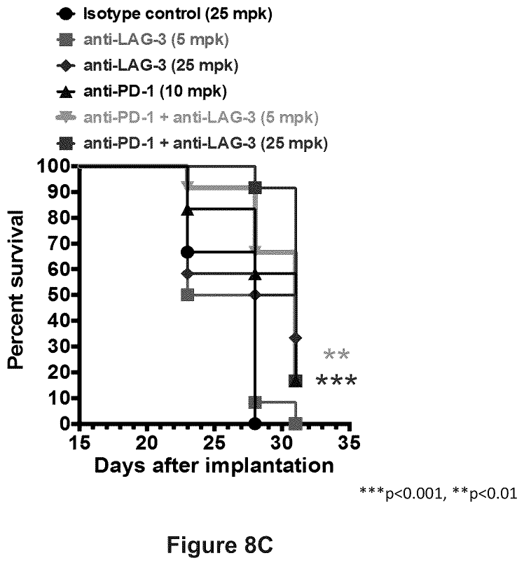

mAb1 monotherapy at 5 mg/kg showed minimal tumor regression similar to isotype control treatment group. The combination of mAb1 (25 mg/kg)+ REGN2810 (10 mg/kg) showed the most robust tumor reduction (FIG. 8A). A potent combination effect was also demonstrated by the mAb1 (5 mg/kg)+ REGN2810 (10 mg/kg) treatment group. Tumor volumes as measured on Day 23 are shown in FIG. 8B. The combination of anti-LAG-3 antibody and REGN2810 also showed a significant improvement in animal survival (p<0.001, as analyzed by log rank Mantel-Cox test) (FIG. 8C). The results show that a potent combination effect is seen even at low anti-LAG3 antibody doses. Based on the results shown herein, it may not be necessary to use high doses of anti-LAG3 antibodies in a combination with anti-PD-1 antibodies in treatment regimens against tumors.

In summary, combination of REGN2810 (anti-hPD-1) and mAb1 (anti-hLAG3) in MC38.ova tumor model in double humanized LAG3/PD-1 mice, which allows testing of clinical antibodies that do not cross to mouse receptors, demonstrated improved dose-dependent efficacy, including reduced tumor growth and improved survival, compared to REGN2810 (anti-hPD-1) and mAb1 (anti-hLAG3) monotherapies. Robust anti-tumor efficacy of REGN2810 (anti-hPD-1) and mAb1 (anti-hLAG3) combination in preclinical setting supports their clinical development as a combination cancer immunotherapy.

### Example 15: In vivo efficacy of a combination of anti-human LAG3 and anti-human PD-1 antibodies against established MC38 tumors

In this experiment, the efficacy of mAb1 (anti-hLAG3) in combination with REGN2810 (anti-hPD-1) was examined against established MC38.Ova tumors in double-humanized LAG3^{hum/hum} PD-1^{hum/hum} mice. Double-humanized mice are described in Example 12 herein.

The exemplary anti-LAG3 antibody used for this study is a fully human antibody that binds specifically to human LAG3 and comprises HCDR1-HCDR2-HCDR3-LCDR1-LCDR2-LCDR3 of SEQ ID NOs: 420-422-424-428-430-432 and HCVR/LCVR of SEQ ID Nos: 418/426 (also known as H4sH15482P and hereinafter “mAb1”).

The anti-PD-1 antibody used in this Example is H4H7798N (as disclosed in US20150203579; also known as REGN2810). REGN2810 (Anti-hPD-1) binds with high affinity to human PD-1 and blocks PD-1 interaction with PD-L1 and PD-L2.

LAG-3^{hum/hum} PD-1^{hum/hum} mice were inoculated with MC38.Ova cells subcutaneously on day 0. On day 10 mice with an average tumor volume of 100 mm³ were selected and randomized into four treatment groups. Mice were administered mAb1 (25 mg/kg; N=9), REGN2810 (10 mg/kg; N=10), mAb1 (25 mg/kg)+ REGN2810 (10 mg/kg) combination (N=11) or isotype control antibody (25 mg/kg; N=7) by IP injection on days 10, 14, 17, 22. Tumor volumes were monitored for 28 days post tumor implantation.

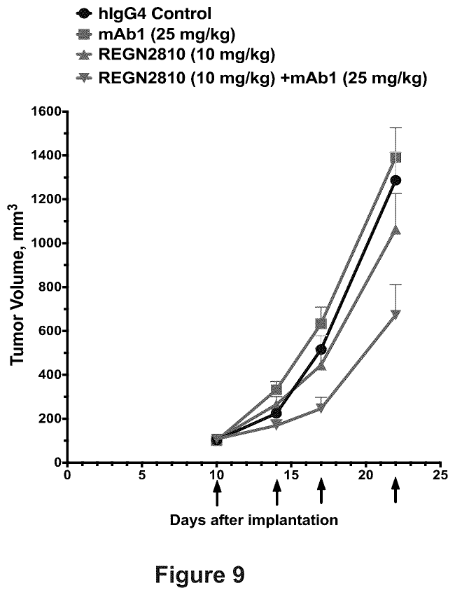

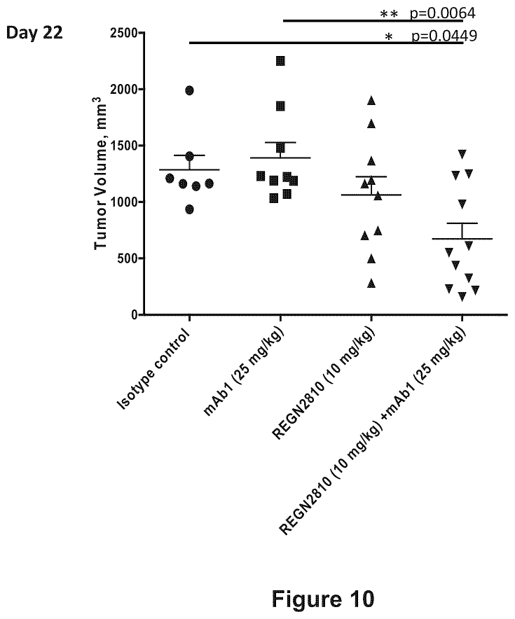

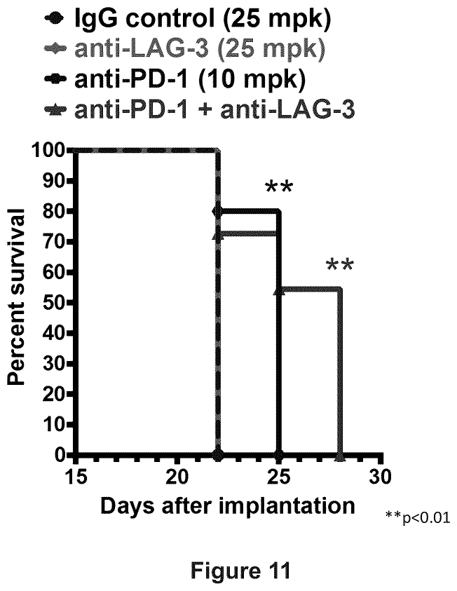

Treatment of MC38.ova tumor-bearing humanized mice with a combination of anti-hPD-1 and anti-hLAG-3 antibodies triggered activation of intratumoral and peripheral T cells. REGN2810 monotherapy resulted in partial tumor growth inhibition, while mAb1 monotherapy did not show reduction in mean tumor volume compared to isotype control treated mice (FIG. 9). By contrast, combination of mAb1 (25 mg/kg) and REGN2810 (10 mg/kg) demonstrated robust inhibition of MC38.Ova tumor growth. One-way analysis of variance (ANOVA) with Bonferroni's multiple comparison post-test revealed a significant difference in mean tumor volumes between mAb1 and REGN2810 combination group compared to mAb1 monotherapy (p<0.01) or isotype control (p<0.05) (FIG. 10). When combination therapy was compared to REGN2810 monotherapy, the overall mean tumor volumes over the course of the experiment were lower for combination therapy but this difference didn't reach statistical significance. The combination of anti-LAG-3 antibody and REGN2810 also resulted in significant increase in animal survival, as well as in duration of survival (p<0.01, as analyzed by log rank Mantel-Cox test). At day 25, 50% of mice treated with the combination therapy survived as compared to control and monotherapy (FIG. 11). Based on the results, the combination treatment exhibited an additive, dose dependent anti-tumor effect compared to the respective monotherapies.

### Example 16: Pharmacokinetics of an Anti-LAG-3 Antibody in Cynomolgus Monkey

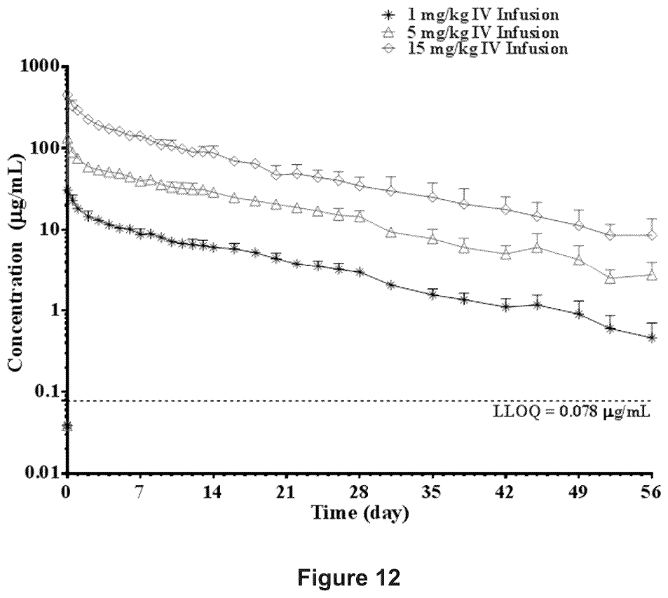

Pharmacokinetics (PK) of anti-LAG3 antibody mAb1 was characterized after a single intravenous (IV) dose of 1.0, 5.0, or 15.0 mg/kg to female cynomolgus monkeys (5 animals per dose group). Blood samples for the determination of functional mAb1 concentration were collected at various time points from all animals. Concentrations of functional mAb1 in serum were determined using an enzyme-linked immunosorbent assay (ELISA). Anti-mAb1 antibodies in serum were analyzed using an electrochemiluminescence-based bridging immunoassay. The PK parameters were estimated using non-compartmental analysis (NCA). Mean concentration-time profiles are characterized by an initial brief distribution phase, followed by a linear beta elimination phase (FIG. 12). No target-mediated clearance phase was observed over the 8 week study duration. Mean AUC_{inf}, increased in a dose-proportional manner as demonstrated by similar AUC_{inf},/Dose values across the tested dose levels. Consistent with this finding, the mean total body clearance (C_{L}) values were similar for the 3 dose groups. In addition, the beta phase half-lives (t_{1/2}) for the 3 dose groups were comparable, ranging from 10.8 to 11.5 days. Based on the results, no-observed-adverse-effect level (NOAEL) could be established up to 50 mg/kg. These results indicated that mAb1 demonstrated linear kinetics under the conditions of this study.

### Example 17: Assessment of Toxicology of Anti-LAG3 Antibody in Cynomolgus Monkeys

The in vivo toxicology and toxicokinetic profiles of mAb1 were evaluated during a 4-week, repeat-dose, GLP-compliant toxicology study in male and female cynomolgus monkeys. Monkeys (5 animals/sex/group) were given 0, 2, 10, or 50 mg/kg/week mAb1 by IV infusion. Assessment of toxicity was based on mortality, moribundity, clinical observations, body weights, food consumption, ophthalmic examinations, ECG evaluations, respiration rates, pulse oximetry, body temperature, and blood pressure evaluations, and neurologic examinations. Clinical pathology parameters (hematology, coagulation, clinical chemistry, and urinalysis) and immunophenotyping were also evaluated. Gross necropsy examinations, measurement of organ weights, and histopathologic evaluations were conducted. Complete necropsies were performed at the end of the dosing period or at the end of the 8-week recovery period. Selected organs were weighed, and tissues were examined macroscopically and microscopically.

mAb1 was well tolerated at all dose levels evaluated when administered via weekly IV infusion to cynomolgus monkeys. Because mAb1 was well tolerated and there were no mAb1-related changes in any of the safety parameters evaluated, the no-observed-adverse-effect-level (NOAEL) for the study is considered to be 50 mg/kg/dose, the highest dose administered in this study.

### Example 18: Hydrogen/Deuterium Exchange (H/D)-based Epitope Mapping of Anti-LAG3 Antibody H4sH15482P Binding to the Extracellular Domain of hLAG3

Experiments were conducted to determine the amino acid residues of the human LAG3 extracellular domain (amino acids Leu23-Leu450 of human LAG3, UniProt Accession Number P18627, produced with a human IgG1 tag at the c-terminus (“hLag3.Fc”) (R&D Systems, Minneapolis, MN) (SEQ ID NO: 587) with which the anti-LAG3 antibody H4sH15482P interacts. For this purpose, Hydrogen/Deuterium (H/D) Exchange epitope mapping with mass spectrometry (HDX-MS) was utilized. A general description of the HDX method is set forth in Ehring (1999) Analytical Biochemistry 267(2):252-259; and Engen and Smith (2001) Anal. Chem. 73:256A-265A.

### Experimental Procedure

HDX-MS experiments were performed on an integrated Waters HDX/MS platform (Waters Corporation, Milford, MA), consisting of a Leaptec HDX PAL system for the deuterium labeling, a Waters Acquity M-Class (Auxiliary solvent manager) for the sample digestion and loading, a Waters Acquity M-Class (pBinary solvent manager) for the analytical column gradient, and a Synapt G2-Si mass spectrometer for peptic peptide mass measurement.

The labeling solution was prepared in 10 mM PBS buffer in D₂O at pD 7.0. For deuterium labeling, 3.8 μL of hLAG3.Fc (5 pmol/μL) or hLAG3.Fc premixed with the anti-Lag3 antibody H4sH15482P in a 1:1 molar ratio was incubated with 56.2 μL D₂O labeling solution for various time-points (e.g., undeuterated control=0 sec, deuterium labeling: 1 min and 20 min). The deuteration was quenched by transferring 50 μL of sample to 50 μL of pre-chilled quench buffer (0.2 M TCEP, 6 M guanidine chloride in 100 mM phosphate buffer, pH 2.5) and the mixture was incubated at 1.0° C. for 4 min. The quenched sample was then injected into a Waters HDX Manager for online pepsin/protease XIII digestion. The digested peptides were trapped onto an ACQUITY UPLC BEH C18 1.7-μm, 2.1×5 mm VanGuard pre-column (Waters Corporation) at 0° C. and eluted to an analytical column ACQUITY UPLC BEH C18 1.7-μm, 1.0×50 mm for a 9-minute gradient separation of 5%-40% B (mobile phase A: 0.1% formic acid in water, mobile phase B: 0.1% formic acid in acetonitrile). The mass spectrometer was set at cone voltage of 37 V, a scan time of 0.5 s, and mass/charge range of 50-1700 Thomson units (Th).

For the identification of the peptide residues of human Lag3 with which H4sH15482P interacts, LC-MS^{E} data from the undeuterated sample were processed and searched against a database that included sequences for human LAG3, pepsin, and randomized sequences using the Waters ProteinLynx Global Server (PLGS) software. The identified peptides were imported to DynamX software and filtered by two criteria: 1) minimum products per amino acid=0.2, and 2) replication file threshold=3. DynamX software then automatically determined deuterium uptake of each peptide based on retention time and high mass accuracy (<10 ppm) across multiple time points with 3 replicates at each time.

### Results

Using the online pepsin/protease XIII column coupled with MS^{E} data acquisition, a total of 123 peptides from human LAG3 were reproducibly identified in the absence or presence of the antibody, representing 78.3% sequence coverage. Four Lag-3 peptides had significantly reduced deuteration uptake (centroid delta values >0.8 daltons with p-values <0.05) when bound to H4sH15482P. The recorded peptide mass corresponds to the average value of the centroid MH⁺ mass from three replicates. These peptides, corresponding to amino acids 34-77 of hLAG3.Fc, (SEQ ID NO: 587), had slower deuteration rates when bound to H4sH15482P. These identified residues also correspond to residues 28-71 of human LAG3 as defined by Uniprot entry P18627 (LAG3_HUMAN, SEQ ID NO: 588), and as illustrated in Table 18.

TABLE 18
Human LAG3 Peptides with Altered Deuteration Rates upon Binding H4sH15482P

| Amino acid | T = 1 min deuteration | | | T = 20 min deuteration | | |
|---|---|---|---|---|---|---|
| residues of | | hLAG3.Fc + | | | hLAG3.Fc + | |
| SEQ ID NO: | hLAG3.Fc; | H4sH15482P; | | hLAG3.Fc; | H4sH15482P; | |
| 588 | MH⁺ | MH⁺ | Δ | MH⁺ | MH⁺ | Δ |
| 28-69 | 4352.30 ± 0.03 | 4350.14 ± 0.06 | −2.15 | 4352.33 ± 0.20 | 4351.43 ± 0.12 | −0.90 |
| 28-71 | 4672.54 ± 0.03 | 4670.42 ± 0.01 | −2.12 | 4672.69 ± 0.21 | 4671.57 ± 0.18 | −1.13 |
| 31-52 | 2198.11 ± 0.10 | 2196.49 ± 0.01 | −1.61 | 2198.97 ± 0.18 | 2197.18 ± 0.10 | −1.79 |
| 32-69 | 3853.07 ± 0.12 | 3851.37 ± 0.07 | −1.69 | 3853.23 ± 0.07 | 3852.35 ± 0.14 | −0.88 |

### Example 19: Clinical Trial of Anti-LAG3 Antibody in Patients with Advanced Malignancies

This Example describes a clinical trial to assess the safety, tolerability and anti-tumor activity of an anti-LAG3 antibody as monotherapy and in combination with an anti-PD-1 antibody in patients with advanced malignancies.

The exemplary anti-LAG3 antibody used for this study is a fully human antibody that binds specifically to human LAG3 and comprises HCDR1-HCDR2-HCDR3-LCDR1-LCDR2-LCDR3 of SEQ ID NOs: 420-422-424-428-430-432 and HCVR/LCVR of SEQ ID Nos: 418/426 (also known as H4sH15482P and hereinafter “mAb1”).

The anti-PD-1 antibody used for this study is H4H7798N (as disclosed in US20150203579; also known as REGN2810). REGN2810 (Anti-hPD-1) binds with high affinity to human PD-1 and blocks PD-1 interaction with PD-L1 and PD-L2.

### Study Objectives

The primary objectives of the study are:

In the Dose Escalation Phase: To evaluate safety and pharmacokinetics (PK) in order to determine an phase 2 dose of mAb1 as monotherapy and in combination with REGN2810 in patients with advanced malignancies, including lymphoma. The endpoints include rate of dose limiting toxicities (DLTs), adverse events (AEs) (including immune-related), serious adverse events (SAEs), deaths, and laboratory abnormalities (grade 3 or higher per Common Terminology Criteria for Adverse Events [CTCAE]), and pharmacokinetics.

In the Dose Expansion Phase: To assess preliminary anti-tumor activity of mAb1 alone and in combination with REGN2810 (separately by cohort) as measured by ORR. The endpoints include overall response rate (ORR) based on Response Evaluation Criteria in Solid Tumors (RECIST) 1.1 (Eisenhauer et al 2009, Eur. J. Cancer 45: 228-247) (solid tumors) and Lugano criteria (Cheson et al 2014, J. Clin. Oncol. 32: 3059-3068) (lymphoma).

The secondary objectives are: (a) to assess preliminary anti-tumor activity of mAb1 alone and in combination with REGN2810 (separately by cohort) as measured by ORR based on immune-related Response Evaluation Criteria in Solid Tumors (irRECIST) (Wolchok et al 2009, Clin. Cancer Res. 15: 7412-7420; Nishino et al 2013, Clin. Cancer Res. 19: 3936-3943), best overall response (BOR), duration of response (DOR), disease control rate, and PFS based on RECIST, irRECIST, and Lugano criteria (Cheson et al 2014, J. Clin. Oncol. 32: 3059-3068); (b) to characterize the safety profile in each expansion cohort as determined in the dose escalation phase; (c) to characterize the PK of mAb1 as monotherapy and mAb1 and REGN2810 when given in combination; and (d) to assess immunogenicity as measured by anti-drug antibodies (ADA) for mAb1 and REGN2810

The exploratory objectives of the study are: (a) to assess tumor volume; (b) to explore the pharmacodynamic effects of REGN2810 in serum, plasma, peripheral blood mononuclear cells (PBMC), and in tumor tissue samples (including archival tumor tissue) obtained at baseline, during treatment, and at the time of progression from patients treated with mAb1 as monotherapy or in combination with REGN2810; and (c) to assess the predictive potential and correlation to clinical response for biomarkers of interest that may include, but are not limited to: (i) circulating tumor nucleic acids; (ii) PBMC subset distribution and expression of immune checkpoint molecules and other biomarkers of interest; (iii) tumor RNA expression; (iv) number and distribution of tumor infiltrating lymphocytes (CD8+ T cells, CD4+ T cells, T regulatory cells, and tissue permitting, other subtypes such as B cells, myeloid-derived cells, NK cells, etc.); (v) expression levels (messenger RNA and/or protein) of PD-1, PD-L1, LAG3, and possibly other checkpoint modulators; (vi) mutations in known oncogenes and potential tumor neo-antigens; and (vii) tumor mutational burden.

### Study Design

This is a phase 1, first-in-human (FIH), open-label, multicenter, dose-escalation study of the safety, tolerability, activity and PK of mAb1 administered as monotherapy and in combination with REGN2810 in patients with advanced malignancies. Three dose levels of mAb1 (1.0, 3.0 and 10 mg/kg) are investigated as monotherapy and in combination with REGN2810 at a dose of 3.0 mg/kg.

After a screening period of up to 28 days, patients receive up to seventeen 21-day treatment cycles (for a total of up to 51 weeks of treatment), followed by a 24-week follow-up period. Each patient receives mAb1 (+/− REGN2810) every 21 days.

Treatment continues until the 51-week treatment period is complete, or until disease progression, unacceptable toxicity, withdrawal of consent, or study withdrawal criterion is met. Response assessment is every 6 weeks for the first 24 weeks, then every 9 weeks for the subsequent 27 weeks, regardless of delays in dosing of study drugs. After a minimum of 24 weeks of treatment (minimum 8 treatment cycles), patients with confirmed complete remission (CR) may elect to discontinue treatment and continue with all relevant study assessments. Similarly, after consultation with the physician, patients who have been treated for a minimum of 24 weeks with stable disease (SD) or partial response (PR) that has been maintained for 3 successive tumor evaluations may elect to discontinue treatment but continue with all relevant study assessments. For patients who experience a response and subsequently progress, a tumor biopsy at the time of progression is requested. Patients who tolerate 4 doses of mAb1 monotherapy and who have an initial tumor assessment of at least SD, but who subsequently demonstrate progressive disease (PD), have the option of adding REGN2810 3.0 mg/kg to the highest dose of mAb1 safely administered up to that point (if one is known) in an attempt to “rescue” a response by combined lymphocyte activation gene 2 (LAG-3) and programmed death 1 (PD-1) blockade. Safety evaluations are conducted at each study drug dosing visit.

### Dose Escalation

Three dose escalation monotherapy and combination therapy cohorts are planned to be enrolled (1.0, 3.0 and 10 mg/kg mAb1 with or without 3 mg/kg REGN2810). The first cohort to be enrolled receives mAb1 monotherapy at 1.0 mg/kg. Subsequent enrollment of each additional cohorts may be limited by the number of dose-limiting toxicities (DLT) observed in prior cohorts. Dose de-escalation cohorts (0.3 mg/kg mAb1 with or without 3 mg/kg REGN2810) are enrolled if necessary.

### Dose Escalation Rules

A modification of the traditional 3+3 design (“4+3”) is used for evaluation of all dose levels for both mAb1 monotherapy and mAb1+ REGN2810 cohorts. Although a minimum of 3 patients at each dose level are required to be evaluable for DLT, to maximize the efficiency of the phase 1 dose escalation while maintaining patient safety, 4 patients are enrolled at each dose level in case a patient discontinues prior to being evaluable for DLT. The DLT evaluation period is 28 days. Tolerability of a dose level is only achieved if all potentially DLT evaluable patients complete the 28-day DLT period without a DLT (0/3 or 0/4). If 3 patients complete the DLT period without experiencing a DLT, but there is a fourth patient in the DLT evaluation period, tolerability of the dose level is only achieved when the fourth patient completes the DLT evaluation period or discontinues therapy prior to being evaluable for DLT. If there is 1 DLT out of either 3 or 4 DLT-evaluable patients, then 4 or 3 more patients (respectively) are enrolled for a total of 7 patients. Similarly, 1/6 or 1/7 DLTs are considered tolerable. Two DLTs out of 2 to 7 evaluable patients will have exceeded the maximum tolerated dose (MTD). At the highest dose level tolerated, to further evaluate safety, additional 3 to 4 patients will be enrolled for a total of 6 to 10 DLT-evaluable patients. Zero to 1 out of 6 to 8, or 2 out of 9 to 10 DLTs will be considered acceptable. To further evaluate safety, 3 additional patients may be enrolled at any dose level at the discretion of the sponsor (in consultation with investigators).

If 1.0 mg/kg mAb1 (dose level 1; DL1) is deemed safe (after 3 to 7 patients), enrollment will commence at 3.0 mg/kg mAb1 (DL2). Following enrollment of 4 patients in DL2, enrollment in the first combination cohort (1.0 mg/kg mAb1+3.0 mg/kg REGN2810; DL3) may begin. Dose escalation to 10 mg/kg mAb1 (DL4) may commence once 3.0 mg/kg mAb1 (DL2) is deemed safe. The second combination cohort (3.0 mg/kg mAb1+3.0 mg/kg REGN2810; DL5) only enrolls after both DL2 and DL3 are deemed safe. The third combination cohort (10 mg/kg mAb1+3.0 mg/kg REGN2810; DL6) only enrolls after both DL4 and DL5 are deemed safe. If multiple cohorts are open simultaneously, priority is given to the lower number cohort (ie, DL2 over DL3 or DL3 over DL4).

### Dose-Limiting Toxicities

The DLT observation period for determination of safety for dose escalation or initiation of new combination therapy is defined as 28 days starting with cycle 1, day 1, with the intent to monitor the safety and tolerability of the first 2 doses of study drug(s) (mAb1 with or without REGN2810 as applicable). To be evaluable for a DLT, a patient must have received at least the first 2 doses of study drug(s) (ie, day 1 and day 22) and be monitored for at least 28 days following the first administration, and at least 7 days from the second administration or experienced a DLT (defined below) prior to the completion of the DLT period. Delays in the administration of the second dose of study drug(s) beyond day 35 and/or study drug discontinuation are considered a DLT if study drug-related. The duration of the DLT observation period is therefore longer for patients whose second dose is delayed, and for patients experiencing an AE for which the duration must be assessed in order to determine if the event was a DLT.

In addition to the inability to administer (due to study drug toxicity) dose #2 within the window, a DLT in general is defined as any of the following study drug-related toxicities:

### Non-Hematologic Toxicity:

Grade >2 uveitis
Any grade >3 non-hematologic toxicity (excluding clinically insignificant laboratory abnormalities such as asymptomatic elevations in amylase or lipase)

Hematologic Toxicity:
Grade 4 neutropenia lasting >7 days
Grade 4 thrombocytopenia
Grade 3 thrombocytopenia with bleeding
Grade >3 febrile neutropenia or grade >3 neutropenia with documented infection

Both irAEs and non-irAEs that meet the definition of a DLT are considered to be DLTs.

### Maximum Tolerated Dose

The MTD is defined as the dose level immediately below that at which dosing is stopped due to 2 or more DLTs out of 6 to 7 evaluable patients, and will be determined separately for monotherapy and combination therapy. However, due to the known occurrence of AEs due to monotherapy with REGN2810 and other PD-1/PD-L1 antibodies, for the combination cohorts the intensity, frequency and novelty of combination toxicity may be considered in the determination of MTD and the decision to add additional patients at a dose level. If dose escalation is not stopped for DLTs, it will be considered that the MTD has not been determined. An additional 3 patients will enroll in each of the monotherapy and combination cohorts deemed the highest dose levels tolerated (ie, 6 to 10 patients in each of these cohorts). If dose escalation for mAb1 monotherapy or combination therapy with REGN2810 is stopped at 1.0 mg/kg due to DLTs, a cohort is enrolled at a dose of 0.3 mg/kg. If dose escalation for mAb1 monotherapy or combination therapy is stopped due to DLTs at the 3.0 or 10 mg/kg dose level, the dose of mAb1 is reduced to the previously tested dose level for newly enrolled patients (in monotherapy or combination therapy cohorts respectively). No patients are allowed to initiate combination therapy with a dose of mAb1 that was not tolerable as monotherapy.

### Recommended Phase 2 Dose

The RP2D for the expansion cohorts will be no higher than the MTD or highest dose tested and may be different for monotherapy and combination therapy cohorts. The determination of the RP2D will be based on safety and PK data.

Patients in monotherapy cohorts who tolerate 4 doses of mAb1 and who have an initial tumor assessment of at least SD, but who subsequently demonstrate PD, have the option of adding REGN2810 3.0 mg/kg to the highest dose of mAb1 safely administered up to that point (if one is known) in an attempt to “rescue” a response by combined LAG-3 and PD-1 blockade.

### Expansion Cohorts

Once tolerability of mAb1 has been established alone and in combination with REGN2810, multiple expansion cohorts using monotherapy or combination therapy in select indications [non-small cell lung cancer (NSCLC), clear cell renal cancer (ccRCC), triple-negative breast cancer (TNBC), melanoma, and diffuse large B cell lymphoma (DLBCL)] are enrolled to further evaluate safety and seek preliminary evidence of anti-tumor activity in tumors where LAG3 is known to be expressed or over-expressed; anti-tumor activity of mAb1 alone or in combination with REGN2810 may occur. Enrollment in the expansion cohorts begins after dose escalation is complete. An additional 10 expansion cohorts (Table 19) utilizing the Simon 2-stage design (Simon 1989, Controlled Clinical Trials 10: 1-10) are enrolled after confirmation of the RP2D. A patient is assigned to a specific treatment cohort based on the patient's tumor type, presence or absence of prior anti-PD-1/anti-programmed death ligand 1 (PD-L1) therapy, the investigator's assessment of the appropriateness of a therapy regimen for that patient, and the availability of patient slots in the assigned treatment cohort. If safety issues develop in an individual expansion cohort during stage 1 of the Simon 2-stage design, enrollment may be paused. After a discussion between the investigators and Regeneron, enrollment may be resumed at the same or lower dose of mAb1. Safety issues triggering a pause could include early or late safety events. Patients are treated with mAb1 (dose determined by dose escalation findings) monotherapy or combination of mAb1 and REGN2810 3 mg/kg Q3W for up to 51 weeks. The enrollment of stage 2 for each expansion cohort occurs only if the minimum number of tumor responses is observed at stage 1.

TABLE 19
Expansion Cohorts Table

| Expan- | | Anti-PD- | Anti-PD- | |
|---|---|---|---|---|
| sion | | 1/PD-L1 | 1/PD-L1 | |
| Cohort | Tumor Type | Naïve | experienced | Treatment |
| 1 | Non small-cell | X | | Combination mAb1 |
| | lung cancer | | | and REGN2810 |
| 2 | Non small-cell | | X | Combination mAb1 |
| | lung cancer | | | and REGN2810 |
| 3 | Clear cell | X | | Combination mAb1 |
| | renal cancer | | | and REGN2810 |
| 4 | Clear cell | | X | Combination mAb1 |
| | renal cancer | | | and REGN2810 |
| 5 | Triple negative | X | | Combination mAb1 |
| | breast cancer | | | and REGN2810 |
| 6 | Melanoma | X | | Combination mAb1 |
| | | | | and REGN2810 |
| 7 | Melanoma | | X | Combination mAb1 |
| | | | | and REGN2810 |
| 8 | Diffuse large B | X | | mAb1 monotherapy |
| | cell lymphoma | | | |
| 9 | Diffuse large B | X | | Combination mAb1 |
| | cell lymphoma | | | and REGN2810 |
| 10 | Diffuse large B | | X | Combination mAb1 |
| | cell lymphoma | | | and REGN2810 |

### Study Population

In the Dose Escalation Phase: Patients with advanced malignancies who have not received prior therapy with an anti-LAG3 drug and/or PD-1/PD-L1 inhibitor and who are not candidates for standard therapy, or for whom no available therapy is expected to convey clinical benefit, and patients with malignancies that are incurable and have failed to respond to or have shown tumor progression despite standard therapy.

In the Dose Expansion Cohorts: Patients with select malignancies who have not received prior therapy with an anti-LAG-3 drug and who:
have not previously received therapy with anti-PD-1/PD-L1 but are appropriate candidates to receive anti-PD-1-based therapy (cohorts 1, 3, 5, 6, and 9) or
have previously received anti-PD-1/PD-L1 based therapy and had a confirmed objective response (CR or PR) or SD for at least 3 months on anti-PD-1/PD-L1 therapy but subsequently progressed on that therapy or had SD or a PR as best response with subsequent stable response for 6 months (cohorts 2, 4, 7, and 10) or
are not candidates for standard therapy, or for whom no available therapy is expected to convey clinical benefit and are appropriate for mAb1 monotherapy (cohort 8)

Inclusion Criteria: A patient must meet the following criteria to be eligible for inclusion in the study:
1. Men and women >18 years of age.
2. Dose escalation cohorts: Patients with histologically or cytologically confirmed diagnosis of malignancy (including lymphoma) with demonstrated progression of a tumor for whom there is no alternative standard of care therapeutic option, or no alternative standard of care with curative potential (ie, failed to respond to or developed tumor progression despite standard therapy), AND have not been previously treated with a PD-1/PD-L1 inhibitor. These patients do not require measurable disease per RECIST 1.1 or Lugano criteria.
3. Dose expansion cohorts: Patients with histologically or cytologically confirmed diagnosis of 1 of the following tumors with measurable disease per RECIST 1.1 or Lugano criteria meeting the following criteria:
   i. Anti-PD-1/PD-L1 naïve stage IIIB or IV NSCLC either without prior therapy for metastatic disease or with disease progression/recurrence after one platinum-containing regimen (cohort 1)
   ii. Anti-PD-1/PD-L1 experienced* stage IllB or IV NSCLC with no more than 2 prior therapies for metastatic disease (cohort 2)
   iii. Anti-PD-1/PD-L1 naïve advanced or metastatic ccRCC with a clear cell component who had received 1 to 2 previous regimens of anti-angiogenic therapy (cohort 3)
   iv. Anti-PD-1/PD-L1 experienced* advanced or metastatic ccRCC with a clear cell component who had received 1 to 2 previous regimens of anti-angiogenic therapy (cohort 4)
   v. Anti-PD-1/PD-L1 naïve metastatic TNBC (estrogen, progesterone, and human epidermal growth factor receptor 2 negative) who have received 5 or fewer prior lines of therapy (cohort 5)
   vi. Anti-PD-1/PD-L1 naïve advanced or metastatic melanoma who have received no more than 2 previous regimens for metastatic disease (cohort 6)
   vii. Anti-PD-1/PD-L1 experienced* advanced or metastatic melanoma who have received no more than 2 previous regimens for metastatic disease (cohort 7)
   viii. Anti-PD-1/PD-L1 naïve relapsed/refractory DLBCL who have either progressed after or are not candidates for autologous stem cell transplant (cohorts 8 and 9)
   ix. Anti-PD-1/PD-L1 experienced* relapsed/refractory DLBCL who have either progressed after or are not candidates for autologous stem cell transplant (cohort 10)
NOTE: *Anti-PD-1/PD-L1 experienced is defined as having tolerated or tolerating anti-PD-1/PD-L1 therapy (ie, did not discontinue due to toxicity) and having had disease control with either:
a. a CR/PR confirmed by repeat imaging or SD for at least 12 weeks from first dose, prior to disease progression. Disease progression must have either been on therapy with anti-PD-1/PD-L1 or within 12 weeks of the last dose.
b. SD or a PR as best response with subsequent stable response (no greater than 70% decline from baseline) for 6 months while on anti-PD-1/PD-L1 therapy.
4. Eastern Cooperative Oncology Group performance status of 0 or 1
5. Life expectancy of at least 3 months
6. Adequate organ and bone marrow function as follows: (a) Hemoglobin >9.0 g/dL; (b) Absolute neutrophil count >1.5×10⁹/L; (c) Platelet count >75×10⁹/L; (d) Serum creatinine≤51.5× upper limit of normal (ULN) or estimated glomerular filtration rate >50 mL/min/1.73 m²; (e) Total bilirubin:1.5× ULN; (f) Aspartate aminotranseferase and alanine aminotransferase (ALT) 53× ULN or <5× ULN, if liver metastases; and/or (g) Alkaline phosphatase <2.5× ULN (or <5.0×ULN, if liver or bone metastases)
7. Willing and able to comply with clinic visits and study-related procedures
8. Provide signed informed consent.

Exclusion Criteria: A patient who meets any of the following criteria will be excluded from the study: 1. Currently receiving treatment in another study, or has participated in a study of an investigational agent and received treatment, or used an investigational device within 4 weeks of first dose of study therapy, or received treatment with an approved systemic therapy within 3 weeks of first dose of study therapy, or has received any previous systemic therapy within 5 half-lives of first dose of study therapy (whichever is longer). For expansion cohorts 2, 4, 7, 10 (anti-PD-1/PD-L1 experienced) only, prior anti-PD-1/PD-L1 therapy cannot have been given within 3 weeks of first dose of study therapy, regardless of half-life or approval status of the drug. 2. Prior treatment with any LAG-3 targeting biologic or small molecule 3. Radiation therapy within 2 weeks prior to randomization and not recovered to baseline from any AE due to radiation 4. Expansion cohorts only: Another malignancy that is progressing or requires active treatment with the exception of non-melanomatous skin cancer that has undergone potentially curative therapy or in situ cervical carcinoma, or any other tumor that has been deemed to be effectively treated with definitive local control for at least 2 years prior to enrollment. 5. Untreated or active central nervous system metastases. Patients with previously treated central nervous system metastases may participate provided they are stable (ie, without evidence of progression by imaging for at least 6 weeks prior to the first dose of study treatment, and any neurologic symptoms have returned to baseline), and there is no evidence of new or enlarging central nervous system metastases, and the patient does not require any systemic corticosteroids for management of central nervous system metastases within 4 weeks prior to the first dose of REGN2810. 6. Encephalitis, meningitis, or uncontrolled seizures in the year prior to informed consent 7. Ongoing or recent (within 5 years) evidence of significant autoimmune disease that required treatment with systemic immunosuppressive treatments, which may suggest risk for irAEs. The following are not exclusionary: vitiligo, childhood asthma that has resolved, hypothyroidism that required only hormone replacement, type 1 diabetes or psoriasis that does not require systemic treatment. 8. Corticosteroid therapy (>10 mg prednisone/day or equivalent) within 1 week prior to the first dose of study drug. Patients who require a brief course of steroids are not excluded. 9. Known history of, or any evidence of interstitial lung disease, or active, non-infectious pneumonitis (past 5 years) 10. Uncontrolled infection with human immunodeficiency virus, hepatitis B or hepatitis C infection; or diagnosis of immunodeficiency 11. Active infection requiring systemic therapy 12. Receipt of a live vaccine within 30 days of planned start of study medication 13. Major surgical procedure, open biopsy or significant traumatic injury within 4 weeks prior to screening 14. Myocardial infarction within 9 months prior to the first dose of study therapy 15. Prior allogeneic stem cell transplant 16. Any medical condition that in the opinion of the investigator would make participation in the study not in the best interest of the patient 17. Documented allergic or acute hypersensitivity reaction attributed to antibody treatments 18. Known allergy to doxycycline or other tetracycline antibiotics 19. Known psychiatric or substance abuse disorders that would interfere with participation with the requirements of the study 20. Sexually active men or women of childbearing potential unwilling to practice contraception during the study. Women who are pregnant, breastfeeding or expecting to conceive or men planning to father a child within the projected duration of the study (screening visit through 180 days after the last dose of study drug). 21. Sexually active men or women of childbearing potential who are unwilling to practice adequate contraception prior to the start of the first treatment, during the study, and for at least 6 months after the last dose of study drug is administered.

### Study Treatments

Monotherapy: mAb1 is administered in an outpatient setting by intravenous (IV) infusion over 30 minutes every 21 days for up to 51 weeks at the following monotherapy doses:
DL1: 1.0 mg/kg mAb1 IV infusion over 30 minutes every 21 days for 51 weeks
DL2: 3.0 mg/kg mAb1 IV infusion over 30 minutes every 21 days for 51 weeks
DL4: 10 mg/kg mAb1 IV infusion over 30 minutes every 21 days for 51 weeks
DL-1m: 0.3 mg/kg mAb1 IV infusion over 30 minutes every 21 days for 51 weeks (if necessary)

Combination Therapy: For combination therapy, the sequence of study drug administration is mAb1 first followed by REGN2810 on the same day. Study drugs are administered in an outpatient setting by IV infusion over 30 minutes each every 21 days for up to 51 weeks. Planned combination regimens to be assigned include:
DL3: 1.0 mg/kg mAb1 and 3.0 mg/kg REGN2810 IV infusion over 30 minutes each every 21 days for 51 weeks
DL5: 3.0 mg/kg mAb1 and 3.0 mg/kg REGN2810 IV infusion over 30 minutes each every 21 days for 51 weeks
DL6: 10.0 mg/kg mAb1 and 3.0 mg/kg REGN2810 IV infusion over 30 minutes each every 21 days for 51 weeks
DL-1c: 0.3 mg/kg mAb1 and 3.0 mg/kg REGN2810 IV infusion over 30 minutes each every 21 days for 51 weeks (if necessary)

### Study Endpoints

### Primary Endpoints

In the Dose Escalation Phase: Rate of DLTs, adverse events (AEs; including immune-related), serious adverse events (SAEs), deaths, and laboratory abnormalities (grade 3 or higher per Common Terminology Criteria for Adverse Events [CTCAE]), and PK

In the Dose Expansion Phase: Objective response rate (ORR) based on RECIST 1.1 (solid tumors) and Lugano criteria (lymphoma)

### Secondary Endpoints

ORR based on immune-related Response Evaluation Criteria in Solid Tumors (irRECIST); Best overall response (BOR), duration of response (DOR), disease control rate, and progression free survival (PFS) based on RECIST, irRECIST, and Lugano criteria); AEs; including immune-related, SAEs, deaths, and laboratory abnormalities (grade 3 or higher per CTCAE); PK and ADA

### Procedures and Assessments

The safety and tolerability of mAb1 alone or in combination with REGN2810 is monitored by clinical assessment of AEs and by repeated measurements of clinical evaluation including vital signs (temperature, blood pressure, pulse, and respiration), physical examinations (complete and limited), 12-lead electrocardiograms (ECGs), and laboratory assessment including standard hematology, chemistry and urinalysis.

Blood samples for the determination of functional mAb1 and functional REGN2810 in serum and ADA (anti-mAb1 or anti-REGN2810) samples arecollected. Serum and plasma samples are collected for analysis of additional biomarkers. Speculated pharmacodynamic, predictive and prognostic biomarkers related to mAb1 and REGN2810 treatment exposure, clinical activity, or underlying disease are investigated in serum, plasma, peripheral blood mononuclear cells (PBMCs), and tumor tissue. Anti-tumor activity is assessed by CT and MRI. A genomic DNA sample is collected from patients who have consented to the optional pharmacogenomics sub-study.

### Results

It is expected that mAb1 administration is safe and well tolerated by patients with advanced malignancies in the study. Combination with 3 mg/kg of REGN2810 is expected to lead to tumor regression as assessed by ORR in patients with solid tumors such as non-small-cell lung cancer, melanoma, clear cell renal cancer, B-cell lymphoma or breast cancer.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1-27. (canceled)

28. A method of inhibiting growth of a tumor or a tumor cell in a subject comprising administering to the subject in need thereof a therapeutically effective amount of an antibody or antigen-binding fragment thereof that binds specifically to human lymphocyte activation gene 3 (LAG3) protein, wherein the antibody or antigen-binding fragment thereof comprises three heavy chain complementarity determining regions (CDRs) (HCDR1, HCDR2 and HCDR3) contained within a heavy chain variable region (HCVR) amino acid sequence of SEQ ID NO: 418 and three light chain CDRs (LCDR1, LCDR2 and LCDR3) contained within a light chain variable region (LCVR) amino acid sequence of SEQ ID NO: 426,
and wherein the tumor is colon cancer or colorectal cancer.

29. The method of claim 28, wherein the tumor is a primary or a recurrent tumor.

30. The method of claim 28, wherein the tumor is an established tumor.

31. (canceled)

32. The method of claim 28, wherein the antibody or antigen-binding fragment thereof is administered as an initial dose followed by one or more secondary doses, wherein each secondary dose is administered 1 to 4 weeks after the immediately preceding dose.

33. The method of claim 32, wherein the antibody or antigen-binding fragment is administered at a dose of about 1, 3 or 10 mg/kg of body weight of the subject.

34. The method of claim 28, wherein the antibody or antigen-binding fragment thereof is administered to the subject in combination with a second therapeutic agent.

35. The method of claim 34, wherein the second therapeutic agent is selected from the group consisting of a CTLA inhibitor, an antibody to a tumor specific antigen, an antibody to a virally-infected-cell antigen, a PD-L1 inhibitor, a CD20 inhibitor, a bispecific antibody against CD20 and CD3, a dietary supplement such as an antioxidant, a VEGF antagonist, a chemotherapeutic agent, a cytotoxic agent, radiation, a NSAID, a corticosteroid, and any other therapy useful for ameliorating at least one symptom associated with the disease or disorder.

36. The method of claim 34, wherein the second therapeutic agent is a PD-1 inhibitor.

37. The method of claim 36, wherein the PD-1 inhibitor is REGN2810, nivolumab or pembrolizumab.

38. The method of claim 37, wherein the PD-1 inhibitor is administered at a dose of 1, 3 or 10 mg/kg of the subject's body weight.

39. The method of claim 28, wherein the antibody or antigen-binding fragment thereof is administered subcutaneously, intravenously, intradermally, intraperitoneally, orally, intramuscularly or intracranially.

40. A method for treating a disease or disorder that is treatable by antagonizing LAG3 comprising administering to a subject in need thereof a therapeutically effective amount of the antibody or antigen-binding fragment thereof that binds specifically to human lymphocyte activation gene 3 (LAG3) protein, wherein the antibody or antigen-binding fragment thereof comprises three heavy chain complementarity determining regions (CDRs) (HCDR1, HCDR2 and HCDR3) contained within a heavy chain variable region (HCVR) amino acid sequence of SEQ ID NO: 418 and three light chain CDRs (LCDR1, LCDR2 and LCDR3) contained within a light chain variable region (LCVR) amino acid sequence of SEQ ID NO: 426.

41. The method of claim 40, wherein the disease or disorder is a chronic viral infection caused by a virus selected from the group consisting of human immunodeficiency virus (HIV), hepatitis C virus (HCV), hepatitis B virus (HBV), human papilloma virus (HPV), lymphocytic choriomeningitis virus (LCMV) and simian immunodeficiency virus (SIV).

42. The method of claim 40, wherein the disease or disorder is colorectal cancer.

43. The method of claim 28, wherein the antibody or antigen-binding fragment thereof comprises a HCVR having the amino acid sequence of SEQ ID NO: 418.

44. The method of claim 28, wherein the antibody or antigen-binding fragment thereof comprises a LCVR having the amino acid sequence of SEQ ID NO: 426.

45. The method of claim 28, wherein the antibody or antigen-binding fragment thereof comprises HCDR1-HCDR2-HCDR3-LCDR1-LCDR2-LCDR3 comprising amino acid sequences of SEQ ID NOs: 420-422-424-428-430-432.

46. The method of claim 28, wherein the antibody or antigen-binding fragment thereof comprises a HCVR/LCVR amino acid sequence pair of SEQ ID NOs: 418/426.

47. The method of claim 28, wherein the antibody comprises a heavy chain and a light chain, wherein the heavy chain comprises the amino acid sequence of SEQ ID NO: 577.

48. The method of claim 28, wherein the antibody comprises a heavy chain and a light chain, wherein the light chain comprises the amino acid sequence of SEQ ID NO: 578.

49. The method of claim 28, wherein the antibody comprises a heavy chain/light chain amino acid sequence pair of SEQ ID NOs: 577/578.

50. The method of claim 40, wherein the antibody or antigen-binding fragment thereof comprises a HCVR having the amino acid sequence of SEQ ID NO: 418.

51. The method of claim 40, wherein the antibody or antigen-binding fragment thereof comprises a LCVR having the amino acid sequence of SEQ ID NO: 426.

52. The method of claim 40, wherein the antibody or antigen-binding fragment thereof comprises HCDR1-HCDR2-HCDR3-LCDR1-LCDR2-LCDR3 comprising amino acid sequences of SEQ ID NOs: 420-422-424-428-430-432.

53. The method of claim 40, wherein the antibody or antigen-binding fragment thereof comprises a HCVR/LCVR amino acid sequence pair of SEQ ID NOs: 418/426.

54. The method of claim 40, wherein the antibody comprises a heavy chain and a light chain, wherein the heavy chain comprises the amino acid sequence of SEQ ID NO: 577.

55. The method of claim 40, wherein the antibody comprises a heavy chain and a light chain, wherein the light chain comprises the amino acid sequence of SEQ ID NO: 578.

56. The method of claim 40, wherein the antibody comprises a heavy chain/light chain amino acid sequence pair of SEQ ID NOs: 577/578.


SEQUENCE LISTING (589 sequences)

SEQ ID NO: 1 - DNA, 372 bp, synthetic construct
  misc_feature 1..372: note = synthetic
  source 1..372: mol_type = other DNA
gaggtgcagctgttggagtctgggggaggcttggtacagcctggggggtccctgagactctcctgtgtggcctctggattcacctttagcacctatgccatgagttgggtccgccaggctccagggatggggctggagtgggtctcaagtattagtggtagtggtcgtaacacatactatgcagactccgtgaagggccggttcaccatctccagagacaattccaagaacacgctgtttcttcaaatgaacagcctgagagccgaggacacggccgtttattactgtgcgaaagagtccgtaactggaacttcgtcctactactacggtgtggacgtctggggccaagggaccacggtcaccgtctcctcg

SEQ ID NO: 2 - protein, 124 aa, synthetic construct
  REGION 1..124: note = synthetic
EVQLLESGGGLVQPGGSLRLSCVASGFTFSTYAMSWVRQAPGMGLEWVSSISGSGRNTYYADSVKGRFTISRDNSKNTLFLQMNSLRAEDTAVYYCAKESVTGTSSYYYGVDVWGQGTTVTVSS

SEQ ID NO: 3 - DNA, 24 bp, synthetic construct
  misc_feature 1..24: note = synthetic
  source 1..24: mol_type = other DNA
ggattcacctttagcacctatgcc

SEQ ID NO: 4 - protein, 8 aa, synthetic construct
  REGION 1..8: note = synthetic
GFTFSTYA

SEQ ID NO: 5 - DNA, 24 bp, synthetic construct
  misc_feature 1..24: note = synthetic
  source 1..24: mol_type = other DNA
attagtggtagtggtcgtaacaca

SEQ ID NO: 6 - protein, 8 aa, synthetic construct
  REGION 1..8: note = synthetic
ISGSGRNT

SEQ ID NO: 7 - DNA, 51 bp, synthetic construct
  misc_feature 1..51: note = synthetic
  source 1..51: mol_type = other DNA
gcgaaagagtccgtaactggaacttcgtcctactactacggtgtggacgtc

SEQ ID NO: 8 - protein, 17 aa, synthetic construct
  REGION 1..17: note = synthetic
AKESVTGTSSYYYGVDV

SEQ ID NO: 9 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagagcattagcagttatttaaattggtatcatcagaaaccagggaaagccccaaagctcctgatctatgctgcatccagtttgcaaaatggggtcccatcaaggttcagtggcagtggatctgggacagatttcactctcaccatcagcagtctgcaacctgaagattttgcatcttactactgtcaacagagttacagaaccccgctcactttcggcggagggaccaaggtggagatcaaa

SEQ ID NO: 10 - protein, 107 aa, synthetic construct
  REGION 1..107: note = synthetic
DIQMTQSPSSLSASVGDRVTITCRASQSISSYLNWYHQKPGKAPKLLIYAASSLQNGVPSRFSGSGSGTDFTLTISSLQPEDFASYYCQQSYRTPLTFGGGTKVEIK

SEQ ID NO: 11 - DNA, 18 bp, synthetic construct
  misc_feature 1..18: note = synthetic
  source 1..18: mol_type = other DNA
cagagcattagcagttat

SEQ ID NO: 12 - protein, 6 aa, synthetic construct
  REGION 1..6: note = synthetic
QSISSY

SEQ ID NO: 13-14 - skipped (2 entries)

SEQ ID NO: 15 - DNA, 27 bp, synthetic construct
  misc_feature 1..27: note = synthetic
  source 1..27: mol_type = other DNA
caacagagttacagaaccccgctcact

SEQ ID NO: 16 - protein, 9 aa, synthetic construct
  REGION 1..9: note = synthetic
QQSYRTPLT

SEQ ID NO: 17 - DNA, 387 bp, synthetic construct
  misc_feature 1..387: note = synthetic
  source 1..387: mol_type = other DNA
caggtgcagctggaggagtctgggggaggcgtggtccagcctgggaggtccctgagactctcctgtgcagcgtctggattcaccttcagttggtatggcatgcactgggtccgccaggctccaggcaaggggctggagtgggtggcacttatatggtatgatggaactaataaaaagtatggagactccgtgaagggccgattcaccatttccagagacaattccaagaacacggtgtatctgcaaatgaacagcctgagagccgaggacacggctgtgtattactgtgcgagagattgtggacatagtggcaacgatcgggggacttactattactactacggtatggacgtctggggccaagggaccacggtcaccgtctcctca

SEQ ID NO: 18 - protein, 129 aa, synthetic construct
  REGION 1..129: note = synthetic
QVQLEESGGGVVQPGRSLRLSCAASGFTFSWYGMHWVRQAPGKGLEWVALIWYDGTNKKYGDSVKGRFTISRDNSKNTVYLQMNSLRAEDTAVYYCARDCGHSGNDRGTYYYYYGMDVWGQGTTVTVSS

SEQ ID NO: 19 - DNA, 24 bp, synthetic construct
  misc_feature 1..24: note = synthetic
  source 1..24: mol_type = other DNA
ggattcaccttcagttggtatggc

SEQ ID NO: 20 - protein, 8 aa, synthetic construct
  REGION 1..8: note = synthetic
GFTFSWYG

SEQ ID NO: 21 - DNA, 24 bp, synthetic construct
  misc_feature 1..24: note = synthetic
  source 1..24: mol_type = other DNA
atatggtatgatggaactaataaa

SEQ ID NO: 22 - protein, 8 aa, synthetic construct
  REGION 1..8: note = synthetic
IWYDGTNK

SEQ ID NO: 23 - DNA, 66 bp, synthetic construct
  misc_feature 1..66: note = synthetic
  source 1..66: mol_type = other DNA
gcgagagattgtggacatagtggcaacgatcgggggacttactattactactacggtatggacgtc

SEQ ID NO: 24 - protein, 22 aa, synthetic construct
  REGION 1..22: note = synthetic
ARDCGHSGNDRGTYYYYYGMDV

SEQ ID NO: 25 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 26 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 27 - DNA, 18 bp, synthetic construct
  misc_feature 1..18: note = synthetic
  source 1..18: mol_type = other DNA
cagagcattagcagctat

SEQ ID NO: 28 - protein, 6 aa, synthetic construct
  REGION 1..6: note = synthetic
QSISSY

SEQ ID NO: 29-30 - skipped (2 entries)

SEQ ID NO: 31 - DNA, 30 bp, synthetic construct
  misc_feature 1..30: note = synthetic
  source 1..30: mol_type = other DNA
caacagagttacagtacccctccgatcacc

SEQ ID NO: 32 - protein, 10 aa, synthetic construct
  REGION 1..10: note = synthetic
QQSYSTPPIT

SEQ ID NO: 33 - DNA, 357 bp, synthetic construct
  misc_feature 1..357: note = synthetic
  source 1..357: mol_type = other DNA
caggtgcagctacagcagtggggcgcaggactgttgaagccttcggagaccctgtccctcacctgcgctgtctatggtgggtccttcagtggttactactggaactggatccgccagcccccagggaaggggctggagtgggttggggaaatcagtcatagaggaaccaccaactacaacccgtccctcaagagtcgagtcaccatatcactggacacgtccaagaaccagttctccctgaaactgacctctgtgaccgccgcggacacggctgtgtattactgttcgagagacgaggaactggaattccgtttctttgactactggggccagggaaccctggtcaccgtctcctca

SEQ ID NO: 34 - protein, 119 aa, synthetic construct
  REGION 1..119: note = synthetic
QVQLQQWGAGLLKPSETLSLTCAVYGGSFSGYYWNWIRQPPGKGLEWVGEISHRGTTNYNPSLKSRVTISLDTSKNQFSLKLTSVTAADTAVYYCSRDEELEFRFFDYWGQGTLVTVSS

SEQ ID NO: 35 - DNA, 24 bp, synthetic construct
  misc_feature 1..24: note = synthetic
  source 1..24: mol_type = other DNA
ggtgggtccttcagtggttactac

SEQ ID NO: 36 - protein, 8 aa, synthetic construct
  REGION 1..8: note = synthetic
GGSFSGYY

SEQ ID NO: 37 - DNA, 21 bp, synthetic construct
  misc_feature 1..21: note = synthetic
  source 1..21: mol_type = other DNA
atcagtcatagaggaaccacc

SEQ ID NO: 38 - protein, 7 aa, synthetic construct
  REGION 1..7: note = synthetic
ISHRGTT

SEQ ID NO: 39 - DNA, 39 bp, synthetic construct
  misc_feature 1..39: note = synthetic
  source 1..39: mol_type = other DNA
tcgagagacgaggaactggaattccgtttctttgactac

SEQ ID NO: 40 - protein, 13 aa, synthetic construct
  REGION 1..13: note = synthetic
SRDEELEFRFFDY

SEQ ID NO: 41 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcagagtgttagcagctatttagcctggtaccaacaaaaacctggccaggctcccaggctcctcgtctatggtgcatccaacagggccactggcatcccagccaggttcagtggcagtgggtctgggacagacttcactctcaccatcagcagcctagagcctgaagattttgcattttattactgtcagcagcgtagcaactggccgctcactttcggcggagggaccaaggtggagatcaaa

SEQ ID NO: 42 - protein, 107 aa, synthetic construct
  REGION 1..107: note = synthetic
EIVLTQSPATLSLSPGERATLSCRASQSVSSYLAWYQQKPGQAPRLLVYGASNRATGIPARFSGSGSGTDFTLTISSLEPEDFAFYYCQQRSNWPLTFGGGTKVEIK

SEQ ID NO: 43 - DNA, 18 bp, synthetic construct
  misc_feature 1..18: note = synthetic
  source 1..18: mol_type = other DNA
cagagtgttagcagctat

SEQ ID NO: 44 - protein, 6 aa, synthetic construct
  REGION 1..6: note = synthetic
QSVSSY

SEQ ID NO: 45-46 - skipped (2 entries)

SEQ ID NO: 47 - DNA, 27 bp, synthetic construct
  misc_feature 1..27: note = synthetic
  source 1..27: mol_type = other DNA
cagcagcgtagcaactggccgctcact

SEQ ID NO: 48 - protein, 9 aa, synthetic construct
  REGION 1..9: note = synthetic
QQRSNWPLT

SEQ ID NO: 49 - DNA, 357 bp, synthetic construct
  misc_feature 1..357: note = synthetic
  source 1..357: mol_type = other DNA
cagctgcagctgcaggagtcgggcccaggactggtgaagccttcggagaccctgtccctcacctgcactgtctctggtgactccatcatcagtaatagttattactggggctggatccgccagcccccagggaaggggctggagtggattggcaatttcttttatactggggccacctactacaacccgtccctcaagagtcgagtcaccatatccgctgacacgtccaagaatcagttctccctgaagctgagctctgtgaccgccgcagacacggctctgtattattgtgcgagttataataggaattaccggttcgacccctggggccagggaaccctggtcaccgtctcctca

SEQ ID NO: 50 - protein, 119 aa, synthetic construct
  REGION 1..119: note = synthetic
QLQLQESGPGLVKPSETLSLTCTVSGDSIISNSYYWGWIRQPPGKGLEWIGNFFYTGATYYNPSLKSRVTISADTSKNQFSLKLSSVTAADTALYYCASYNRNYRFDPWGQGTLVTVSS

SEQ ID NO: 51 - DNA, 30 bp, synthetic construct
  misc_feature 1..30: note = synthetic
  source 1..30: mol_type = other DNA
ggtgactccatcatcagtaatagttattac

SEQ ID NO: 52 - protein, 10 aa, synthetic construct
  REGION 1..10: note = synthetic
GDSIISNSYY

SEQ ID NO: 53 - DNA, 21 bp, synthetic construct
  misc_feature 1..21: note = synthetic
  source 1..21: mol_type = other DNA
ttcttttatactggggccacc

SEQ ID NO: 54 - protein, 7 aa, synthetic construct
  REGION 1..7: note = synthetic
FFYTGAT

SEQ ID NO: 55 - DNA, 33 bp, synthetic construct
  misc_feature 1..33: note = synthetic
  source 1..33: mol_type = other DNA
gcgagttataataggaattaccggttcgacccc

SEQ ID NO: 56 - protein, 11 aa, synthetic construct
  REGION 1..11: note = synthetic
ASYNRNYRFDP

SEQ ID NO: 57 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttacttctgtcaacagagttacagtacccctccgatcaccttcggccaagggacacgactggagattaaa

SEQ ID NO: 58 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYFCQQSYSTPPITFGQGTRLEIK

SEQ ID NO: 59 - DNA, 18 bp, synthetic construct
  misc_feature 1..18: note = synthetic
  source 1..18: mol_type = other DNA
cagagcattagcagctat

SEQ ID NO: 60 - protein, 6 aa, synthetic construct
  REGION 1..6: note = synthetic
QSISSY

SEQ ID NO: 61-62 - skipped (2 entries)

SEQ ID NO: 63 - DNA, 30 bp, synthetic construct; identical to SEQ ID NO: 31
  misc_feature 1..30: note = synthetic
  source 1..30: mol_type = other DNA

SEQ ID NO: 64 - protein, 10 aa, synthetic construct
  REGION 1..10: note = synthetic
QQSYSTPPIT

SEQ ID NO: 65 - DNA, 381 bp, synthetic construct
  misc_feature 1..381: note = synthetic
  source 1..381: mol_type = other DNA
caggtgcagctacagcagtggggcgcaggactgttgaagccttcggagaccctgtccctcacctgcgctgtctatggtgggtccttcagtacttactactggagctggatccgccagcccccagggaaggggctggagtggattggagagatcaatcatagtggaaacgccgactacaacccgtccctcaagagtcgagtctccatatcagtggacacgtccaagaaccagttctccctgaggctgagctctgtgaccgccgcggacacggctatttattactgtgcgagagcgggctattgtagtagtcccacctgctattcctactactacttcggtatggacgtctggggccaagggaccacggtcaccgtctcctca

SEQ ID NO: 66 - protein, 127 aa, synthetic construct
  REGION 1..127: note = synthetic
QVQLQQWGAGLLKPSETLSLTCAVYGGSFSTYYWSWIRQPPGKGLEWIGEINHSGNADYNPSLKSRVSISVDTSKNQFSLRLSSVTAADTAIYYCARAGYCSSPTCYSYYYFGMDVWGQGTTVTVSS

SEQ ID NO: 67 - DNA, 24 bp, synthetic construct
  misc_feature 1..24: note = synthetic
  source 1..24: mol_type = other DNA
ggtgggtccttcagtacttactac

SEQ ID NO: 68 - protein, 8 aa, synthetic construct
  REGION 1..8: note = synthetic
GGSFSTYY

SEQ ID NO: 69 - DNA, 21 bp, synthetic construct
  misc_feature 1..21: note = synthetic
  source 1..21: mol_type = other DNA
atcaatcatagtggaaacgcc

SEQ ID NO: 70 - protein, 7 aa, synthetic construct
  REGION 1..7: note = synthetic
INHSGNA

SEQ ID NO: 71 - DNA, 63 bp, synthetic construct
  misc_feature 1..63: note = synthetic
  source 1..63: mol_type = other DNA
gcgagagcgggctattgtagtagtcccacctgctattcctactactacttcggtatggacgtc

SEQ ID NO: 72 - protein, 21 aa, synthetic construct
  REGION 1..21: note = synthetic
ARAGYCSSPTCYSYYYFGMDV

SEQ ID NO: 73 - DNA, 324 bp, synthetic construct
  misc_feature 1..324: note = synthetic
  source 1..324: mol_type = other DNA
gaaattgtgttgacgcagtctccaggcaccctgtctttgtctctaggggaaagagccaccctctcctgcagggccagtcagagtgttatcagcagcttcttagcctggtaccagcagaaacctggccaggctcccaggctcctcatctatggtgcatccagcagggccactggcttcccagacaggttcagtggcagtgggtctgggacagacttcactctcaccatccgcagactggagcctgaagattttgcagtgtattactgtcagcagtatggtaactcaccttggacgttcggccaagggaccaaggtggagatcaaa

SEQ ID NO: 74 - protein, 108 aa, synthetic construct
  REGION 1..108: note = synthetic
EIVLTQSPGTLSLSLGERATLSCRASQSVISSFLAWYQQKPGQAPRLLIYGASSRATGFPDRFSGSGSGTDFTLTIRRLEPEDFAVYYCQQYGNSPWTFGQGTKVEIK

SEQ ID NO: 75 - DNA, 21 bp, synthetic construct
  misc_feature 1..21: note = synthetic
  source 1..21: mol_type = other DNA
cagagtgttatcagcagcttc

SEQ ID NO: 76 - protein, 7 aa, synthetic construct
  REGION 1..7: note = synthetic
QSVISSF

SEQ ID NO: 77-78 - skipped (2 entries)

SEQ ID NO: 79 - DNA, 27 bp, synthetic construct
  misc_feature 1..27: note = synthetic
  source 1..27: mol_type = other DNA
cagcagtatggtaactcaccttggacg

SEQ ID NO: 80 - protein, 9 aa, synthetic construct
  REGION 1..9: note = synthetic
QQYGNSPWT

SEQ ID NO: 81 - DNA, 387 bp, synthetic construct
  misc_feature 1..387: note = synthetic
  source 1..387: mol_type = other DNA
caggtcaccttgaaggagtctggtcctgtgctggtgaaacccacagagaccctcacgctgacctgcaccgtctctgggttctcactcagcaatgctgggatgggtgtgagctgggtccgtcagccccctgggaaggccctggagtggcttgcacacattttttcgaatgacgagaagtcctacagcacatctctgaggaccagactcaccatctccaaggacacctccaaaagccaggtggtccttaccgtgaccaacttggaccctgtggacacagccacatatttctgtgcacggataccagagtttaccagctcgtcgtgggctctctactacttctacggtatggacgtctggggccaagggaccacggtcaccgtctcctca

SEQ ID NO: 82 - protein, 129 aa, synthetic construct
  REGION 1..129: note = synthetic
QVTLKESGPVLVKPTETLTLTCTVSGFSLSNAGMGVSWVRQPPGKALEWLAHIFSNDEKSYSTSLRTRLTISKDTSKSQVVLTVTNLDPVDTATYFCARIPEFTSSSWALYYFYGMDVWGQGTTVTVSS

SEQ ID NO: 83 - DNA, 30 bp, synthetic construct
  misc_feature 1..30: note = synthetic
  source 1..30: mol_type = other DNA
gggttctcactcagcaatgctgggatgggt

SEQ ID NO: 84 - protein, 10 aa, synthetic construct
  REGION 1..10: note = synthetic
GFSLSNAGMG

SEQ ID NO: 85 - DNA, 21 bp, synthetic construct
  misc_feature 1..21: note = synthetic
  source 1..21: mol_type = other DNA
attttttcgaatgacgagaag

SEQ ID NO: 86 - protein, 7 aa, synthetic construct
  REGION 1..7: note = synthetic
IFSNDEK

SEQ ID NO: 87 - DNA, 63 bp, synthetic construct
  misc_feature 1..63: note = synthetic
  source 1..63: mol_type = other DNA
gcacggataccagagtttaccagctcgtcgtgggctctctactacttctacggtatggacgtc

SEQ ID NO: 88 - protein, 21 aa, synthetic construct
  REGION 1..21: note = synthetic
ARIPEFTSSSWALYYFYGMDV

SEQ ID NO: 89 - DNA, 324 bp, synthetic construct
  misc_feature 1..324: note = synthetic
  source 1..324: mol_type = other DNA
gaaattgtgttgacgcagtctccaggcaccctgtctttgtctccaggggaaagcgccaccctctcctgcagggccagtcagagtattaccagcacctacttcgcctggtaccagcagaaacctggccaggctcccaggctcctcatctatgctacatccagcagggccactggcgtcccagacaggttcagtggcagtgggtctgggacggacttcactctcaccatcagcagactggagcctgatgattttgcagtgtattactgtcagcaatatggtaggtcaccttggacgttcggccaagggaccaaggtggaagtcaaa

SEQ ID NO: 90 - protein, 108 aa, synthetic construct
  REGION 1..108: note = synthetic
EIVLTQSPGTLSLSPGESATLSCRASQSITSTYFAWYQQKPGQAPRLLIYATSSRATGVPDRFSGSGSGTDFTLTISRLEPDDFAVYYCQQYGRSPWTFGQGTKVEVK

SEQ ID NO: 91 - DNA, 21 bp, synthetic construct
  misc_feature 1..21: note = synthetic
  source 1..21: mol_type = other DNA
cagagtattaccagcacctac

SEQ ID NO: 92 - protein, 7 aa, synthetic construct
  REGION 1..7: note = synthetic
QSITSTY

SEQ ID NO: 93-94 - skipped (2 entries)

SEQ ID NO: 95 - DNA, 27 bp, synthetic construct
  misc_feature 1..27: note = synthetic
  source 1..27: mol_type = other DNA
cagcaatatggtaggtcaccttggacg

SEQ ID NO: 96 - protein, 9 aa, synthetic construct
  REGION 1..9: note = synthetic
QQYGRSPWT

SEQ ID NO: 97 - DNA, 363 bp, synthetic construct
  misc_feature 1..363: note = synthetic
  source 1..363: mol_type = other DNA
caggttcagctggtgcagtctggagctgaggtgaagaagcctggggcctcagtgaaggtctcctgcaaggcttctggttacacctttaccagttatggtatcagctgggtgcgacaggcccctggacaagggcttgagtggatgggatggatcagcgcttacaatgataacacaaactatgcacagaagctccagggcagagtcaccatgaccgcagacacatccacgaatacagcctacatggagctaaggagcctgagatctgacgacacggccatttattactgtgtgcgatggaattggggttccgtctactggtacttcgatctctggggccgtggcaccctggtcactgtctcctca

SEQ ID NO: 98 - protein, 121 aa, synthetic construct
  REGION 1..121: note = synthetic
QVQLVQSGAEVKKPGASVKVSCKASGYTFTSYGISWVRQAPGQGLEWMGWISAYNDNTNYAQKLQGRVTMTADTSTNTAYMELRSLRSDDTAIYYCVRWNWGSVYWYFDLWGRGTLVTVSS

SEQ ID NO: 99 - DNA, 24 bp, synthetic construct
  misc_feature 1..24: note = synthetic
  source 1..24: mol_type = other DNA
ggttacacctttaccagttatggt

SEQ ID NO: 100 - protein, 8 aa, synthetic construct
  REGION 1..8: note = synthetic
GYTFTSYG

SEQ ID NO: 101 - DNA, 24 bp, synthetic construct
  misc_feature 1..24: note = synthetic
  source 1..24: mol_type = other DNA
atcagcgcttacaatgataacaca

SEQ ID NO: 102 - protein, 8 aa, synthetic construct
  REGION 1..8: note = synthetic
ISAYNDNT

SEQ ID NO: 103 - DNA, 42 bp, synthetic construct
  misc_feature 1..42: note = synthetic
  source 1..42: mol_type = other DNA
gtgcgatggaattggggttccgtctactggtacttcgatctc

SEQ ID NO: 104 - protein, 14 aa, synthetic construct
  REGION 1..14: note = synthetic
VRWNWGSVYWYFDL

SEQ ID NO: 105 - DNA, 324 bp, synthetic construct
  misc_feature 1..324: note = synthetic
  source 1..324: mol_type = other DNA
gaaattgtgttgacgcagtctccaggcaccctgtctttgtctccaggggaaagagccaccctctcctgcagggccagtcagattattagcagcagctactttgcctggtaccagcagaaacctggccaggctcccaggctcctcatctatggtgcgtccagcagggccactggcatcccagacaggttcagtggcagtgtgtctgggacagacttcactctcaccatcagcagactggagcctgaagattttgcaatgtatttctgtcagcagtatggtaactcaccttggacgttcggccaagggaccaaggtggaaatcaaa

SEQ ID NO: 106 - protein, 108 aa, synthetic construct
  REGION 1..108: note = synthetic
EIVLTQSPGTLSLSPGERATLSCRASQIISSSYFAWYQQKPGQAPRLLIYGASSRATGIPDRFSGSVSGTDFTLTISRLEPEDFAMYFCQQYGNSPWTFGQGTKVEIK

SEQ ID NO: 107 - DNA, 21 bp, synthetic construct
  misc_feature 1..21: note = synthetic
  source 1..21: mol_type = other DNA
cagattattagcagcagctac

SEQ ID NO: 108 - protein, 7 aa, synthetic construct
  REGION 1..7: note = synthetic
QIISSSY

SEQ ID NO: 109-110 - skipped (2 entries)

SEQ ID NO: 111 - DNA, 27 bp, synthetic construct
  misc_feature 1..27: note = synthetic
  source 1..27: mol_type = other DNA
cagcagtatggtaactcaccttggacg

SEQ ID NO: 112 - protein, 9 aa, synthetic construct
  REGION 1..9: note = synthetic
QQYGNSPWT

SEQ ID NO: 113 - DNA, 351 bp, synthetic construct
  misc_feature 1..351: note = synthetic
  source 1..351: mol_type = other DNA
cagatcaccttgaaggagtctggtcctacgctggtgaaacccacacagaccctcacgctgacttgcaccttctctgggttctcactcaacactcatagagtgggtgtaggctggatccggcagcccccaggaaaggccctggagtggcttgcactcatttatgggaatgatgttaagaactacagcccatctctggagaccaggctcaccatcgccaaggacacctccaaaaaccaggtggtccttacaatgaccaacatggaccctgtggacacagccacatatttctgttcgtacataacgggggaaggaatgtactggggccagggaaccctggtcaccgtctcctca

SEQ ID NO: 114 - protein, 117 aa, synthetic construct
  REGION 1..117: note = synthetic
QITLKESGPTLVKPTQTLTLTCTFSGFSLNTHRVGVGWIRQPPGKALEWLALIYGNDVKNYSPSLETRLTIAKDTSKNQVVLTMTNMDPVDTATYFCSYITGEGMYWGQGTLVTVSS

SEQ ID NO: 115 - DNA, 30 bp, synthetic construct
  misc_feature 1..30: note = synthetic
  source 1..30: mol_type = other DNA
gggttctcactcaacactcatagagtgggt

SEQ ID NO: 116 - protein, 10 aa, synthetic construct
  REGION 1..10: note = synthetic
GFSLNTHRVG

SEQ ID NO: 117 - DNA, 21 bp, synthetic construct
  misc_feature 1..21: note = synthetic
  source 1..21: mol_type = other DNA
atttatgggaatgatgttaag

SEQ ID NO: 118 - protein, 7 aa, synthetic construct
  REGION 1..7: note = synthetic
IYGNDVK

SEQ ID NO: 119 - DNA, 27 bp, synthetic construct
  misc_feature 1..27: note = synthetic
  source 1..27: mol_type = other DNA
tcgtacataacgggggaaggaatgtac

SEQ ID NO: 120 - protein, 9 aa, synthetic construct
  REGION 1..9: note = synthetic
SYITGEGMY

SEQ ID NO: 121 - DNA, 333 bp, synthetic construct
  misc_feature 1..333: note = synthetic
  source 1..333: mol_type = other DNA
gatgttgtgatgactcagtctccactctccctgtccgtcacccttggacagccggcctccatttcctgtaggtctagtcaaaacctcatgtacagtgatggaaacacctacttgaattggtttcaccagaggccaggccaatctccaaggcgtctaatttataaggtttctaaccgggactctggggtcccagacagattcagcggcagtgggtcaggcactgatttcacactgaaaatcagcagggtggaggctgaggatgttggggtttattactgcatgcaaggtacacactggtacacatttggccaggggaccaagctggagatcaaa

SEQ ID NO: 122 - protein, 111 aa, synthetic construct
  REGION 1..111: note = synthetic
DVVMTQSPLSLSVTLGQPASISCRSSQNLMYSDGNTYLNWFHQRPGQSPRRLIYKVSNRDSGVPDRFSGSGSGTDFTLKISRVEAEDVGVYYCMQGTHWYTFGQGTKLEIK

SEQ ID NO: 123 - DNA, 33 bp, synthetic construct
  misc_feature 1..33: note = synthetic
  source 1..33: mol_type = other DNA
caaaacctcatgtacagtgatggaaacacctac

SEQ ID NO: 124 - protein, 11 aa, synthetic construct
  REGION 1..11: note = synthetic
QNLMYSDGNTY

SEQ ID NO: 125-126 - skipped (2 entries)

SEQ ID NO: 127 - DNA, 24 bp, synthetic construct
  misc_feature 1..24: note = synthetic
  source 1..24: mol_type = other DNA
atgcaaggtacacactggtacaca

SEQ ID NO: 128 - protein, 8 aa, synthetic construct
  REGION 1..8: note = synthetic
MQGTHWYT

SEQ ID NO: 129 - DNA, 357 bp, synthetic construct
  misc_feature 1..357: note = synthetic
  source 1..357: mol_type = other DNA
caggtgcagctgcagcagtggggcgcaggactattgaagccttcggagaccctgtccctcacctgcgctgtctatggtgggtctttcagtggttattactggagctggatccgccagcccccagggaagggtctggaatggattggggaaatcaatcatagaggaaacaccaactacaacccgtccctcaagagtcgagtcaccatatcactcgacacgtccaagaaacagttctccctgaacctgagttctgtgaccgccgcggacacggctatgtattactgtacgagagacgaagaacaggaactacgtttccttgactactggggccagggaaccctggtcaccgtctcctca

SEQ ID NO: 130 - protein, 119 aa, synthetic construct
  REGION 1..119: note = synthetic
QVQLQQWGAGLLKPSETLSLTCAVYGGSFSGYYWSWIRQPPGKGLEWIGEINHRGNTNYNPSLKSRVTISLDTSKKQFSLNLSSVTAADTAMYYCTRDEEQELRFLDYWGQGTLVTVSS

SEQ ID NO: 131 - DNA, 24 bp, synthetic construct
  misc_feature 1..24: note = synthetic
  source 1..24: mol_type = other DNA
ggtgggtctttcagtggttattac

SEQ ID NO: 132 - protein, 8 aa, synthetic construct
  REGION 1..8: note = synthetic
GGSFSGYY

SEQ ID NO: 133 - DNA, 21 bp, synthetic construct
  misc_feature 1..21: note = synthetic
  source 1..21: mol_type = other DNA
atcaatcatagaggaaacacc

SEQ ID NO: 134 - protein, 7 aa, synthetic construct
  REGION 1..7: note = synthetic
INHRGNT

SEQ ID NO: 135 - DNA, 39 bp, synthetic construct
  misc_feature 1..39: note = synthetic
  source 1..39: mol_type = other DNA
acgagagacgaagaacaggaactacgtttccttgactac

SEQ ID NO: 136 - protein, 13 aa, synthetic construct
  REGION 1..13: note = synthetic
TRDEEQELRFLDY

SEQ ID NO: 137 - DNA, 321 bp, synthetic construct
  misc_feature 1..321: note = synthetic
  source 1..321: mol_type = other DNA
gagattgtgttgacacagtctccagccaccctgtctttgtctccaggggaaagagccaccctctcctgcagggccagtcaggatattagcacctacttagcctggtaccaacagagagctggccaggctcccaggctcctcatctatggtgcttccaacagggccactggcatcccagccaggttcagtggcagtgggtctgggacagacttcactctcaccatcagcagcctagagcctgaagattttgcattttattactgtcaacagcgcagcaactggccgctcactttcggcggagggaccgaggtggagatcaaa

SEQ ID NO: 138 - protein, 107 aa, synthetic construct
  REGION 1..107: note = synthetic
EIVLTQSPATLSLSPGERATLSCRASQDISTYLAWYQQRAGQAPRLLIYGASNRATGIPARFSGSGSGTDFTLTISSLEPEDFAFYYCQQRSNWPLTFGGGTEVEIK

SEQ ID NO: 139 - DNA, 18 bp, synthetic construct
  misc_feature 1..18: note = synthetic
  source 1..18: mol_type = other DNA
caggatattagcacctac

SEQ ID NO: 140 - protein, 6 aa, synthetic construct
  REGION 1..6: note = synthetic
QDISTY

SEQ ID NO: 141-142 - skipped (2 entries)

SEQ ID NO: 143 - DNA, 27 bp, synthetic construct
  misc_feature 1..27: note = synthetic
  source 1..27: mol_type = other DNA
caacagcgcagcaactggccgctcact

SEQ ID NO: 144 - protein, 9 aa, synthetic construct
  REGION 1..9: note = synthetic
QQRSNWPLT

SEQ ID NO: 145 - DNA, 366 bp, synthetic construct
  misc_feature 1..366: note = synthetic
  source 1..366: mol_type = other DNA
caggtgcagctacagcagtggggcgcaggactgttgaagccttcggagaccctgtccctcacctgcgttgtccatggtgggtccttcagtggttactactggaactggatccgccagcccccagggaaggggctggagtggattggggaaatcaatcatagaggaaacaccaactacaacccgtccctcaagagtcgagtcaccgtatcagaagacacgtccaagaaccagttctccctgaagctgagctctttgaccgccgcggacacggctgtgtattactgtgtgagaggagaggattacgatttttggagtgattattataatgactactggggccagggaaccctggtcaccgtctcctca

SEQ ID NO: 146 - protein, 122 aa, synthetic construct
  REGION 1..122: note = synthetic
QVQLQQWGAGLLKPSETLSLTCVVHGGSFSGYYWNWIRQPPGKGLEWIGEINHRGNTNYNPSLKSRVTVSEDTSKNQFSLKLSSLTAADTAVYYCVRGEDYDFWSDYYNDYWGQGTLVTVSS

SEQ ID NO: 147 - DNA, 24 bp, synthetic construct
  misc_feature 1..24: note = synthetic
  source 1..24: mol_type = other DNA
ggtgggtccttcagtggttactac

SEQ ID NO: 148 - protein, 8 aa, synthetic construct
  REGION 1..8: note = synthetic
GGSFSGYY

SEQ ID NO: 149 - DNA, 21 bp, synthetic construct
  misc_feature 1..21: note = synthetic
  source 1..21: mol_type = other DNA
atcaatcatagaggaaacacc

SEQ ID NO: 150 - protein, 7 aa, synthetic construct
  REGION 1..7: note = synthetic
INHRGNT

SEQ ID NO: 151 - DNA, 48 bp, synthetic construct
  misc_feature 1..48: note = synthetic
  source 1..48: mol_type = other DNA
gtgagaggagaggattacgatttttggagtgattattataatgactac

SEQ ID NO: 152 - protein, 16 aa, synthetic construct
  REGION 1..16: note = synthetic
VRGEDYDFWSDYYNDY

SEQ ID NO: 153 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcagactattagcagctacttagcctggcaccaacagaaacctggccaggctcccaggctcctcatctatgatgcatccaaaagggccacgggcatcccagccaggttcagtggcagtgggtctgggacagacttcactctcaccatcaccagcctagagcctgaagattttgcagtttattactgtcagcagcgtagcaactggcctctcactttcggcggagggaccaaggtggagatcaaa

SEQ ID NO: 154 - protein, 107 aa, synthetic construct
  REGION 1..107: note = synthetic
EIVLTQSPATLSLSPGERATLSCRASQTISSYLAWHQQKPGQAPRLLIYDASKRATGIPARFSGSGSGTDFTLTITSLEPEDFAVYYCQQRSNWPLTFGGGTKVEIK

SEQ ID NO: 155 - DNA, 18 bp, synthetic construct
  misc_feature 1..18: note = synthetic
  source 1..18: mol_type = other DNA
cagactattagcagctac

SEQ ID NO: 156 - protein, 6 aa, synthetic construct
  REGION 1..6: note = synthetic
QTISSY

SEQ ID NO: 157-158 - skipped (2 entries)

SEQ ID NO: 159 - DNA, 27 bp, synthetic construct
  misc_feature 1..27: note = synthetic
  source 1..27: mol_type = other DNA
cagcagcgtagcaactggcctctcact

SEQ ID NO: 160 - protein, 9 aa, synthetic construct
  REGION 1..9: note = synthetic
QQRSNWPLT

SEQ ID NO: 161 - DNA, 372 bp, synthetic construct
  misc_feature 1..372: note = synthetic
  source 1..372: mol_type = other DNA
caggtgcagctacagcagtggggcgcaggactgttgccgccttcggagaccctgtccctcatctgcgctgtctatggtgggtccttcagtggttactactggagctggatccgccagcccccagggaaggggctggagtggattggggaaatcaatcatagaggaagcaccaactacaacccgtccctcaagagtcgagccaccatatcagttgacacgtccaagaaccagttctccctgaagctgagctctgtgaccgccgcggacacggctgtgtattactgttcgagaggcgaggattactatgatagtagtggttactcgtactactttgactactggggccagggaaccctggtcaccgtctcctca

SEQ ID NO: 162 - protein, 124 aa, synthetic construct
  REGION 1..124: note = synthetic
QVQLQQWGAGLLPPSETLSLICAVYGGSFSGYYWSWIRQPPGKGLEWIGEINHRGSTNYNPSLKSRATISVDTSKNQFSLKLSSVTAADTAVYYCSRGEDYYDSSGYSYYFDYWGQGTLVTVSS

SEQ ID NO: 163 - DNA, 24 bp, synthetic construct
  misc_feature 1..24: note = synthetic
  source 1..24: mol_type = other DNA
ggtgggtccttcagtggttactac

SEQ ID NO: 164 - protein, 8 aa, synthetic construct
  REGION 1..8: note = synthetic
GGSFSGYY

SEQ ID NO: 165 - DNA, 21 bp, synthetic construct
  misc_feature 1..21: note = synthetic
  source 1..21: mol_type = other DNA
atcaatcatagaggaagcacc

SEQ ID NO: 166 - protein, 7 aa, synthetic construct
  REGION 1..7: note = synthetic
INHRGST

SEQ ID NO: 167 - DNA, 54 bp, synthetic construct
  misc_feature 1..54: note = synthetic
  source 1..54: mol_type = other DNA
tcgagaggcgaggattactatgatagtagtggttactcgtactactttgactac

SEQ ID NO: 168 - protein, 18 aa, synthetic construct
  REGION 1..18: note = synthetic
SRGEDYYDSSGYSYYFDY

SEQ ID NO: 169 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgctcactttcggcggagggaccaaggtggagatcaaa

SEQ ID NO: 170 - protein, 107 aa, synthetic construct
  REGION 1..107: note = synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 171 - DNA, 18 bp, synthetic construct
  misc_feature 1..18: note = synthetic
  source 1..18: mol_type = other DNA
cagagtgttagcagctac

SEQ ID NO: 172 - protein, 6 aa, synthetic construct
  REGION 1..6: note = synthetic
QSVSSY

SEQ ID NO: 173-174 - skipped (2 entries)

SEQ ID NO: 175 - DNA, 27 bp, synthetic construct
  misc_feature 1..27: note = synthetic
  source 1..27: mol_type = other DNA
cagcagcgtagcaactggccgctcact

SEQ ID NO: 176 - protein, 9 aa, synthetic construct
  REGION 1..9: note = synthetic
QQRSNWPLT

SEQ ID NO: 177 - DNA, 366 bp, synthetic construct
  misc_feature 1..366: note = synthetic
  source 1..366: mol_type = other DNA
caggtgcagctacagcagtggggcgcaggactgttgaggccttcggagaccctgtccctcacctgcgctgtctatggtgggtccttcagtggttactactggaattggatccgccagtccccagggacggggctggagtggattggggaaatcaatcatagagggaacatcaacttcaacccgtccctcaagagtcgagtcaccatatcagaggacacgtccaaaaaccaattctccctgaggctgaactctgtgaccgccgcggacacggctgtgtattactgtgcgagaggagaggattacgatatttggagtggttattatagggagtactggggccagggaaccctggtcaccgtctcctca

SEQ ID NO: 178 - protein, 122 aa, synthetic construct
  REGION 1..122: note = synthetic
QVQLQQWGAGLLRPSETLSLTCAVYGGSFSGYYWNWIRQSPGTGLEWIGEINHRGNINFNPSLKSRVTISEDTSKNQFSLRLNSVTAADTAVYYCARGEDYDIWSGYYREYWGQGTLVTVSS

SEQ ID NO: 179 - DNA, 24 bp, synthetic construct
  misc_feature 1..24: note = synthetic
  source 1..24: mol_type = other DNA
ggtgggtccttcagtggttactac

SEQ ID NO: 180 - protein, 8 aa, synthetic construct
  REGION 1..8: note = synthetic
GGSFSGYY

SEQ ID NO: 181 - DNA, 21 bp, synthetic construct
  misc_feature 1..21: note = synthetic
  source 1..21: mol_type = other DNA
atcaatcatagagggaacatc

SEQ ID NO: 182 - protein, 7 aa, synthetic construct
  REGION 1..7: note = synthetic
INHRGNI

SEQ ID NO: 183 - DNA, 48 bp, synthetic construct
  misc_feature 1..48: note = synthetic
  source 1..48: mol_type = other DNA
gcgagaggagaggattacgatatttggagtggttattatagggagtac

SEQ ID NO: 184 - protein, 16 aa, synthetic construct
  REGION 1..16: note = synthetic
ARGEDYDIWSGYYREY

SEQ ID NO: 185 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccactctctcctgcagggccagtcagagtgttagcagctacttagcctggtaccagcagaaacctggccaggctcccaggctcctcatctatgatgcatccaagagggccactggcatcccagccaggttcagtggcagtgggtctgggacagacttcactctcaccatcagcagcctagagcctgaagattttgctgtttattactgtcagcagcgtagcaactggcctctcgctttcggcggagggaccaaggtggagatcaaa

SEQ ID NO: 186 - protein, 107 aa, synthetic construct
  REGION 1..107: note = synthetic
EIVLTQSPATLSLSPGERATLSCRASQSVSSYLAWYQQKPGQAPRLLIYDASKRATGIPARFSGSGSGTDFTLTISSLEPEDFAVYYCQQRSNWPLAFGGGTKVEIK

SEQ ID NO: 187 - DNA, 18 bp, synthetic construct
  misc_feature 1..18: note = synthetic
  source 1..18: mol_type = other DNA
cagagtgttagcagctac

SEQ ID NO: 188 - protein, 6 aa, synthetic construct
  REGION 1..6: note = synthetic
QSVSSY

SEQ ID NO: 189-190 - skipped (2 entries)

SEQ ID NO: 191 - DNA, 27 bp, synthetic construct
  misc_feature 1..27: note = synthetic
  source 1..27: mol_type = other DNA
cagcagcgtagcaactggcctctcgct

SEQ ID NO: 192 - protein, 9 aa, synthetic construct
  REGION 1..9: note = synthetic
QQRSNWPLA

SEQ ID NO: 193 - DNA, 357 bp, synthetic construct
  misc_feature 1..357: note = synthetic
  source 1..357: mol_type = other DNA
caggtgcagctacagcagtggggcgcaggactgttgaagccttcggagaccctgtccctcacctgcgctgtctatggtgggtccttcagtgagttctactggaactggatccgccagcccccagagaagggcctggagtggattggggaaatcaatcatcgtggaaacaccaactacaacccgtccctcaagagtcgagtcaccatatcagtagacatgtccaagaaccagttctccctgcagctgaactctgtgaccgtcgcggacacggctctgtattactgtgcgtttggctacgattttcggagttcttatgaggacgtctggggccaagggaccacggtcaccgtctcctca

SEQ ID NO: 194 - protein, 119 aa, synthetic construct
  REGION 1..119: note = synthetic
QVQLQQWGAGLLKPSETLSLTCAVYGGSFSEFYWNWIRQPPEKGLEWIGEINHRGNTNYNPSLKSRVTISVDMSKNQFSLQLNSVTVADTALYYCAFGYDFRSSYEDVWGQGTTVTVSS

SEQ ID NO: 195 - DNA, 24 bp, synthetic construct
  misc_feature 1..24: note = synthetic
  source 1..24: mol_type = other DNA
ggtgggtccttcagtgagttctac

SEQ ID NO: 196 - protein, 8 aa, synthetic construct
  REGION 1..8: note = synthetic
GGSFSEFY

SEQ ID NO: 197 - DNA, 21 bp, synthetic construct
  misc_feature 1..21: note = synthetic
  source 1..21: mol_type = other DNA
atcaatcatcgtggaaacacc

SEQ ID NO: 198 - protein, 7 aa, synthetic construct
  REGION 1..7: note = synthetic
INHRGNT

SEQ ID NO: 199 - DNA, 39 bp, synthetic construct
  misc_feature 1..39: note = synthetic
  source 1..39: mol_type = other DNA
gcgtttggctacgattttcggagttcttatgaggacgtc

SEQ ID NO: 200 - protein, 13 aa, synthetic construct
  REGION 1..13: note = synthetic
AFGYDFRSSYEDV

SEQ ID NO: 201 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcaggatattagcacctacttagcctggcaccaacagaaacctggccagcctcccaggctcctcatctatggttcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaa

SEQ ID NO: 202 - protein, 107 aa, synthetic construct
  REGION 1..107: note = synthetic
EIVLTQSPATLSLSPGERATLSCRASQDISTYLAWHQQKPGQPPRLLIYGSSNRATGIPARFSGSGSGTDFTLTISSLEPEDFAVYYCQQRSNWPLTFGGGTKVEIK

SEQ ID NO: 203 - DNA, 18 bp, synthetic construct
  misc_feature 1..18: note = synthetic
  source 1..18: mol_type = other DNA
caggatattagcacctac

SEQ ID NO: 204 - protein, 6 aa, synthetic construct
  REGION 1..6: note = synthetic
QDISTY

SEQ ID NO: 205-206 - skipped (2 entries)

SEQ ID NO: 207 - DNA, 27 bp, synthetic construct
  misc_feature 1..27: note = synthetic
  source 1..27: mol_type = other DNA
cagcagcgtagcaactggcctctcact

SEQ ID NO: 208 - protein, 9 aa, synthetic construct
  REGION 1..9: note = synthetic
QQRSNWPLT

SEQ ID NO: 209 - DNA, 372 bp, synthetic construct
  misc_feature 1..372: note = synthetic
  source 1..372: mol_type = other DNA
gaggtgcagctgttggagtctgggggaggcttggtacagccgggggggtccctgagactctcctgtgcagcctctggattcaccttcagaagctatgccatgagttgggtccgccaggctccagggaaggggctggagtgggtctcagttattagtggtggtggtggtaggacatactacacagactccgtgaagggccggttcaccatctccagagacaattccaagagcatgctgtatctgcaaatgaacagcctgagagccgaggacacggccatttattactgtgcgaaagagagggtaactggaatagaccactactactacggtgtggacgtctggggccaagggaccacggtcaccgtctcctca

SEQ ID NO: 210 - protein, 124 aa, synthetic construct
  REGION 1..124: note = synthetic
EVQLLESGGGLVQPGGSLRLSCAASGFTFRSYAMSWVRQAPGKGLEWVSVISGGGGRTYYTDSVKGRFTISRDNSKSMLYLQMNSLRAEDTAIYYCAKERVTGIDHYYYGVDVWGQGTTVTVSS

SEQ ID NO: 211 - DNA, 24 bp, synthetic construct
  misc_feature 1..24: note = synthetic
  source 1..24: mol_type = other DNA
ggattcaccttcagaagctatgcc

SEQ ID NO: 212 - protein, 8 aa, synthetic construct
  REGION 1..8: note = synthetic
GFTFRSYA

SEQ ID NO: 213 - DNA, 24 bp, synthetic construct
  misc_feature 1..24: note = synthetic
  source 1..24: mol_type = other DNA
attagtggtggtggtggtaggaca

SEQ ID NO: 214 - protein, 8 aa, synthetic construct
  REGION 1..8: note = synthetic
ISGGGGRT

SEQ ID NO: 215 - DNA, 51 bp, synthetic construct
  misc_feature 1..51: note = synthetic
  source 1..51: mol_type = other DNA
gcgaaagagagggtaactggaatagaccactactactacggtgtggacgtc

SEQ ID NO: 216 - protein, 17 aa, synthetic construct
  REGION 1..17: note = synthetic
AKERVTGIDHYYYGVDV

SEQ ID NO: 217 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagagcattagtagctatttaaattggtatcagcagaaaccagggaaagcccctaagctcctgatctatgctacatccagtttgcaaagtggggtcccatcacggttcagtggcagtgcatctggaacagatttcactctcgccatcagcagtctgcaacctgaagattttgcaacttactactgtcaacagagttacactacccccctcactttcggcggagggaccaaggtggagatcaaa

SEQ ID NO: 218 - protein, 107 aa, synthetic construct
  REGION 1..107: note = synthetic
DIQMTQSPSSLSASVGDRVTITCRASQSISSYLNWYQQKPGKAPKLLIYATSSLQSGVPSRFSGSASGTDFTLAISSLQPEDFATYYCQQSYTTPLTFGGGTKVEIK

SEQ ID NO: 219 - DNA, 18 bp, synthetic construct
  misc_feature 1..18: note = synthetic
  source 1..18: mol_type = other DNA
cagagcattagtagctat

SEQ ID NO: 220 - protein, 6 aa, synthetic construct
  REGION 1..6: note = synthetic
QSISSY

SEQ ID NO: 221-222 - skipped (2 entries)

SEQ ID NO: 223 - DNA, 27 bp, synthetic construct
  misc_feature 1..27: note = synthetic
  source 1..27: mol_type = other DNA
caacagagttacactacccccctcact

SEQ ID NO: 224 - protein, 9 aa, synthetic construct
  REGION 1..9: note = synthetic
QQSYTTPLT

SEQ ID NO: 225 - DNA, 390 bp, synthetic construct
  misc_feature 1..390: note = synthetic
  source 1..390: mol_type = other DNA
gaggtgcagctggtggagtctgggggaggcttggtacaacctggagggtccctgagactttcctgtgcagcctctggatttacattcagcagttatgaaatgaactgggtccgccaggctccagggaaggggctggagtgggtttcatatatcagtagtagtggtaataccaaagactacgcaggctctgtgaagggccgagtcaccatctccagagacaacgccaagaacttactgtatctgcaaatgaacagcctgagagccgaggacacggctgtttatcactgtgcgagagatggagggcattacgatattttgactggttccatgtcctactactactacgctttggacgtctggggccaagggaccacggtcaccgtctcctca

SEQ ID NO: 226 - protein, 130 aa, synthetic construct
  REGION 1..130: note = synthetic
EVQLVESGGGLVQPGGSLRLSCAASGFTFSSYEMNWVRQAPGKGLEWVSYISSSGNTKDYAGSVKGRVTISRDNAKNLLYLQMNSLRAEDTAVYHCARDGGHYDILTGSMSYYYYALDVWGQGTTVTVSS

SEQ ID NO: 227 - DNA, 24 bp, synthetic construct
  misc_feature 1..24: note = synthetic
  source 1..24: mol_type = other DNA
ggatttacattcagcagttatgaa

SEQ ID NO: 228 - protein, 8 aa, synthetic construct
  REGION 1..8: note = synthetic
GFTFSSYE

SEQ ID NO: 229 - DNA, 24 bp, synthetic construct
  misc_feature 1..24: note = synthetic
  source 1..24: mol_type = other DNA
atcagtagtagtggtaataccaaa

SEQ ID NO: 230 - protein, 8 aa, synthetic construct
  REGION 1..8: note = synthetic
ISSSGNTK

SEQ ID NO: 231 - DNA, 69 bp, synthetic construct
  misc_feature 1..69: note = synthetic
  source 1..69: mol_type = other DNA
gcgagagatggagggcattacgatattttgactggttccatgtcctactactactacgctttggacgtc

SEQ ID NO: 232 - protein, 23 aa, synthetic construct
  REGION 1..23: note = synthetic
ARDGGHYDILTGSMSYYYYALDV

SEQ ID NO: 233 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 234 - protein, 108 aa, synthetic construct; identical to SEQ ID NO: 26
  REGION 1..108: note = synthetic

SEQ ID NO: 235 - DNA, 18 bp, synthetic construct
  misc_feature 1..18: note = synthetic
  source 1..18: mol_type = other DNA
cagagcattagcagctat

SEQ ID NO: 236 - protein, 6 aa, synthetic construct
  REGION 1..6: note = synthetic
QSISSY

SEQ ID NO: 237-238 - skipped (2 entries)

SEQ ID NO: 239 - DNA, 30 bp, synthetic construct; identical to SEQ ID NO: 31
  misc_feature 1..30: note = synthetic
  source 1..30: mol_type = other DNA

SEQ ID NO: 240 - protein, 10 aa, synthetic construct
  REGION 1..10: note = synthetic
QQSYSTPPIT

SEQ ID NO: 241 - DNA, 363 bp, synthetic construct
  misc_feature 1..363: note = synthetic
  source 1..363: mol_type = other DNA
gaggtgcagctggtggagtctgggggaggcttggtacagcctggggggtccctgagactctcctgtgcagcctctggattcacctttaaaacctatgccatgagctgggtccgccaggctccagggagggggctggagtgggtctcaggtattagtggtagtggtagtacctcatactacgcagactccgtgaagggccggttcaccatctccagagacaattacaagaagacgctgtctctgcaaatgaacagtctgagagccgaggacacggccgtttattactgtgcgctggatataatggcaacggtaggaggtctctttaacaactggggccagggaaccctggtcaccgtctcctca

SEQ ID NO: 242 - protein, 121 aa, synthetic construct
  REGION 1..121: note = synthetic
EVQLVESGGGLVQPGGSLRLSCAASGFTFKTYAMSWVRQAPGRGLEWVSGISGSGSTSYYADSVKGRFTISRDNYKKTLSLQMNSLRAEDTAVYYCALDIMATVGGLFNNWGQGTLVTVSS

SEQ ID NO: 243 - DNA, 24 bp, synthetic construct
  misc_feature 1..24: note = synthetic
  source 1..24: mol_type = other DNA
ggattcacctttaaaacctatgcc

SEQ ID NO: 244 - protein, 8 aa, synthetic construct
  REGION 1..8: note = synthetic
GFTFKTYA

SEQ ID NO: 245 - DNA, 24 bp, synthetic construct
  misc_feature 1..24: note = synthetic
  source 1..24: mol_type = other DNA
attagtggtagtggtagtacctca

SEQ ID NO: 246 - protein, 8 aa, synthetic construct
  REGION 1..8: note = synthetic
ISGSGSTS

SEQ ID NO: 247 - DNA, 42 bp, synthetic construct
  misc_feature 1..42: note = synthetic
  source 1..42: mol_type = other DNA
gcgctggatataatggcaacggtaggaggtctctttaacaac

SEQ ID NO: 248 - protein, 14 aa, synthetic construct
  REGION 1..14: note = synthetic
ALDIMATVGGLFNN

SEQ ID NO: 249 - DNA, 324 bp, synthetic construct
  misc_feature 1..324: note = synthetic
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 250 - protein, 108 aa, synthetic construct
  REGION 1..108: note = synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 251 - DNA, 21 bp, synthetic construct
  misc_feature 1..21: note = synthetic
  source 1..21: mol_type = other DNA
cagagtgttagcagcagctac

SEQ ID NO: 252 - protein, 7 aa, synthetic construct
  REGION 1..7: note = synthetic
QSVSSSY

SEQ ID NO: 253-254 - skipped (2 entries)

SEQ ID NO: 255 - DNA, 27 bp, synthetic construct
  misc_feature 1..27: note = synthetic
  source 1..27: mol_type = other DNA
cagcagtatggtagctcaccttggacg

SEQ ID NO: 256 - protein, 9 aa, synthetic construct
  REGION 1..9: note = synthetic
QQYGSSPWT

SEQ ID NO: 257 - DNA, 360 bp, synthetic construct
  misc_feature 1..360: note = synthetic
  source 1..360: mol_type = other DNA
caggtgcagctggtgcagtctggggctgaggtgaagaagcctgggtcctcggtgaaggtctcctgcaaggcttctggaggcaccttcagcagacatactatcagctgggtgcgacaggcccctggacaagggcttgagtggatgggagggatcatccctatctttggtacagcaaactacgcacacaagttccagggcagagtcacgattaccacggacgaatccacgagcacagcctacatggagctgagcagcctgagatctgaggacacggccgtatattattgtgcgagagccccttatacccgacaggggtacttcgatctctggggccgtggcaccctggtcaccgtctcctca

SEQ ID NO: 258 - protein, 120 aa, synthetic construct
  REGION 1..120: note = synthetic
QVQLVQSGAEVKKPGSSVKVSCKASGGTFSRHTISWVRQAPGQGLEWMGGIIPIFGTANYAHKFQGRVTITTDESTSTAYMELSSLRSEDTAVYYCARAPYTRQGYFDLWGRGTLVTVSS

SEQ ID NO: 259 - DNA, 24 bp, synthetic construct
  misc_feature 1..24: note = synthetic
  source 1..24: mol_type = other DNA
ggaggcaccttcagcagacatact

SEQ ID NO: 260 - protein, 8 aa, synthetic construct
  REGION 1..8: note = synthetic
GGTFSRHT

SEQ ID NO: 261 - DNA, 24 bp, synthetic construct
  misc_feature 1..24: note = synthetic
  source 1..24: mol_type = other DNA
atcatccctatctttggtacagca

SEQ ID NO: 262 - protein, 8 aa, synthetic construct
  REGION 1..8: note = synthetic
IIPIFGTA

SEQ ID NO: 263 - DNA, 39 bp, synthetic construct
  misc_feature 1..39: note = synthetic
  source 1..39: mol_type = other DNA
gcgagagccccttatacccgacaggggtacttcgatctc

SEQ ID NO: 264 - protein, 13 aa, synthetic construct
  REGION 1..13: note = synthetic
ARAPYTRQGYFDL

SEQ ID NO: 265 - DNA, 339 bp, synthetic construct
  misc_feature 1..339: note = synthetic
  source 1..339: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctactcatttactgggcatctacccgggaatccggggtccctgaccgattcagtggcagcgggtctgggacagatttcactctcaccatcagcagcctgcaggctgaagatgtggcagtttattactgtcagcaagattatagtactccgtggacgttcggccaagggaccaaggtggaaatcaaa

SEQ ID NO: 266 - protein, 113 aa, synthetic construct
  REGION 1..113: note = synthetic
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQDYSTPWTFGQGTKVEIK

SEQ ID NO: 267 - DNA, 36 bp, synthetic construct
  misc_feature 1..36: note = synthetic
  source 1..36: mol_type = other DNA
cagagtgttttatacagctccaacaataagaactac

SEQ ID NO: 268 - protein, 12 aa, synthetic construct
  REGION 1..12: note = synthetic
QSVLYSSNNKNY

SEQ ID NO: 269-270 - skipped (2 entries)

SEQ ID NO: 271 - DNA, 27 bp, synthetic construct
  misc_feature 1..27: note = synthetic
  source 1..27: mol_type = other DNA
cagcaagattatagtactccgtggacg

SEQ ID NO: 272 - protein, 9 aa, synthetic construct
  REGION 1..9: note = synthetic
QQDYSTPWT

SEQ ID NO: 273 - DNA, 354 bp, synthetic construct
  misc_feature 1..354: note = synthetic
  source 1..354: mol_type = other DNA
caggtgcagctggtgcagtctggggctgaggtgaagaagcctggggcctcagtgaaggtttcctgcaaggcatctggatacaccttcaccaactactatatacactgggtgcgacaggcccctggacaagggcttgactggatgggaattatcaaccctggtggtggtaacacaaactacgcacagaagttcctgggcagagtcaccatgaccagggacacgtccacgaccacagtctacatggagctgagcagcctgagatctgaggacacggccatatattactgtgcgagagaaaactggaactcttactttgacaactggggccagggaaccctggtcaccgtctcctca

SEQ ID NO: 274 - protein, 118 aa, synthetic construct
  REGION 1..118: note = synthetic
QVQLVQSGAEVKKPGASVKVSCKASGYTFTNYYIHWVRQAPGQGLDWMGIINPGGGNTNYAQKFLGRVTMTRDTSTTTVYMELSSLRSEDTAIYYCARENWNSYFDNWGQGTLVTVSS

SEQ ID NO: 275 - DNA, 24 bp, synthetic construct
  misc_feature 1..24: note = synthetic
  source 1..24: mol_type = other DNA
ggatacaccttcaccaactactat

SEQ ID NO: 276 - protein, 8 aa, synthetic construct
  REGION 1..8: note = synthetic
GYTFTNYY

SEQ ID NO: 277 - DNA, 24 bp, synthetic construct
  misc_feature 1..24: note = synthetic
  source 1..24: mol_type = other DNA
atcaaccctggtggtggtaacaca

SEQ ID NO: 278 - protein, 8 aa, synthetic construct
  REGION 1..8: note = synthetic
INPGGGNT

SEQ ID NO: 279 - DNA, 33 bp, synthetic construct
  misc_feature 1..33: note = synthetic
  source 1..33: mol_type = other DNA
gcgagagaaaactggaactcttactttgacaac

SEQ ID NO: 280 - protein, 11 aa, synthetic construct
  REGION 1..11: note = synthetic
ARENWNSYFDN

SEQ ID NO: 281 - DNA, 339 bp, synthetic construct
  misc_feature 1..339: note = synthetic
  source 1..339: mol_type = other DNA
gacatcgtgatgacccagtctccagactccctggctgtgtctctgggcgagagggccaccatcaactgcaagtccagccagagtgttttatacagctccaacaataagaacttcttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcactttattactgtcagcaatattatggtgctccgtggacgttcggccaagggaccaaggtggaaatcaaa

SEQ ID NO: 282 - protein, 113 aa, synthetic construct
  REGION 1..113: note = synthetic
DIVMTQSPDSLAVSLGERATINCKSSQSVLYSSNNKNFLAWYQQKPGQPPKLLIYWASTRESGVPDRFSGSGSGTDFTLTISSLQAEDVALYYCQQYYGAPWTFGQGTKVEIK

SEQ ID NO: 283 - DNA, 36 bp, synthetic construct
  misc_feature 1..36: note = synthetic
  source 1..36: mol_type = other DNA
cagagtgttttatacagctccaacaataagaacttc

SEQ ID NO: 284 - protein, 12 aa, synthetic construct
  REGION 1..12: note = synthetic
QSVLYSSNNKNF

SEQ ID NO: 285-286 - skipped (2 entries)

SEQ ID NO: 287 - DNA, 27 bp, synthetic construct
  misc_feature 1..27: note = synthetic
  source 1..27: mol_type = other DNA
cagcaatattatggtgctccgtggacg

SEQ ID NO: 288 - protein, 9 aa, synthetic construct
  REGION 1..9: note = synthetic
QQYYGAPWT

SEQ ID NO: 289 - DNA, 357 bp, synthetic construct
  misc_feature 1..357: note = synthetic
  source 1..357: mol_type = other DNA
caggtccagctggtgcagtctggggctgaggtgaagaagcctgggtcctcggtgaaggtctcctgcaaggcttctggaggcaccttcagcagctatactatcaactgggtgcgacaggcccctggacaagggcttgagtggatgggagggatcatccctatctttggtatagcaaactacgcacagaagttccagggcagagtcacgattaccacggacgaatccacgaacacagcctacatggagctgagcagcctgagatctgaggacacggccatttattactgtgcgagagcgagatatggttcggggagttatgactactggggccagggaaccctggtcaccgtctcctca

SEQ ID NO: 290 - protein, 119 aa, synthetic construct
  REGION 1..119: note = synthetic
QVQLVQSGAEVKKPGSSVKVSCKASGGTFSSYTINWVRQAPGQGLEWMGGIIPIFGIANYAQKFQGRVTITTDESTNTAYMELSSLRSEDTAIYYCARARYGSGSYDYWGQGTLVTVSS

SEQ ID NO: 291 - DNA, 24 bp, synthetic construct
  misc_feature 1..24: note = synthetic
  source 1..24: mol_type = other DNA
ggaggcaccttcagcagctatact

SEQ ID NO: 292 - protein, 8 aa, synthetic construct
  REGION 1..8: note = synthetic
GGTFSSYT

SEQ ID NO: 293 - DNA, 24 bp, synthetic construct
  misc_feature 1..24: note = synthetic
  source 1..24: mol_type = other DNA
atcatccctatctttggtatagca

SEQ ID NO: 294 - protein, 8 aa, synthetic construct
  REGION 1..8: note = synthetic
IIPIFGIA

SEQ ID NO: 295 - DNA, 36 bp, synthetic construct
  misc_feature 1..36: note = synthetic
  source 1..36: mol_type = other DNA
gcgagagcgagatatggttcggggagttatgactac

SEQ ID NO: 296 - protein, 12 aa, synthetic construct
  REGION 1..12: note = synthetic
ARARYGSGSYDY

SEQ ID NO: 297 - DNA, 339 bp, synthetic construct
  misc_feature 1..339: note = synthetic
  source 1..339: mol_type = other DNA
gacatcgtgatgacccagtctccagactccctggctgtgtctctgggcgagagggccaccatcaactgcaagtccagccagagtgttttatacacctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattataatactccatggacgttcggccaagggaccaaggtggaaatcaaa

SEQ ID NO: 298 - protein, 113 aa, synthetic construct
  REGION 1..113: note = synthetic
DIVMTQSPDSLAVSLGERATINCKSSQSVLYTSNNKNYLAWYQQKPGQPPKLLIYWASTRESGVPDRFSGSGSGTDFTLTISSLQAEDVAVYYCQQYYNTPWTFGQGTKVEIK

SEQ ID NO: 299 - DNA, 36 bp, synthetic construct
  misc_feature 1..36: note = synthetic
  source 1..36: mol_type = other DNA
cagagtgttttatacacctccaacaataagaactac

SEQ ID NO: 300 - protein, 12 aa, synthetic construct
  REGION 1..12: note = synthetic
QSVLYTSNNKNY

SEQ ID NO: 301-302 - skipped (2 entries)

SEQ ID NO: 303 - DNA, 27 bp, synthetic construct
  misc_feature 1..27: note = synthetic
  source 1..27: mol_type = other DNA
cagcaatattataatactccatggacg

SEQ ID NO: 304 - protein, 9 aa, synthetic construct
  REGION 1..9: note = synthetic
QQYYNTPWT

SEQ ID NO: 305 - DNA, 360 bp, synthetic construct
  misc_feature 1..360: note = synthetic
  source 1..360: mol_type = other DNA
cagatcaccttgaaggagtctggtcctacgctggtgaaacccacacagaccctcacgctgacctgcaccttctctgggttctcactcagcactaatggagtgggtgtgggctggatccgtcagcccccaggaaaggccctggagtggcttggaatcatttattggaatgatgataagcgctacagcccatctctgaggagcagactcaccatcaccaaggacacctccaaaaaccaggtggtccttacaatgaccaacatggaccctgtggacacagccacatattactgtgcacacagaggcctcttcggaggttggttcgacccctggggccagggaaccctggtcaccgtctcctca

SEQ ID NO: 306 - protein, 120 aa, synthetic construct
  REGION 1..120: note = synthetic
QITLKESGPTLVKPTQTLTLTCTFSGFSLSTNGVGVGWIRQPPGKALEWLGIIYWNDDKRYSPSLRSRLTITKDTSKNQVVLTMTNMDPVDTATYYCAHRGLFGGWFDPWGQGTLVTVSS

SEQ ID NO: 307 - DNA, 30 bp, synthetic construct
  misc_feature 1..30: note = synthetic
  source 1..30: mol_type = other DNA
gggttctcactcagcactaatggagtgggt

SEQ ID NO: 308 - protein, 10 aa, synthetic construct
  REGION 1..10: note = synthetic
GFSLSTNGVG

SEQ ID NO: 309 - DNA, 21 bp, synthetic construct
  misc_feature 1..21: note = synthetic
  source 1..21: mol_type = other DNA
atttattggaatgatgataag

SEQ ID NO: 310 - protein, 7 aa, synthetic construct
  REGION 1..7: note = synthetic
IYWNDDK

SEQ ID NO: 311 - DNA, 36 bp, synthetic construct
  misc_feature 1..36: note = synthetic
  source 1..36: mol_type = other DNA
gcacacagaggcctcttcggaggttggttcgacccc

SEQ ID NO: 312 - protein, 12 aa, synthetic construct
  REGION 1..12: note = synthetic
AHRGLFGGWFDP

SEQ ID NO: 313 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagagcattagcaggtatttaaattggtatcagcagaaaccagggaaagcccctaacctcctgatctttgctgcatccagtttgcaaagtggggtcccatcaaggttcagtggcagtggatctgggacagatttcactctcaccatcagcagtctgcaacctgaagattttgcaacttacttctgtcaacagagttacaataccccgctcactttcggcggagggaccaaggtggagatcaaa

SEQ ID NO: 314 - protein, 107 aa, synthetic construct
  REGION 1..107: note = synthetic
DIQMTQSPSSLSASVGDRVTITCRASQSISRYLNWYQQKPGKAPNLLIFAASSLQSGVPSRFSGSGSGTDFTLTISSLQPEDFATYFCQQSYNTPLTFGGGTKVEIK

SEQ ID NO: 315 - DNA, 18 bp, synthetic construct
  misc_feature 1..18: note = synthetic
  source 1..18: mol_type = other DNA
cagagcattagcaggtat

SEQ ID NO: 316 - protein, 6 aa, synthetic construct
  REGION 1..6: note = synthetic
QSISRY

SEQ ID NO: 317-318 - skipped (2 entries)

SEQ ID NO: 319 - DNA, 27 bp, synthetic construct
  misc_feature 1..27: note = synthetic
  source 1..27: mol_type = other DNA
caacagagttacaataccccgctcact

SEQ ID NO: 320 - protein, 9 aa, synthetic construct
  REGION 1..9: note = synthetic
QQSYNTPLT

SEQ ID NO: 321 - DNA, 366 bp, synthetic construct
  misc_feature 1..366: note = synthetic
  source 1..366: mol_type = other DNA
gaggtgcagctggtggagtctgggggaggcttggtacagccgggggggtccctgagactctcctgtgcaatctctggattcacctttaggagttatgccatgacctgggtccgccaggctccagggaaggcgctggagtgggtctcagttattagtggtagcggtggtaacacatactacgcagactccgtgaagggccggttcaccgtctccagagacaattccaggaacacgctgtatctgcaaatgaacagcctgagagccgaggacacggccgtatatttctgttcgaaagttgcagcagctaataattactattacgctttggacgtctggggccaagggaccacggtcaccgtctcctca

SEQ ID NO: 322 - protein, 122 aa, synthetic construct
  REGION 1..122: note = synthetic
EVQLVESGGGLVQPGGSLRLSCAISGFTFRSYAMTWVRQAPGKALEWVSVISGSGGNTYYADSVKGRFTVSRDNSRNTLYLQMNSLRAEDTAVYFCSKVAAANNYYYALDVWGQGTTVTVSS

SEQ ID NO: 323 - DNA, 24 bp, synthetic construct
  misc_feature 1..24: note = synthetic
  source 1..24: mol_type = other DNA
ggattcacctttaggagttatgcc

SEQ ID NO: 324 - protein, 8 aa, synthetic construct
  REGION 1..8: note = synthetic
GFTFRSYA

SEQ ID NO: 325 - DNA, 24 bp, synthetic construct
  misc_feature 1..24: note = synthetic
  source 1..24: mol_type = other DNA
attagtggtagcggtggtaacaca

SEQ ID NO: 326 - protein, 8 aa, synthetic construct
  REGION 1..8: note = synthetic
ISGSGGNT

SEQ ID NO: 327 - DNA, 45 bp, synthetic construct
  misc_feature 1..45: note = synthetic
  source 1..45: mol_type = other DNA
tcgaaagttgcagcagctaataattactattacgctttggacgtc

SEQ ID NO: 328 - protein, 15 aa, synthetic construct
  REGION 1..15: note = synthetic
SKVAAANNYYYALDV

SEQ ID NO: 329 - DNA, 336 bp, synthetic construct
  misc_feature 1..336: note = synthetic
  source 1..336: mol_type = other DNA
gatattgtgatgactcagtctccactctccctgcccgtcacccctggagagccggcctccatctcctgcaggtctagtcagagcctcctgcatagtaatggatacaagtatttggattggtacctgcagaagccagggcagtctccacaactcctgatctatttggtttctaatcgggcctccggggtccctgacaggttcagtggcagtggatcaggcacagattttacactgaaaatcagcagagtggaggctgaggatgttggggtttattattgcatgcaagctctacaaactccgtacacttttggccaggggaccaagctggagatcaaa

SEQ ID NO: 330 - protein, 112 aa, synthetic construct
  REGION 1..112: note = synthetic
DIVMTQSPLSLPVTPGEPASISCRSSQSLLHSNGYKYLDWYLQKPGQSPQLLIYLVSNRASGVPDRFSGSGSGTDFTLKISRVEAEDVGVYYCMQALQTPYTFGQGTKLEIK

SEQ ID NO: 331 - DNA, 33 bp, synthetic construct
  misc_feature 1..33: note = synthetic
  source 1..33: mol_type = other DNA
cagagcctcctgcatagtaatggatacaagtat

SEQ ID NO: 332 - protein, 11 aa, synthetic construct
  REGION 1..11: note = synthetic
QSLLHSNGYKY

SEQ ID NO: 333-334 - skipped (2 entries)

SEQ ID NO: 335 - DNA, 27 bp, synthetic construct
  misc_feature 1..27: note = synthetic
  source 1..27: mol_type = other DNA
atgcaagctctacaaactccgtacact

SEQ ID NO: 336 - protein, 9 aa, synthetic construct
  REGION 1..9: note = synthetic
MQALQTPYT

SEQ ID NO: 337 - DNA, 381 bp, synthetic construct
  misc_feature 1..381: note = synthetic
  source 1..381: mol_type = other DNA
caggtgcagctggtggagtctgggggaggcgtggtccagcctgggaggtccctgagactctcctgtgtagcgtctggattcaccttcagtaactatggcatgcactgggtccgccaggctccaggcaaggggctggagtgggtggcagttatatggaatgatggaagtaataaatactatgcagactccgtgaagggccgattcaccatctccagagacaattccaagaacacgctgtatctccaagtgagcagcctgagagccgatgacacggctgtatattactgtgcgagggacggagaggtcgaatatagcagctcgaattacaactactacggtctggatgtctggggccaagggaccacggtcaccgtctcctca

SEQ ID NO: 338 - protein, 127 aa, synthetic construct
  REGION 1..127: note = synthetic
QVQLVESGGGVVQPGRSLRLSCVASGFTFSNYGMHWVRQAPGKGLEWVAVIWNDGSNKYYADSVKGRFTISRDNSKNTLYLQVSSLRADDTAVYYCARDGEVEYSSSNYNYYGLDVWGQGTTVTVSS

SEQ ID NO: 339 - DNA, 24 bp, synthetic construct
  misc_feature 1..24: note = synthetic
  source 1..24: mol_type = other DNA
ggattcaccttcagtaactatggc

SEQ ID NO: 340 - protein, 8 aa, synthetic construct
  REGION 1..8: note = synthetic
GFTFSNYG

SEQ ID NO: 341 - DNA, 24 bp, synthetic construct
  misc_feature 1..24: note = synthetic
  source 1..24: mol_type = other DNA
atatggaatgatggaagtaataaa

SEQ ID NO: 342 - protein, 8 aa, synthetic construct
  REGION 1..8: note = synthetic
IWNDGSNK

SEQ ID NO: 343 - DNA, 60 bp, synthetic construct
  misc_feature 1..60: note = synthetic
  source 1..60: mol_type = other DNA
gcgagggacggagaggtcgaatatagcagctcgaattacaactactacggtctggatgtc

SEQ ID NO: 344 - protein, 20 aa, synthetic construct
  REGION 1..20: note = synthetic
ARDGEVEYSSSNYNYYGLDV

SEQ ID NO: 345 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaaactcctgatctacgatgcatccaatttggaaacaggggtcccatcaaggttcagtggaagtggatctgggacagattttactttcaccatcagcagcctgcagcctgaagatattgtaacatattactgtcaacagtatgatgatctcccgatcaccttcggccaagggacacgactggagattaaa

SEQ ID NO: 346 - protein, 107 aa, synthetic construct
  REGION 1..107: note = synthetic
DIQMTQSPSSLSASVGDRVTITCQASQDISNYLNWYQQKPGKAPKLLIYDASNLETGVPSRFSGSGSGTDFTFTISSLQPEDIVTYYCQQYDDLPITFGQGTRLEIK

SEQ ID NO: 347 - DNA, 18 bp, synthetic construct
  misc_feature 1..18: note = synthetic
  source 1..18: mol_type = other DNA
caggacattagcaactat

SEQ ID NO: 348 - protein, 6 aa, synthetic construct
  REGION 1..6: note = synthetic
QDISNY

SEQ ID NO: 349-350 - skipped (2 entries)

SEQ ID NO: 351 - DNA, 27 bp, synthetic construct
  misc_feature 1..27: note = synthetic
  source 1..27: mol_type = other DNA
caacagtatgatgatctcccgatcacc

SEQ ID NO: 352 - protein, 9 aa, synthetic construct
  REGION 1..9: note = synthetic
QQYDDLPIT

SEQ ID NO: 353 - DNA, 375 bp, synthetic construct
  misc_feature 1..375: note = synthetic
  source 1..375: mol_type = other DNA
gaggtgcagctggtggagtctgggggaggcttggtacagcctggggggtccctgagactctcctgtgcagcctctggattctcctttcataattttgccatgaactgggtccgccaggctccagggaaggggctggagtgggtctcagttattactggtagtggtactagcacacactacgcagactccgtgaagggccggttcaccatctccagagacaattccaagaaaacgctatatctgcaaatgaatagcctgagagccgaggacacggccgtatattactgtgcgaaagatcggggctatgattatagtggttcttactacaactggttcgacccctggggccagggaaccctggtcaccgtctcctca

SEQ ID NO: 354 - protein, 125 aa, synthetic construct
  REGION 1..125: note = synthetic
EVQLVESGGGLVQPGGSLRLSCAASGFSFHNFAMNWVRQAPGKGLEWVSVITGSGTSTHYADSVKGRFTISRDNSKKTLYLQMNSLRAEDTAVYYCAKDRGYDYSGSYYNWFDPWGQGTLVTVSS

SEQ ID NO: 355 - DNA, 24 bp, synthetic construct
  misc_feature 1..24: note = synthetic
  source 1..24: mol_type = other DNA
ggattctcctttcataattttgcc

SEQ ID NO: 356 - protein, 8 aa, synthetic construct
  REGION 1..8: note = synthetic
GFSFHNFA

SEQ ID NO: 357 - DNA, 24 bp, synthetic construct
  misc_feature 1..24: note = synthetic
  source 1..24: mol_type = other DNA
attactggtagtggtactagcaca

SEQ ID NO: 358 - protein, 8 aa, synthetic construct
  REGION 1..8: note = synthetic
ITGSGTST

SEQ ID NO: 359 - DNA, 54 bp, synthetic construct
  misc_feature 1..54: note = synthetic
  source 1..54: mol_type = other DNA
gcgaaagatcggggctatgattatagtggttcttactacaactggttcgacccc

SEQ ID NO: 360 - protein, 18 aa, synthetic construct
  REGION 1..18: note = synthetic
AKDRGYDYSGSYYNWFDP

SEQ ID NO: 361 - DNA, 327 bp, synthetic construct
  misc_feature 1..327: note = synthetic
  source 1..327: mol_type = other DNA
gacatccagatgacccagtctccatcctccctgtctgcatctgtaggagacagaatcaccatcacttgccgggcaagtcagagtattagcagctatttaaattggtatcagcagaaaccagggaaagcccctaaactcctgatctttgctgcatcaaatttgcaaagtggggtcccatcaaggttcagtggcagtggatctgggacagatttcactctcaccatcagtagtctgcaacctgaagattttgcaacttactactgtcaacagagttacagtaccccatccttattcactttcggccctgggaccaaagtggatatcaaa

SEQ ID NO: 362 - protein, 109 aa, synthetic construct
  REGION 1..109: note = synthetic
DIQMTQSPSSLSASVGDRITITCRASQSISSYLNWYQQKPGKAPKLLIFAASNLQSGVPSRFSGSGSGTDFTLTISSLQPEDFATYYCQQSYSTPSLFTFGPGTKVDIK

SEQ ID NO: 363 - DNA, 18 bp, synthetic construct
  misc_feature 1..18: note = synthetic
  source 1..18: mol_type = other DNA
cagagtattagcagctat

SEQ ID NO: 364 - protein, 6 aa, synthetic construct
  REGION 1..6: note = synthetic
QSISSY

SEQ ID NO: 365-366 - skipped (2 entries)

SEQ ID NO: 367 - DNA, 33 bp, synthetic construct
  misc_feature 1..33: note = synthetic
  source 1..33: mol_type = other DNA
caacagagttacagtaccccatccttattcact

SEQ ID NO: 368 - protein, 11 aa, synthetic construct
  REGION 1..11: note = synthetic
QQSYSTPSLFT

SEQ ID NO: 369 - DNA, 384 bp, synthetic construct
  misc_feature 1..384: note = synthetic
  source 1..384: mol_type = other DNA
gaggtgcagctggtggagtctgggggaggcttggtacagcctggagggtccctgagactctcctgtgcagtctctggattcaccttcagtagttacgagatgaactgggtccgccaggctccagggaaggggctggaatgggtttcacacattagtagtagtggaagtaccatatactacgcagactctgtgaagggccgattcaccatgtccagagacaacgccaagaactcactgtatctgcaaatgaacagcctgagagccgaggacacggctgtttattactgtgcgagagatgggaatatctggagtggttattatgccgcctactacttctacggtatggacgtctggggccaagggaccacggtcaccgtctcctca

SEQ ID NO: 370 - protein, 128 aa, synthetic construct
  REGION 1..128: note = synthetic
EVQLVESGGGLVQPGGSLRLSCAVSGFTFSSYEMNWVRQAPGKGLEWVSHISSSGSTIYYADSVKGRFTMSRDNAKNSLYLQMNSLRAEDTAVYYCARDGNIWSGYYAAYYFYGMDVWGQGTTVTVSS

SEQ ID NO: 371 - DNA, 24 bp, synthetic construct
  misc_feature 1..24: note = synthetic
  source 1..24: mol_type = other DNA
ggattcaccttcagtagttacgag

SEQ ID NO: 372 - protein, 8 aa, synthetic construct
  REGION 1..8: note = synthetic
GFTFSSYE

SEQ ID NO: 373 - DNA, 24 bp, synthetic construct
  misc_feature 1..24: note = synthetic
  source 1..24: mol_type = other DNA
attagtagtagtggaagtaccata

SEQ ID NO: 374 - protein, 8 aa, synthetic construct
  REGION 1..8: note = synthetic
ISSSGSTI

SEQ ID NO: 375 - DNA, 63 bp, synthetic construct
  misc_feature 1..63: note = synthetic
  source 1..63: mol_type = other DNA
gcgagagatgggaatatctggagtggttattatgccgcctactacttctacggtatggacgtc

SEQ ID NO: 376 - protein, 21 aa, synthetic construct
  REGION 1..21: note = synthetic
ARDGNIWSGYYAAYYFYGMDV

SEQ ID NO: 377 - DNA, 336 bp, synthetic construct
  misc_feature 1..336: note = synthetic
  source 1..336: mol_type = other DNA
gatattgtgatgacccagactccactctcctcacctgtcacccttggacagccggcctccatctcctgcaggtctagtcaaagcctcgtacacagtgatggaaaaacctacttgagttggcttcagcagaggccaggccagcctccaagactcctaatttataagatttctaaccggttctctggggtcccagacagaatcagtggcagtggggcagggacagatttcacactgaaaatcagcagggtggaagctgaggatgtcggggtttattactgcatgcaagctgtacaatttcctcggacgttcggccaagggaccaaggtggaaatcaaa

SEQ ID NO: 378 - protein, 112 aa, synthetic construct
  REGION 1..112: note = synthetic
DIVMTQTPLSSPVTLGQPASISCRSSQSLVHSDGKTYLSWLQQRPGQPPRLLIYKISNRFSGVPDRISGSGAGTDFTLKISRVEAEDVGVYYCMQAVQFPRTFGQGTKVEIK

SEQ ID NO: 379 - DNA, 33 bp, synthetic construct
  misc_feature 1..33: note = synthetic
  source 1..33: mol_type = other DNA
caaagcctcgtacacagtgatggaaaaacctac

SEQ ID NO: 380 - protein, 11 aa, synthetic construct
  REGION 1..11: note = synthetic
QSLVHSDGKTY

SEQ ID NO: 381-382 - skipped (2 entries)

SEQ ID NO: 383 - DNA, 27 bp, synthetic construct
  misc_feature 1..27: note = synthetic
  source 1..27: mol_type = other DNA
atgcaagctgtacaatttcctcggacg

SEQ ID NO: 384 - protein, 9 aa, synthetic construct
  REGION 1..9: note = synthetic
MQAVQFPRT

SEQ ID NO: 385 - DNA, 366 bp, synthetic construct
  misc_feature 1..366: note = synthetic
  source 1..366: mol_type = other DNA
caggtgcagctacagcagtggggcgcaggactgttgaacccttcggagaccctgtccctcacctgcgctgtctatggtggggccttcagtgattactactggaattggatccgccagcccccagggaaggggctggagtggattggggaaatcaatcatcgcggaagcaccaactacaacccgtccctcaagagtcgtgtcaccatttcagttgacacgtccaagaaccagttctccctgaggatgagctctgtgaccgccgcggacgcggctgtgtattactgtgcgagaggagaggattacgatatttggaatggttattatcaggaaaaatggggccagggaaccctggtcaccgtctcctca

SEQ ID NO: 386 - protein, 122 aa, synthetic construct
  REGION 1..122: note = synthetic
QVQLQQWGAGLLNPSETLSLTCAVYGGAFSDYYWNWIRQPPGKGLEWIGEINHRGSTNYNPSLKSRVTISVDTSKNQFSLRMSSVTAADAAVYYCARGEDYDIWNGYYQEKWGQGTLVTVSS

SEQ ID NO: 387 - DNA, 24 bp, synthetic construct
  misc_feature 1..24: note = synthetic
  source 1..24: mol_type = other DNA
ggtggggccttcagtgattactac

SEQ ID NO: 388 - protein, 8 aa, synthetic construct
  REGION 1..8: note = synthetic
GGAFSDYY

SEQ ID NO: 389 - DNA, 21 bp, synthetic construct
  misc_feature 1..21: note = synthetic
  source 1..21: mol_type = other DNA
atcaatcatcgcggaagcacc

SEQ ID NO: 390 - protein, 7 aa, synthetic construct
  REGION 1..7: note = synthetic
INHRGST

SEQ ID NO: 391 - DNA, 48 bp, synthetic construct
  misc_feature 1..48: note = synthetic
  source 1..48: mol_type = other DNA
gcgagaggagaggattacgatatttggaatggttattatcaggaaaaa

SEQ ID NO: 392 - protein, 16 aa, synthetic construct
  REGION 1..16: note = synthetic
ARGEDYDIWNGYYQEK

SEQ ID NO: 393 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcagagtattagcacctacttagcctggtaccaacagaagcctggccaggctcccaggctcctcatctatgatgcatccaagagggccactggcatcccagccaggttcagtggcagtgggtctgggacagacttcactctcaccatcagcagcctagagcctgaagattttgtagtttattactgtcaccagcgtagcaactggcctctcactttcggcggagggaccaaggtggagatcaaa

SEQ ID NO: 394 - protein, 107 aa, synthetic construct
  REGION 1..107: note = synthetic
EIVLTQSPATLSLSPGERATLSCRASQSISTYLAWYQQKPGQAPRLLIYDASKRATGIPARFSGSGSGTDFTLTISSLEPEDFVVYYCHQRSNWPLTFGGGTKVEIK

SEQ ID NO: 395 - DNA, 18 bp, synthetic construct
  misc_feature 1..18: note = synthetic
  source 1..18: mol_type = other DNA
cagagtattagcacctac

SEQ ID NO: 396 - protein, 6 aa, synthetic construct
  REGION 1..6: note = synthetic
QSISTY

SEQ ID NO: 397-398 - skipped (2 entries)

SEQ ID NO: 399 - DNA, 27 bp, synthetic construct
  misc_feature 1..27: note = synthetic
  source 1..27: mol_type = other DNA
caccagcgtagcaactggcctctcact

SEQ ID NO: 400 - protein, 9 aa, synthetic construct
  REGION 1..9: note = synthetic
HQRSNWPLT

SEQ ID NO: 401 - DNA, 351 bp, synthetic construct
  misc_feature 1..351: note = synthetic
  source 1..351: mol_type = other DNA
caggtgcagctgcaggagtcggggccaggactggtgaagccttcggagaccctgtccctcacctgcactgtctctggtggttccttcagtagttactactggagttggctccggcagcccccaggaaaggggctggagtggattggatatatcttttacagtgggagtaccgactacaacccctccctcaagagtcgagtcaccatttcagtagacacgtccaagaagcagttctccctgaagctgacctctgtgaccgctgcggacacggccgtctattactgtgcgcgaacaataagtacgtggtggttcgccccctggggccagggaaccctggtcaccgtctcctca

SEQ ID NO: 402 - protein, 117 aa, synthetic construct
  REGION 1..117: note = synthetic
QVQLQESGPGLVKPSETLSLTCTVSGGSFSSYYWSWLRQPPGKGLEWIGYIFYSGSTDYNPSLKSRVTISVDTSKKQFSLKLTSVTAADTAVYYCARTISTWWFAPWGQGTLVTVSS

SEQ ID NO: 403 - DNA, 24 bp, synthetic construct
  misc_feature 1..24: note = synthetic
  source 1..24: mol_type = other DNA
ggtggttccttcagtagttactac

SEQ ID NO: 404 - protein, 8 aa, synthetic construct
  REGION 1..8: note = synthetic
GGSFSSYY

SEQ ID NO: 405 - DNA, 21 bp, synthetic construct
  misc_feature 1..21: note = synthetic
  source 1..21: mol_type = other DNA
atcttttacagtgggagtacc

SEQ ID NO: 406 - protein, 7 aa, synthetic construct
  REGION 1..7: note = synthetic
IFYSGST

SEQ ID NO: 407 - DNA, 33 bp, synthetic construct
  misc_feature 1..33: note = synthetic
  source 1..33: mol_type = other DNA
gcgcgaacaataagtacgtggtggttcgccccc

SEQ ID NO: 408 - protein, 11 aa, synthetic construct
  REGION 1..11: note = synthetic
ARTISTWWFAP

SEQ ID NO: 409 - DNA, 318 bp, synthetic construct
  misc_feature 1..318: note = synthetic
  source 1..318: mol_type = other DNA
gaaatagtgatgacacagtctccagccaccctgtctgtgtctccagggggaagagccaccctctcctgcagggccagtcagagtgttagcaacaacgtagcctggtaccagcagaaacctggccaggctcccaggctcctcatctatggtgcatccaccagggccactggtatcccaggcaggttcagtggcagtgggtctggaacagagttcactctcaccatcagcagcctgcagtctgaagattttgcagtttattcctgtcagcagtataataactggctcactttcggcggagggaccaaggtggagatcaaa

SEQ ID NO: 410 - protein, 106 aa, synthetic construct
  REGION 1..106: note = synthetic
EIVMTQSPATLSVSPGGRATLSCRASQSVSNNVAWYQQKPGQAPRLLIYGASTRATGIPGRFSGSGSGTEFTLTISSLQSEDFAVYSCQQYNNWLTFGGGTKVEIK

SEQ ID NO: 411 - DNA, 18 bp, synthetic construct
  misc_feature 1..18: note = synthetic
  source 1..18: mol_type = other DNA
cagagtgttagcaacaac

SEQ ID NO: 412 - protein, 6 aa, synthetic construct
  REGION 1..6: note = synthetic
QSVSNN

SEQ ID NO: 413-414 - skipped (2 entries)

SEQ ID NO: 415 - DNA, 24 bp, synthetic construct
  misc_feature 1..24: note = synthetic
  source 1..24: mol_type = other DNA
cagcagtataataactggctcact

SEQ ID NO: 416 - protein, 8 aa, synthetic construct
  REGION 1..8: note = synthetic
QQYNNWLT

SEQ ID NO: 417 - DNA, 369 bp, synthetic construct
  misc_feature 1..369: note = synthetic
  source 1..369: mol_type = other DNA
caggtgcagctggtggagtctgggggaggcgtggtccagcctgggaggtccctgagactctcctgtgtagcgtctggattcactttcagtagttatggcatgcactgggtccgccaggctccaggcaaggggctggagtgggtggcaattatatggtatgatggaagtaataaatactatgcagactccgtgaagggccgattcaccatatccagagacaattccaagaacacacagtatctgcaaatgaacagcctgagagccgaggacacggctgtgtattactgtgcgtcagtagctacgtctggggacttcgactactacggtatggacgtctggggccaagggaccacggtcaccgtctcctca

SEQ ID NO: 418 - protein, 123 aa, synthetic construct
  REGION 1..123: note = synthetic
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSS

SEQ ID NO: 419 - DNA, 24 bp, synthetic construct
  misc_feature 1..24: note = synthetic
  source 1..24: mol_type = other DNA
ggattcactttcagtagttatggc

SEQ ID NO: 420 - protein, 8 aa, synthetic construct
  REGION 1..8: note = synthetic
GFTFSSYG

SEQ ID NO: 421 - DNA, 24 bp, synthetic construct
  misc_feature 1..24: note = synthetic
  source 1..24: mol_type = other DNA
atatggtatgatggaagtaataaa

SEQ ID NO: 422 - protein, 8 aa, synthetic construct
  REGION 1..8: note = synthetic
IWYDGSNK

SEQ ID NO: 423 - DNA, 48 bp, synthetic construct
  misc_feature 1..48: note = synthetic
  source 1..48: mol_type = other DNA
gcgtcagtagctacgtctggggacttcgactactacggtatggacgtc

SEQ ID NO: 424 - protein, 16 aa, synthetic construct
  REGION 1..16: note = synthetic
ASVATSGDFDYYGMDV

SEQ ID NO: 425 - DNA, 321 bp, synthetic construct
  misc_feature 1..321: note = synthetic
  source 1..321: mol_type = other DNA
gaaattgtgttgacacagtctccagccaccctgtctttgtctccaggggaaagaaccaccctctcctgcagggccagtcagagaattagcacctacttagcctggtatcaacagaaacctggccaggctcccaggctcctcatctatgatgcatccaaaagggccactggcatcccagccaggttcagtggtagtgggtctgggacaggcttcactctcaccatcagcagcctagagcctgaagattttgcagtttattactgtcagcagcgtagtaactggcctctcactttcggcggagggaccaaggtggagatcaaa

SEQ ID NO: 426 - protein, 107 aa, synthetic construct
  REGION 1..107: note = synthetic
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIK

SEQ ID NO: 427 - DNA, 18 bp, synthetic construct
  misc_feature 1..18: note = synthetic
  source 1..18: mol_type = other DNA
cagagaattagcacctac

SEQ ID NO: 428 - protein, 6 aa, synthetic construct
  REGION 1..6: note = synthetic
QRISTY

SEQ ID NO: 429-430 - skipped (2 entries)

SEQ ID NO: 431 - DNA, 27 bp, synthetic construct
  misc_feature 1..27: note = synthetic
  source 1..27: mol_type = other DNA
cagcagcgtagtaactggcctctcact

SEQ ID NO: 432 - protein, 9 aa, synthetic construct
  REGION 1..9: note = synthetic
QQRSNWPLT

SEQ ID NO: 433 - DNA, 357 bp, synthetic construct
  misc_feature 1..357: note = synthetic
  source 1..357: mol_type = other DNA
gaggtgcagctggtgcagtctggagcagaggtgagaaagcccggggagtctctgaagatctcctgtaagggttctggatacagctttactaactactggatcgtctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacgggatacgattttcccttcctatcccctctggggccagggaaccctggtcaccgtctcctca

SEQ ID NO: 434 - protein, 119 aa, synthetic construct
  REGION 1..119: note = synthetic
EVQLVQSGAEVRKPGESLKISCKGSGYSFTNYWIVWVRQMPGKGLEWMGIIYPGDSDTRYSPSFQGQVTISADKSISTAYLQWSSLKASDTAMYYCARRDTIFPSYPLWGQGTLVTVSS

SEQ ID NO: 435 - DNA, 24 bp, synthetic construct
  misc_feature 1..24: note = synthetic
  source 1..24: mol_type = other DNA
ggatacagctttactaactactgg

SEQ ID NO: 436 - protein, 8 aa, synthetic construct
  REGION 1..8: note = synthetic
GYSFTNYW

SEQ ID NO: 437 - DNA, 24 bp, synthetic construct
  misc_feature 1..24: note = synthetic
  source 1..24: mol_type = other DNA
atctatcctggtgactctgatacc

SEQ ID NO: 438 - protein, 8 aa, synthetic construct
  REGION 1..8: note = synthetic
IYPGDSDT

SEQ ID NO: 439 - DNA, 36 bp, synthetic construct
  misc_feature 1..36: note = synthetic
  source 1..36: mol_type = other DNA
gcgagacgggatacgattttcccttcctatcccctc

SEQ ID NO: 440 - protein, 12 aa, synthetic construct
  REGION 1..12: note = synthetic
ARRDTIFPSYPL

SEQ ID NO: 441 - DNA, 336 bp, synthetic construct
  misc_feature 1..336: note = synthetic
  source 1..336: mol_type = other DNA
gatattgtgatgactcagtctcctctctccctgcccgtcacccctggagagccggcctccatctcctgcaggtctagtcagagcctcctgaatagtaatggatacaactttttggattggtacctgcagaagccagggcagtctccacaactcctgatctatttggtttctaatcgggcctccggggtccctgacaggttcagtggcagtggatcaggcacagattttacactgaaaatcagcagagtggaggctgaggatattggggtttattactgcatgcaagctctccaaactccgatcaccttcggccaagggacacgactggagattaaa

SEQ ID NO: 442 - protein, 112 aa, synthetic construct
  REGION 1..112: note = synthetic
DIVMTQSPLSLPVTPGEPASISCRSSQSLLNSNGYNFLDWYLQKPGQSPQLLIYLVSNRASGVPDRFSGSGSGTDFTLKISRVEAEDIGVYYCMQALQTPITFGQGTRLEIK

SEQ ID NO: 443 - DNA, 33 bp, synthetic construct
  misc_feature 1..33: note = synthetic
  source 1..33: mol_type = other DNA
cagagcctcctgaatagtaatggatacaacttt

SEQ ID NO: 444 - protein, 11 aa, synthetic construct
  REGION 1..11: note = synthetic
QSLLNSNGYNF

SEQ ID NO: 445-446 - skipped (2 entries)

SEQ ID NO: 447 - DNA, 27 bp, synthetic construct
  misc_feature 1..27: note = synthetic
  source 1..27: mol_type = other DNA
atgcaagctctccaaactccgatcacc

SEQ ID NO: 448 - protein, 9 aa, synthetic construct
  REGION 1..9: note = synthetic
MQALQTPIT

SEQ ID NO: 449 - DNA, 360 bp, synthetic construct
  misc_feature 1..360: note = synthetic
  source 1..360: mol_type = other DNA
cagatcaccttgaaggagtctggtcctacgctggtgaaacccacacagaccctcacgctgacctgcaccttctctgggttctcactcagcactaatggagtgggtgtgggctggatccgtcagcccccaggaaaggccctggagtggcttacactcatttattggaatgaaaataagcactacagcccatctctgaaaaacaggatcaccatcaccaaggacacctccaaaaaccaggtggtccttacaatgaccaacttggaccctgtggacacagccacttattactgtgtacacaggggatggttgggagcaatctttgcctactggggccagggaaccctggtcaccgtctcctca

SEQ ID NO: 450 - protein, 120 aa, synthetic construct
  REGION 1..120: note = synthetic
QITLKESGPTLVKPTQTLTLTCTFSGFSLSTNGVGVGWIRQPPGKALEWLTLIYWNENKHYSPSLKNRITITKDTSKNQVVLTMTNLDPVDTATYYCVHRGWLGAIFAYWGQGTLVTVSS

SEQ ID NO: 451 - DNA, 30 bp, synthetic construct; identical to SEQ ID NO: 307
  misc_feature 1..30: note = synthetic
  source 1..30: mol_type = other DNA

SEQ ID NO: 452 - protein, 10 aa, synthetic construct
  REGION 1..10: note = synthetic
GFSLSTNGVG

SEQ ID NO: 453 - DNA, 21 bp, synthetic construct
  misc_feature 1..21: note = synthetic
  source 1..21: mol_type = other DNA
atttattggaatgaaaataag

SEQ ID NO: 454 - protein, 7 aa, synthetic construct
  REGION 1..7: note = synthetic
IYWNENK

SEQ ID NO: 455 - DNA, 36 bp, synthetic construct
  misc_feature 1..36: note = synthetic
  source 1..36: mol_type = other DNA
gtacacaggggatggttgggagcaatctttgcctac

SEQ ID NO: 456 - protein, 12 aa, synthetic construct
  REGION 1..12: note = synthetic
VHRGWLGAIFAY

SEQ ID NO: 457 - DNA, 378 bp, synthetic construct
  misc_feature 1..378: note = synthetic
  source 1..378: mol_type = other DNA
gaggtgcagctggtggagtctgggggaggcttggtacagcctggggggtccctgagactctcctgtgcagcctctggattcacctttactagttatgccatgacctgggtccgccaggctccagggaaggggctggagtgggtctcagatattagtggtagtggtggtagaacatattacgcagactccgtgaagggccggttcaccatctccagagacaattccaagaatatgctgtatctgcaaatgaacatcctgagagccgaagacacggccgtatatcattgtgcgaagggaacaggccagcaggtggacctttacaactactactatgctttggacgtctggggccaagggaccacggtcaccgtctcctca

SEQ ID NO: 458 - protein, 126 aa, synthetic construct
  REGION 1..126: note = synthetic
EVQLVESGGGLVQPGGSLRLSCAASGFTFTSYAMTWVRQAPGKGLEWVSDISGSGGRTYYADSVKGRFTISRDNSKNMLYLQMNILRAEDTAVYHCAKGTGQQVDLYNYYYALDVWGQGTTVTVSS

SEQ ID NO: 459 - DNA, 24 bp, synthetic construct
  misc_feature 1..24: note = synthetic
  source 1..24: mol_type = other DNA
ggattcacctttactagttatgcc

SEQ ID NO: 460 - protein, 8 aa, synthetic construct
  REGION 1..8: note = synthetic
GFTFTSYA

SEQ ID NO: 461 - DNA, 24 bp, synthetic construct
  misc_feature 1..24: note = synthetic
  source 1..24: mol_type = other DNA
attagtggtagtggtggtagaaca

SEQ ID NO: 462 - protein, 8 aa, synthetic construct
  REGION 1..8: note = synthetic
ISGSGGRT

SEQ ID NO: 463 - DNA, 57 bp, synthetic construct
  misc_feature 1..57: note = synthetic
  source 1..57: mol_type = other DNA
gcgaagggaacaggccagcaggtggacctttacaactactactatgctttggacgtc

SEQ ID NO: 464 - protein, 19 aa, synthetic construct
  REGION 1..19: note = synthetic
AKGTGQQVDLYNYYYALDV

SEQ ID NO: 465 - DNA, 387 bp, synthetic construct
  misc_feature 1..387: note = synthetic
  source 1..387: mol_type = other DNA
caggtgcagctggtggagtctgggggaggcgtggtccagcctgggaggtccctgagactctcctgtgcagcgtctggattcaccttcagttactatggcatgcactgggtccgccaggctccaggcaaggggctggagtgggtggcagttatatggtatgatggaagtaataaacactatgcagactccgtgaagggccgattcaccatctccagagacaattccaagaacacgctgtatctgcaaatgaacagcctgagagccgacgacacggctgtctattactgtgcgagagataagggtataagtggaattaaggggggttcttactactactactatgccatggacgtctggggccaagggaccacggtcaccgtctcctca

SEQ ID NO: 466 - protein, 129 aa, synthetic construct
  REGION 1..129: note = synthetic
QVQLVESGGGVVQPGRSLRLSCAASGFTFSYYGMHWVRQAPGKGLEWVAVIWYDGSNKHYADSVKGRFTISRDNSKNTLYLQMNSLRADDTAVYYCARDKGISGIKGGSYYYYYAMDVWGQGTTVTVSS

SEQ ID NO: 467 - DNA, 24 bp, synthetic construct
  misc_feature 1..24: note = synthetic
  source 1..24: mol_type = other DNA
ggattcaccttcagttactatggc

SEQ ID NO: 468 - protein, 8 aa, synthetic construct
  REGION 1..8: note = synthetic
GFTFSYYG

SEQ ID NO: 469 - DNA, 24 bp, synthetic construct
  misc_feature 1..24: note = synthetic
  source 1..24: mol_type = other DNA
atatggtatgatggaagtaataaa

SEQ ID NO: 470 - protein, 8 aa, synthetic construct
  REGION 1..8: note = synthetic
IWYDGSNK

SEQ ID NO: 471 - DNA, 66 bp, synthetic construct
  misc_feature 1..66: note = synthetic
  source 1..66: mol_type = other DNA
gcgagagataagggtataagtggaattaaggggggttcttactactactactatgccatggacgtc

SEQ ID NO: 472 - protein, 22 aa, synthetic construct
  REGION 1..22: note = synthetic
ARDKGISGIKGGSYYYYYAMDV

SEQ ID NO: 473 - DNA, 360 bp, synthetic construct
  misc_feature 1..360: note = synthetic
  source 1..360: mol_type = other DNA
gaggtgcagctggtggagtctgggggaggcttggtaaagcctggggggtcccttagactctcctgtgcagcctctggattcactttcagtaacgcctggatgacctgggtccgccaggctccagggaaggggctggagtgggttggccgtattaaaaacaaaattgatggtgggacaacagactacgctgcacccgtgaaaggcagattcaccatctcaagagatgattcaaaaaacacggtttatctgcaaatgaacagcctgaaaaccgaggacacagccgtttattactgttccacggtggactacaattggtacttcgatttctggggccgtggcaccctggtcactgtctcctca

SEQ ID NO: 474 - protein, 120 aa, synthetic construct
  REGION 1..120: note = synthetic
EVQLVESGGGLVKPGGSLRLSCAASGFTFSNAWMTWVRQAPGKGLEWVGRIKNKIDGGTTDYAAPVKGRFTISRDDSKNTVYLQMNSLKTEDTAVYYCSTVDYNWYFDFWGRGTLVTVSS

SEQ ID NO: 475 - DNA, 24 bp, synthetic construct
  misc_feature 1..24: note = synthetic
  source 1..24: mol_type = other DNA
ggattcactttcagtaacgcctgg

SEQ ID NO: 476 - protein, 8 aa, synthetic construct
  REGION 1..8: note = synthetic
GFTFSNAW

SEQ ID NO: 477 - DNA, 30 bp, synthetic construct
  misc_feature 1..30: note = synthetic
  source 1..30: mol_type = other DNA
attaaaaacaaaattgatggtgggacaaca

SEQ ID NO: 478 - protein, 10 aa, synthetic construct
  REGION 1..10: note = synthetic
IKNKIDGGTT

SEQ ID NO: 479 - DNA, 33 bp, synthetic construct
  misc_feature 1..33: note = synthetic
  source 1..33: mol_type = other DNA
tccacggtggactacaattggtacttcgatttc

SEQ ID NO: 480 - protein, 11 aa, synthetic construct
  REGION 1..11: note = synthetic
STVDYNWYFDF

SEQ ID NO: 481 - DNA, 387 bp, synthetic construct
  misc_feature 1..387: note = synthetic
  source 1..387: mol_type = other DNA
caggtgcagctggtggagtctgggggaggcgtggtccagcctgggaggtccctgagactctcctgtgcagcgtctggattcaccttcagtttctttggcatgcactgggtccgccaggctccaggcaaggggctggagtgggtggcacttatatggtatgatggaactaatgaaaactatgcagactccgtgaagggccgattcaccatctccagagacaattccaagtccacgctgtatctgcaaatgaacagtctgagagccgaggacacggctgtttactactgtgcgagagataggggagtggcgacatttacgagggggaattactactacaactacggtatggacgtctggggccaagggaccacggtcaccgtctcctca

SEQ ID NO: 482 - protein, 129 aa, synthetic construct
  REGION 1..129: note = synthetic
QVQLVESGGGVVQPGRSLRLSCAASGFTFSFFGMHWVRQAPGKGLEWVALIWYDGTNENYADSVKGRFTISRDNSKSTLYLQMNSLRAEDTAVYYCARDRGVATFTRGNYYYNYGMDVWGQGTTVTVSS

SEQ ID NO: 483 - DNA, 24 bp, synthetic construct
  misc_feature 1..24: note = synthetic
  source 1..24: mol_type = other DNA
ggattcaccttcagtttctttggc

SEQ ID NO: 484 - protein, 8 aa, synthetic construct
  REGION 1..8: note = synthetic
GFTFSFFG

SEQ ID NO: 485 - DNA, 24 bp, synthetic construct
  misc_feature 1..24: note = synthetic
  source 1..24: mol_type = other DNA
atatggtatgatggaactaatgaa

SEQ ID NO: 486 - protein, 8 aa, synthetic construct
  REGION 1..8: note = synthetic
IWYDGTNE

SEQ ID NO: 487 - DNA, 66 bp, synthetic construct
  misc_feature 1..66: note = synthetic
  source 1..66: mol_type = other DNA
gcgagagataggggagtggcgacatttacgagggggaattactactacaactacggtatggacgtc

SEQ ID NO: 488 - protein, 22 aa, synthetic construct
  REGION 1..22: note = synthetic
ARDRGVATFTRGNYYYNYGMDV

SEQ ID NO: 489 - DNA, 387 bp, synthetic construct
  misc_feature 1..387: note = synthetic
  source 1..387: mol_type = other DNA
caggtgcagctggtggagtctgggggaggcgtggtccagcctgggaggtccctgagactctcctgtgcagcgtctggattcaccttcagtttctatggcatgcactgggtccgccaggctccaggcaaggggctggagggggtggcagttatatggtatgatggaagtaataaatactatgcagactccgtgaagggccgattcaccatatccagagacaattccaagaacatgctgtatctacaaatgaccagcctgagagccgaggacacggctgtgtattactgtgcgagagattcgggtaaaactggaactgggataactgggtactcctactactacggtatggacgtctggggccaagggaccacggtcaccgtctcctca

SEQ ID NO: 490 - protein, 129 aa, synthetic construct
  REGION 1..129: note = synthetic
QVQLVESGGGVVQPGRSLRLSCAASGFTFSFYGMHWVRQAPGKGLEGVAVIWYDGSNKYYADSVKGRFTISRDNSKNMLYLQMTSLRAEDTAVYYCARDSGKTGTGITGYSYYYGMDVWGQGTTVTVSS

SEQ ID NO: 491 - DNA, 24 bp, synthetic construct
  misc_feature 1..24: note = synthetic
  source 1..24: mol_type = other DNA
ggattcaccttcagtttctatggc

SEQ ID NO: 492 - protein, 8 aa, synthetic construct
  REGION 1..8: note = synthetic
GFTFSFYG

SEQ ID NO: 493 - DNA, 24 bp, synthetic construct
  misc_feature 1..24: note = synthetic
  source 1..24: mol_type = other DNA
atatggtatgatggaagtaataaa

SEQ ID NO: 494 - protein, 8 aa, synthetic construct
  REGION 1..8: note = synthetic
IWYDGSNK

SEQ ID NO: 495 - DNA, 66 bp, synthetic construct
  misc_feature 1..66: note = synthetic
  source 1..66: mol_type = other DNA
gcgagagattcgggtaaaactggaactgggataactgggtactcctactactacggtatggacgtc

SEQ ID NO: 496 - protein, 22 aa, synthetic construct
  REGION 1..22: note = synthetic
ARDSGKTGTGITGYSYYYGMDV

SEQ ID NO: 497 - DNA, 354 bp, synthetic construct
  misc_feature 1..354: note = synthetic
  source 1..354: mol_type = other DNA
cagctgcagctgcaggagtcgggcccaggactggtgaagccttcggagaccctgtccctcacctgcactgtctctggtggctccatcatcactaatagttattactggggctggatccgccagcccccagggaagggtctggagtggattggtagtatctattatagtgggaggacctactacaacccgtccctcgagagtcgagtcaccatatccgtggacacgtccaagaaccagttctccctgaagttgacctctgtgaccgccgcagacacggctatatattactgtgcgagggaaggggatccgtcgctcgacccctggggccagggaaccctggtcaccgtctcctca

SEQ ID NO: 498 - protein, 118 aa, synthetic construct
  REGION 1..118: note = synthetic
QLQLQESGPGLVKPSETLSLTCTVSGGSIITNSYYWGWIRQPPGKGLEWIGSIYYSGRTYYNPSLESRVTISVDTSKNQFSLKLTSVTAADTAIYYCAREGDPSLDPWGQGTLVTVSS

SEQ ID NO: 499 - DNA, 30 bp, synthetic construct
  misc_feature 1..30: note = synthetic
  source 1..30: mol_type = other DNA
ggtggctccatcatcactaatagttattac

SEQ ID NO: 500 - protein, 10 aa, synthetic construct
  REGION 1..10: note = synthetic
GGSIITNSYY

SEQ ID NO: 501 - DNA, 21 bp, synthetic construct
  misc_feature 1..21: note = synthetic
  source 1..21: mol_type = other DNA
atctattatagtgggaggacc

SEQ ID NO: 502 - protein, 7 aa, synthetic construct
  REGION 1..7: note = synthetic
IYYSGRT

SEQ ID NO: 503 - DNA, 30 bp, synthetic construct
  misc_feature 1..30: note = synthetic
  source 1..30: mol_type = other DNA
gcgagggaaggggatccgtcgctcgacccc

SEQ ID NO: 504 - protein, 10 aa, synthetic construct
  REGION 1..10: note = synthetic
AREGDPSLDP

SEQ ID NO: 505 - DNA, 363 bp, synthetic construct
  misc_feature 1..363: note = synthetic
  source 1..363: mol_type = other DNA
gaggtgcagctggtggagtctgggggagacttggtacagcctggggggtccctgagactctcctgtgcagcctctggattcacctttagcacctatgccatgaactgggtccgccaggctccagggaaggggctggagtgggtctcacatattagtggtagtggtggtaattcatactccgcagactccgtgaagggccggttcaccatctccagagacaattccaagaacacgctatatctgcaaatgaacagcctgcgagccgaggacacggccatatattactgttcgctggatataatggctacagtaggcggtctctttgcctactggggccagggaaccctggtcaccgtctcctca

SEQ ID NO: 506 - protein, 121 aa, synthetic construct
  REGION 1..121: note = synthetic
EVQLVESGGDLVQPGGSLRLSCAASGFTFSTYAMNWVRQAPGKGLEWVSHISGSGGNSYSADSVKGRFTISRDNSKNTLYLQMNSLRAEDTAIYYCSLDIMATVGGLFAYWGQGTLVTVSS

SEQ ID NO: 507 - DNA, 24 bp, synthetic construct
  misc_feature 1..24: note = synthetic
  source 1..24: mol_type = other DNA
ggattcacctttagcacctatgcc

SEQ ID NO: 508 - protein, 8 aa, synthetic construct
  REGION 1..8: note = synthetic
GFTFSTYA

SEQ ID NO: 509 - DNA, 24 bp, synthetic construct
  misc_feature 1..24: note = synthetic
  source 1..24: mol_type = other DNA
attagtggtagtggtggtaattca

SEQ ID NO: 510 - protein, 8 aa, synthetic construct
  REGION 1..8: note = synthetic
ISGSGGNS

SEQ ID NO: 511 - DNA, 42 bp, synthetic construct
  misc_feature 1..42: note = synthetic
  source 1..42: mol_type = other DNA
tcgctggatataatggctacagtaggcggtctctttgcctac

SEQ ID NO: 512 - protein, 14 aa, synthetic construct
  REGION 1..14: note = synthetic
SLDIMATVGGLFAY

SEQ ID NO: 513 - DNA, 393 bp, synthetic construct
  misc_feature 1..393: note = synthetic
  source 1..393: mol_type = other DNA
caggtgcagctggtggagtctgggggaggcgtggtccagcctgggaggtccctgagactctcctgtgtagcgtctggattcatcttcagtttctatggcatgcactgggtccgccaggctccagacaaggggctggagtgggtggcagttatatggtatgatggaagtaatgaatactatgcagactccgtgaagggccgattcaccatctccagagacaattccaagaacacgctgtatctgcaaatgaacagcctgagagccgaggacacggctgtgtattactgtgggagagatcaaggtatttcgtattacgatattttgactggtaattataactattactacggtgtggacgtctggggccaagggaccacggtcaccgtctcctca

SEQ ID NO: 514 - protein, 131 aa, synthetic construct
  REGION 1..131: note = synthetic
QVQLVESGGGVVQPGRSLRLSCVASGFIFSFYGMHWVRQAPDKGLEWVAVIWYDGSNEYYADSVKGRFTISRDNSKNTLYLQMNSLRAEDTAVYYCGRDQGISYYDILTGNYNYYYGVDVWGQGTTVTVSS

SEQ ID NO: 515 - DNA, 24 bp, synthetic construct
  misc_feature 1..24: note = synthetic
  source 1..24: mol_type = other DNA
ggattcatcttcagtttctatggc

SEQ ID NO: 516 - protein, 8 aa, synthetic construct
  REGION 1..8: note = synthetic
GFIFSFYG

SEQ ID NO: 517 - DNA, 24 bp, synthetic construct
  misc_feature 1..24: note = synthetic
  source 1..24: mol_type = other DNA
atatggtatgatggaagtaatgaa

SEQ ID NO: 518 - protein, 8 aa, synthetic construct
  REGION 1..8: note = synthetic
IWYDGSNE

SEQ ID NO: 519 - DNA, 72 bp, synthetic construct
  misc_feature 1..72: note = synthetic
  source 1..72: mol_type = other DNA
gggagagatcaaggtatttcgtattacgatattttgactggtaattataactattactacggtgtggacgtc

SEQ ID NO: 520 - protein, 24 aa, synthetic construct
  REGION 1..24: note = synthetic
GRDQGISYYDILTGNYNYYYGVDV

SEQ ID NO: 521 - DNA, 324 bp, synthetic construct; identical to SEQ ID NO: 233
  misc_feature 1..324: note = synthetic
  source 1..324: mol_type = other DNA

SEQ ID NO: 522 - protein, 108 aa, synthetic construct; identical to SEQ ID NO: 26
  REGION 1..108: note = synthetic

SEQ ID NO: 523 - DNA, 18 bp, synthetic construct
  misc_feature 1..18: note = synthetic
  source 1..18: mol_type = other DNA
cagagcattagcagctat

SEQ ID NO: 524 - protein, 6 aa, synthetic construct
  REGION 1..6: note = synthetic
QSISSY

SEQ ID NO: 525-526 - skipped (2 entries)

SEQ ID NO: 527 - DNA, 30 bp, synthetic construct; identical to SEQ ID NO: 31
  misc_feature 1..30: note = synthetic
  source 1..30: mol_type = other DNA

SEQ ID NO: 528 - protein, 10 aa, synthetic construct
  REGION 1..10: note = synthetic
QQSYSTPPIT

SEQ ID NO: 529 - DNA, 324 bp, synthetic construct; identical to SEQ ID NO: 249
  misc_feature 1..324: note = synthetic
  source 1..324: mol_type = other DNA

SEQ ID NO: 530 - protein, 108 aa, synthetic construct; identical to SEQ ID NO: 250
  REGION 1..108: note = synthetic

SEQ ID NO: 531 - DNA, 21 bp, synthetic construct
  misc_feature 1..21: note = synthetic
  source 1..21: mol_type = other DNA
cagagtgttagcagcagctac

SEQ ID NO: 532 - protein, 7 aa, synthetic construct
  REGION 1..7: note = synthetic
QSVSSSY

SEQ ID NO: 533-534 - skipped (2 entries)

SEQ ID NO: 535 - DNA, 27 bp, synthetic construct
  misc_feature 1..27: note = synthetic
  source 1..27: mol_type = other DNA
cagcagtatggtagctcaccttggacg

SEQ ID NO: 536 - protein, 9 aa, synthetic construct
  REGION 1..9: note = synthetic
QQYGSSPWT

SEQ ID NO: 537 - DNA, 357 bp, synthetic construct
  misc_feature 1..357: note = synthetic
  source 1..357: mol_type = other DNA
caggtgcagctacagcagtggggcgcaggactgttgaagccttcggagaccctgtccctcacctgcgctgtctatggtgggtccttcagtggttactactggaactggatccgccagcccccagggaaggggctggagtgggttggggaaatcagtcatagaggaagcaccaactacaacccgtccctcaagagtcgagtcaccatatcactggacacgtccaagaaccagttctccctgaagctgacctctgtgaccgccgcggacacggctgtgtattactgttcgagagacgaggaactggaattccgtttctttgactactggggccagggaaccctggtcaccgtctcctca

SEQ ID NO: 538 - protein, 119 aa, synthetic construct
  REGION 1..119: note = synthetic
QVQLQQWGAGLLKPSETLSLTCAVYGGSFSGYYWNWIRQPPGKGLEWVGEISHRGSTNYNPSLKSRVTISLDTSKNQFSLKLTSVTAADTAVYYCSRDEELEFRFFDYWGQGTLVTVSS

SEQ ID NO: 539 - DNA, 24 bp, synthetic construct
  misc_feature 1..24: note = synthetic
  source 1..24: mol_type = other DNA
ggtgggtccttcagtggttactac

SEQ ID NO: 540 - protein, 8 aa, synthetic construct
  REGION 1..8: note = synthetic
GGSFSGYY

SEQ ID NO: 541 - DNA, 21 bp, synthetic construct
  misc_feature 1..21: note = synthetic
  source 1..21: mol_type = other DNA
atcagtcatagaggaagcacc

SEQ ID NO: 542 - protein, 7 aa, synthetic construct
  REGION 1..7: note = synthetic
ISHRGST

SEQ ID NO: 543 - DNA, 39 bp, synthetic construct; identical to SEQ ID NO: 39
  misc_feature 1..39: note = synthetic
  source 1..39: mol_type = other DNA

SEQ ID NO: 544 - protein, 13 aa, synthetic construct
  REGION 1..13: note = synthetic
SRDEELEFRFFDY

SEQ ID NO: 545 - DNA, 321 bp, synthetic construct; identical to SEQ ID NO: 41
  misc_feature 1..321: note = synthetic
  source 1..321: mol_type = other DNA

SEQ ID NO: 546 - protein, 107 aa, synthetic construct; identical to SEQ ID NO: 42
  REGION 1..107: note = synthetic

SEQ ID NO: 547 - DNA, 18 bp, synthetic construct
  misc_feature 1..18: note = synthetic
  source 1..18: mol_type = other DNA
cagagtgttagcagctat

SEQ ID NO: 548 - protein, 6 aa, synthetic construct
  REGION 1..6: note = synthetic
QSVSSY

SEQ ID NO: 549-550 - skipped (2 entries)

SEQ ID NO: 551 - DNA, 27 bp, synthetic construct
  misc_feature 1..27: note = synthetic
  source 1..27: mol_type = other DNA
cagcagcgtagcaactggccgctcact

SEQ ID NO: 552 - protein, 9 aa, synthetic construct
  REGION 1..9: note = synthetic
QQRSNWPLT

SEQ ID NO: 553 - DNA, 372 bp, synthetic construct
  misc_feature 1..372: note = synthetic
  source 1..372: mol_type = other DNA
gaggtgcagctggtggagtctgggggaggcttggtacagcctggggggtccctgagactctcctgtgtagcctctggattcacctttagcctctatgccatgacctgggtccgccaggttccagggaaggggctggaatgggtctcaactattagtggtagtggtggtggcacatactacacagactccgttaagggccggttcaccatctccagagacaattccaagaacacactgtatctgcaaatgaacagcctgagagccgacgacacggccgttttttactgtacgaaagagagtacaactggaacttactcctacttctacggtatggacgtctggggccaagggaccacggtcaccgtctcctca

SEQ ID NO: 554 - protein, 124 aa, synthetic construct
  REGION 1..124: note = synthetic
EVQLVESGGGLVQPGGSLRLSCVASGFTFSLYAMTWVRQVPGKGLEWVSTISGSGGGTYYTDSVKGRFTISRDNSKNTLYLQMNSLRADDTAVFYCTKESTTGTYSYFYGMDVWGQGTTVTVSS

SEQ ID NO: 555 - DNA, 24 bp, synthetic construct
  misc_feature 1..24: note = synthetic
  source 1..24: mol_type = other DNA
ggattcacctttagcctctatgcc

SEQ ID NO: 556 - protein, 8 aa, synthetic construct
  REGION 1..8: note = synthetic
GFTFSLYA

SEQ ID NO: 557 - DNA, 24 bp, synthetic construct
  misc_feature 1..24: note = synthetic
  source 1..24: mol_type = other DNA
attagtggtagtggtggtggcaca

SEQ ID NO: 558 - protein, 8 aa, synthetic construct
  REGION 1..8: note = synthetic
ISGSGGGT

SEQ ID NO: 559 - DNA, 51 bp, synthetic construct
  misc_feature 1..51: note = synthetic
  source 1..51: mol_type = other DNA
acgaaagagagtacaactggaacttactcctacttctacggtatggacgtc

SEQ ID NO: 560 - protein, 17 aa, synthetic construct
  REGION 1..17: note = synthetic
TKESTTGTYSYFYGMDV

SEQ ID NO: 561 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagaccattagcagctatttaaattggtatcagcagaaaccagggaaagcccctaagctcctgatctatgctgcatccagtttgcaaagtggggtcccatcaaggttcagtggcagtggatctgggacagatttcactctcaccctcagcggtctccaacctgaagattttgcaacttactactgtcaacagagttacagtaccccgctcactttcggcggagggaccaaggtggagatcaaa

SEQ ID NO: 562 - protein, 107 aa, synthetic construct
  REGION 1..107: note = synthetic
DIQMTQSPSSLSASVGDRVTITCRASQTISSYLNWYQQKPGKAPKLLIYAASSLQSGVPSRFSGSGSGTDFTLTLSGLQPEDFATYYCQQSYSTPLTFGGGTKVEIK

SEQ ID NO: 563 - DNA, 18 bp, synthetic construct
  misc_feature 1..18: note = synthetic
  source 1..18: mol_type = other DNA
cagaccattagcagctat

SEQ ID NO: 564 - protein, 6 aa, synthetic construct
  REGION 1..6: note = synthetic
QTISSY

SEQ ID NO: 565-566 - skipped (2 entries)

SEQ ID NO: 567 - DNA, 27 bp, synthetic construct
  misc_feature 1..27: note = synthetic
  source 1..27: mol_type = other DNA
caacagagttacagtaccccgctcact

SEQ ID NO: 568 - protein, 9 aa, synthetic construct
  REGION 1..9: note = synthetic
QQSYSTPLT

SEQ ID NO: 569 - protein, 327 aa, synthetic construct
  REGION 1..327: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 570 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 571 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 572 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 573 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 574 - protein, 450 aa, synthetic construct
  REGION 1..450: note = aa 1-478: Human Lag3 (aa 29 through 450 of NP_002277.4) aa 451-478: myc-myc-hexahistidine tag
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLEQKLISEEDLGGEQKLISEEDLHHHHHH

SEQ ID NO: 575 - protein, 655 aa, synthetic construct
  REGION 1..655: note = aa 1-683: Human Lag3 (aa 29 through 450 of NP_002277.4) aa 451-683: mIgG2aFc
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 576 - protein, 516 aa, synthetic construct
  REGION 1..516: note = aa 1-533 (aa 18 through 533 of cynomolgus XP_005570011.1 modified to replace amino acid at position 74 with a P based on Rhesus macaque XP_001108923.1
APVKPPQPGAEISVVWAQEGAPAQLPCSPTIPLQDLSLLRRAGVTWQHQPDSGPPAPAPGHPPVPGHRPAAPYSWGPRPRRYTVLSVGPGGLRSGRLPLQPRVQLDERGRQRGDFSLWLRPARRADAGEYRATVHLRDRALSCRLRLRVGQASMTASPPGSLRTSDWVILNCSFSRPDRPASVHWFRSRGQGRVPVQGSPHHHLAESFLFLPHVGPMDSGLWGCILTYRDGFNVSIMYNLTVLGLEPATPLTVYAGAGSRVELPCRLPPAVGTQSFLTAKWAPPGGGPDLLVAGDNGDFTLRLEDVSQAQAGTYICHIRLQGQQLNATVTLAIITVTPKSFGSPGSLGKLLCEVTPASGQEHFVWSPLNTPSQRSFSGPWLEAQEAQLLSQPWQCQLHQGERLLGAAVYFTELSSPGAQRSGRAPGALRAGHLPLFLILGVLFLLLLVTGAFGFHLWRRQWRPRRFSALEQGIHPPQAQSKIEELEQEPELEPEPELERELGPEPEPGPEPEPEQL

SEQ ID NO: 577 - protein, 449 aa, synthetic construct
  REGION 1..449: note = synthetic
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 578 - protein, 214 aa, synthetic construct
  REGION 1..214: note = synthetic
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 579 - protein, 450 aa, synthetic construct
  REGION 1..450: note = synthetic
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 580 - protein, 445 aa, synthetic construct
  REGION 1..445: note = synthetic
QVQLQQWGAGLLKPSETLSLTCAVYGGSFSGYYWNWIRQPPGKGLEWVGEISHRGSTNYNPSLKSRVTISLDTSKNQFSLKLTSVTAADTAVYYCSRDEELEFRFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 581 - protein, 214 aa, synthetic construct
  REGION 1..214: note = synthetic
EIVLTQSPATLSLSPGERATLSCRASQSVSSYLAWYQQKPGQAPRLLVYGASNRATGIPARFSGSGSGTDFTLTISSLEPEDFAFYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 582 - protein, 525 aa, synthetic construct
  REGION 1..525: note = synthetic
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 583 - protein, 15 aa, synthetic construct
  REGION 1..15: note = synthetic
ENPVVHFFKNIVTPR

SEQ ID NO: 584 - DNA, 17 bp, synthetic construct
  misc_feature 1..17: note = synthetic
  source 1..17: mol_type = other DNA
agcagctctgccctcat

SEQ ID NO: 585 - DNA, 22 bp, synthetic construct
  misc_feature 1..22: note = synthetic
  source 1..22: mol_type = other DNA
gctctggctggtcttcagtatg

SEQ ID NO: 586 - DNA, 22 bp, synthetic construct
  misc_feature 1..22: note = synthetic
  source 1..22: mol_type = other DNA
ttgccgtatggttggtttgaac

SEQ ID NO: 587 - protein, 665 aa, synthetic construct
  REGION 1..665: note = hLAG3.Fc
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 588 - protein, 422 aa, synthetic construct
  REGION 1..422: note = hLAG3 extracellular domain P18627
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 589 - protein, 44 aa, synthetic construct
  REGION 1..44: note = hLAG3 epitope
LRRAGVTWQHQPDSGPPAAAPGHPLAPGPHPAAPSSWGPRPRRY